SEQUENCE LISTING (214 sequences)

SEQ ID NO: 1 - DNA, 53 bp, synthetic construct
  misc_feature 1..53: note = Description of Artificial Sequence: Synthetic primer
  source 1..53: mol_type = other DNA
tcgtcggcagcgtcagatgtgtataagagacagvbhdvccagtctggccaggg

SEQ ID NO: 2 - DNA, 52 bp, synthetic construct
  misc_feature 1..52: note = Description of Artificial Sequence: Synthetic primer
  source 1..52: mol_type = other DNA
gtctcgtgggctcggagatgtgtataagagacaggtgatgccgtagtactgg

SEQ ID NO: 3 - protein, 8 aa, synthetic construct
  REGION 1..8: note = Description of Artificial Sequence: Synthetic peptide
  VARIANT 3..4: note = X can be any Animo Acid
  VARIANT 7: note = X can be any Animo Acid
  VARIANT 8: note = X can be E or D
QPXXPFXX

SEQ ID NO: 4 - skipped

SEQ ID NO: 5 - protein, 9 aa, synthetic construct
  REGION 1..9: note = Description of Artificial Sequence: Synthetic peptide
  VARIANT 2..3: note = X can be any Animo Acid
  VARIANT 6: note = X can be D or N
  VARIANT 7..8: note = X can be any Amino Acid
RXXQEXXXF

SEQ ID NO: 6 - protein, 8 aa, synthetic construct
  REGION 1..8: note = Description of Artificial Sequence: Synthetic peptide
  VARIANT 1: note = X can be M,L,I or V
  VARIANT 3..4: note = X can be any Amino Acid
  VARIANT 7: note = X can be any Amino Acid
XEXXLFXR

SEQ ID NO: 7 - protein, 7 aa, synthetic construct
  REGION 1..7: note = Description of Artificial Sequence: Synthetic peptide
  VARIANT 2: note = X can be M or L
  VARIANT 4: note = X can be any Amino Acid
  VARIANT 6: note = X can be any Amino Acid
DXDXFXK

SEQ ID NO: 8 - protein, 7 aa, synthetic construct
  REGION 1..7: note = Description of Artificial Sequence: Synthetic peptide
  VARIANT 2..3: note = X can be any Amino Acid
  VARIANT 6: note = X can be E,N or A
FXXVCXK

SEQ ID NO: 9 - protein, 5 aa, synthetic construct
  REGION 1..5: note = Description of Artificial Sequence: Synthetic peptide
HGWTK

SEQ ID NO: 10-13 - skipped (4 entries)

SEQ ID NO: 14 - protein, 8 aa, synthetic construct
  REGION 1..8: note = Description of Artificial Sequence: Synthetic peptide
  VARIANT 3: note = X can be any Amino Acid
  VARIANT 5: note = X can be any Amino Acid
  VARIANT 7: note = X can be any Amino Acid
EGXLXMXK

SEQ ID NO: 15 - protein, 9 aa, synthetic construct
  REGION 1..9: note = Description of Artificial Sequence: Synthetic peptide
  VARIANT 2..3: note = X can be any Amino Acid
  VARIANT 6..7: note = X can be any Amino Acid
  VARIANT 8: note = X can be W or Y
FXXELXXXF

SEQ ID NO: 16-17 - skipped (2 entries)

SEQ ID NO: 18 - protein, 7 aa, synthetic construct
  REGION 1..7: note = Description of Artificial Sequence: Synthetic peptide
  VARIANT 2: note = X can be any Amino Acid
  VARIANT 4: note = X can be M or Q
  VARIANT 6: note = X can be any Amino Acid
QXPXLXK

SEQ ID NO: 19 - protein, 10 aa, synthetic construct
  REGION 1..10: note = Description of Artificial Sequence: Synthetic peptide
  VARIANT 2..3: note = X can be any Amino Acid
  VARIANT 5: note = X can be L or M
  VARIANT 6: note = X can be any Amino Acid
  VARIANT 8..9: note = X can be any Amino Acid
QXXCXXKXXF

SEQ ID NO: 20 - protein, 9 aa, synthetic construct
  REGION 1..9: note = Description of Artificial Sequence: Synthetic peptide
  VARIANT 2..4: note = X can be any Amino Acid
  VARIANT 8: note = X can be any Amino Acid
CXXXPCMXK

SEQ ID NO: 21 - protein, 7 aa, synthetic construct
  REGION 1..7: note = Description of Artificial Sequence: Synthetic peptide
  VARIANT 2: note = X can be any Amino Acid
  VARIANT 5: note = X can be any Amino Acid
  VARIANT 6: note = X can be S or T
KXQTXXM

SEQ ID NO: 22 - protein, 7 aa, synthetic construct
  REGION 1..7: note = Description of Artificial Sequence: Synthetic peptide
  VARIANT 2: note = X can be any Amino Acid
  VARIANT 3: note = X can be A or S
  VARIANT 5: note = X can be any Amino Acid
QXXMXPK

SEQ ID NO: 23 - protein, 9 aa, synthetic construct
  REGION 1..9: note = Description of Artificial Sequence: Synthetic peptide
  VARIANT 1: note = X can be T or S
  VARIANT 4..5: note = X can be any Amino Acid
  VARIANT 7..8: note = X can be any Amino Acid
XPMXXAXXK

SEQ ID NO: 24 - protein, 8 aa, synthetic construct
  REGION 1..8: note = Description of Artificial Sequence: Synthetic peptide
  VARIANT 3..4: note = X can be any Amino Acid
  VARIANT 6: note = X can be T or S
  VARIANT 7: note = X can be any Amino Acid
STXXLXXK

SEQ ID NO: 25-26 - skipped (2 entries)

SEQ ID NO: 27 - protein, 6 aa, synthetic construct
  REGION 1..6: note = Description of Artificial Sequence: Synthetic peptide
  VARIANT 2: note = X can be E, D or T
  VARIANT 5: note = X can be any Amino Acid
PXWRXY

SEQ ID NO: 28 - protein, 6 aa, synthetic construct
  REGION 1..6: note = Description of Artificial Sequence: Synthetic peptide
  VARIANT 2: note = X can be any Amino Acid
  VARIANT 5: note = X can be V or I
PXWRXY

SEQ ID NO: 29 - protein, 9 aa, synthetic construct
  REGION 1..9: note = Description of Artificial Sequence: Synthetic peptide
  VARIANT 2: note = X can be any Amino Acid
  VARIANT 6..8: note = X can be any Amino Acid
  VARIANT 9: note = X can be D, G, E, S or N
DXSGIXXXX

SEQ ID NO: 30 - protein, 9 aa, synthetic construct
  REGION 1..9: note = Description of Artificial Sequence: Synthetic peptide
  VARIANT 1: note = X can be A, P or C
  VARIANT 2..4: note = X can be any Amino Acid
  VARIANT 6: note = X can be any Amino Acid
XXXXDXSGI

SEQ ID NO: 31 - protein, 5 aa, synthetic construct
  REGION 1..5: note = Description of Artificial Sequence: Synthetic peptide
  VARIANT 5: note = X can be L, M or I
KTPPX

SEQ ID NO: 32 - protein, 5 aa, synthetic construct
  REGION 1..5: note = Description of Artificial Sequence: Synthetic peptide
LPFQQ

SEQ ID NO: 33 - skipped

SEQ ID NO: 34 - protein, 7 aa, synthetic construct
  REGION 1..7: note = Description of Artificial Sequence: Synthetic peptide
  VARIANT 3..4: note = X can be any Amino Acid
KWXXFQQ

SEQ ID NO: 35 - skipped

SEQ ID NO: 36 - protein, 8 aa, synthetic construct
  REGION 1..8: note = Description of Artificial Sequence: Synthetic peptide
  VARIANT 2..3: note = X can be any Amino Acid
  VARIANT 7: note = X can be any Amino Acid
CXXFIAXC

SEQ ID NO: 37 - protein, 10 aa, synthetic construct
  REGION 1..10: note = Description of Artificial Sequence: Synthetic peptide
  VARIANT 2..3: note = X can be any Amino Acid
  VARIANT 6: note = X can be any Amino Acid
  VARIANT 8..9: note = X can be any Amino Acid
  VARIANT 7: note = X can be C or A
CXXYWXXXXK

SEQ ID NO: 38 - protein, 8 aa, synthetic construct
  REGION 1..8: note = Description of Artificial Sequence: Synthetic peptide
  VARIANT 1: note = X can be L, M or I
  VARIANT 4..6: note = X can be any Amino Acid
XLPXXXLD

SEQ ID NO: 39 - protein, 9 aa, synthetic construct
  REGION 1..9: note = Description of Artificial Sequence: Synthetic peptide
  VARIANT 2..3: note = X can be any Amino Acid
  VARIANT 6..7: note = X can be any Amino Acid
KXXGPXXLY

SEQ ID NO: 40 - protein, 5 aa, synthetic construct
  REGION 1..5: note = Description of Artificial Sequence: Synthetic peptide
  VARIANT 5: note = X can be L or I
QGPTX

SEQ ID NO: 41 - protein, 10 aa, synthetic construct
  REGION 1..10: note = Description of Artificial Sequence: Synthetic peptide
  VARIANT 2: note = X can be any Amino Acid
  VARIANT 4: note = X can be T, C or A
  VARIANT 6..9: note = X can be any Amino Acid
KXGXPXXXXP

SEQ ID NO: 42-44 - skipped (3 entries)

SEQ ID NO: 45 - protein, 8 aa, synthetic construct
  REGION 1..8: note = Description of Artificial Sequence: Synthetic peptide
  VARIANT 2..3: note = X can be any Amino Acid
  VARIANT 5: note = X can be K or R
  VARIANT 7: note = X can be any Amino Acid
CXXCXAXS

SEQ ID NO: 46-49 - skipped (4 entries)

SEQ ID NO: 50 - protein, 6 aa, synthetic construct
  REGION 1..6: note = Description of Artificial Sequence: Synthetic peptide
  VARIANT 2: note = X can be any Amino Acid
  VARIANT 3: note = X can be R or K
WXXMAD

SEQ ID NO: 51 - protein, 7 aa, synthetic construct
  REGION 1..7: note = Description of Artificial Sequence: Synthetic peptide
  VARIANT 5..6: note = X can be any Amino Acid
RMADXXR

SEQ ID NO: 52 - protein, 8 aa, synthetic construct
  REGION 1..8: note = Description of Artificial Sequence: Synthetic peptide
  VARIANT 3..5: note = X can be any Amino Acid
  VARIANT 8: note = X can be T, S or R
PQXXXDKX

SEQ ID NO: 53-54 - skipped (2 entries)

SEQ ID NO: 55 - protein, 5 aa, synthetic construct
  REGION 1..5: note = Description of Artificial Sequence: Synthetic peptide
  VARIANT 2: note = X can be S, K or H
DXNGT

SEQ ID NO: 56-58 - skipped (3 entries)

SEQ ID NO: 59 - protein, 9 aa, synthetic construct
  REGION 1..9: note = Description of Artificial Sequence: Synthetic peptide
  VARIANT 2..3: note = X can be any Amino Acid
  VARIANT 5..6: note = X can be any Amino Acid
  VARIANT 8: note = X can be S or T
CXXDXXGXC

SEQ ID NO: 60 - skipped

SEQ ID NO: 61 - protein, 7 aa, synthetic construct
  REGION 1..7: note = Description of Artificial Sequence: Synthetic peptide
  VARIANT 4: note = X can be T or S
  VARIANT 5..6: note = X can be any Amino Acid
PNGXXXP

SEQ ID NO: 62 - protein, 6 aa, synthetic construct
  REGION 1..6: note = Description of Artificial Sequence: Synthetic peptide
  VARIANT 2: note = X can be any Amino Acid
GXVDWD

SEQ ID NO: 63 - protein, 10 aa, synthetic construct
  REGION 1..10: note = Description of Artificial Sequence: Synthetic peptide
  VARIANT 2: note = X can be any Amino Acid
  VARIANT 4: note = X can be any Amino Acid
  VARIANT 6: note = X can be any Amino Acid
  VARIANT 8..9: note = X can be any Amino Acid
CXPXCXEXXW

SEQ ID NO: 64 - skipped

SEQ ID NO: 65 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 66 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 67 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 68 - protein, 97 aa, Severe acute respiratory syndrome coronavirus 2
MDPKISEMHPALRLVDPQIQLAVTRMENAVGRDQNNVGPKVYPIILRLGSPLSLNMARKTLNSLEDKAFQLTPIAVQMTKLATTEELPDEFVVVTVK

SEQ ID NO: 69 - protein, 7095 aa, Severe acute respiratory syndrome coronavirus 2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVN

SEQ ID NO: 70 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 71-73 - skipped (3 entries)

SEQ ID NO: 74 - protein, 7 aa, synthetic construct
  REGION 1..7: note = Description of Artificial Sequence: Synthetic peptide
  VARIANT 3: note = X can be any Amino Acid
  VARIANT 4: note = X can be A or P
  VARIANT 6: note = X can be any Amino Acid
KWXXFXQ

SEQ ID NO: 75 - skipped

SEQ ID NO: 76 - protein, 8 aa, synthetic construct
  REGION 1..8: note = Description of Artificial Sequence: Synthetic peptide
  VARIANT 3..4: note = X can be any Amino Acid
  VARIANT 5: note = X can be A or P
  VARIANT 7: note = X can be any Amino Acid
WKXXXFXQ

SEQ ID NO: 77 - skipped

SEQ ID NO: 78 - protein, 7 aa, synthetic construct
  REGION 1..7: note = Description of Artificial Sequence: Synthetic peptide
  VARIANT 2: note = X can be any Amino Acid
  VARIANT 4: note = X can be any Amino Acid
  VARIANT 5: note = X can be S or T
RXPXXLE

SEQ ID NO: 79 - protein, 6 aa, synthetic construct
  REGION 1..6: note = Description of Artificial Sequence: Synthetic peptide
  VARIANT 2: note = X can be any Amino Acid
  VARIANT 5: note = X can be I or V
PXWRXY

SEQ ID NO: 80 - protein, 6 aa, synthetic construct
  REGION 1..6: note = Description of Artificial Sequence: Synthetic peptide
  VARIANT 2: note = X can be D or E
  VARIANT 5: note = X can be any Amino Acid
PXWRXY

SEQ ID NO: 81 - skipped

SEQ ID NO: 82 - protein, 5 aa, synthetic construct
  REGION 1..5: note = Description of Artificial Sequence: Synthetic peptide
  VARIANT 3: note = X can be I, L, M or V
RSXAS

SEQ ID NO: 83 - protein, 5 aa, synthetic construct
  REGION 1..5: note = Description of Artificial Sequence: Synthetic peptide
RSVAS

SEQ ID NO: 84 - skipped

SEQ ID NO: 85 - protein, 6 aa, synthetic construct
  REGION 1..6: note = Description of Artificial Sequence: Synthetic peptide
  VARIANT 3: note = X can be any Amino Acid
  VARIANT 5: note = X can be L or M
ETXSXG

SEQ ID NO: 86 - protein, 5 aa, synthetic construct
  REGION 1..5: note = Description of Artificial Sequence: Synthetic peptide
  VARIANT 5: note = X can be I, L or M
KTPPX

SEQ ID NO: 87 - protein, 9 aa, synthetic construct
  REGION 1..9: note = Description of Artificial Sequence: Synthetic peptide
  VARIANT 2: note = X can be E, H or S
  VARIANT 3..4: note = X can be any Amino Acid
  VARIANT 6..7: note = X can be any Amino Acid
RXXXEXXLF

SEQ ID NO: 88 - protein, 9 aa, synthetic construct
  REGION 1..9: note = Description of Artificial Sequence: Synthetic peptide
  VARIANT 2..3: note = X can be any Amino Acid
  VARIANT 6..7: note = X can be any Amino Acid
RXXQEXXLF

SEQ ID NO: 89 - skipped

SEQ ID NO: 90 - protein, 8 aa, synthetic construct
  REGION 1..8: note = Description of Artificial Sequence: Synthetic peptide
  VARIANT 2..3: note = X can be any Amino Acid
  VARIANT 6: note = X can be any Amino Acid
SXXEDXLF

SEQ ID NO: 91 - protein, 8 aa, synthetic construct
  REGION 1..8: note = Description of Artificial Sequence: Synthetic peptide
  VARIANT 2: note = X can be any Amino Acid
  VARIANT 4: note = X can be any Amino Acid
  VARIANT 7: note = X can be any Amino Acid
  VARIANT 8: note = X can be I, L or M
KXSXIEXX

SEQ ID NO: 92 - protein, 8 aa, synthetic construct
  REGION 1..8: note = Description of Artificial Sequence: Synthetic peptide
  VARIANT 1: note = X can be I, L, M or V
  VARIANT 3..4: note = X can be any Amino Acid
  VARIANT 7: note = X can be any Amino Acid
XEXXLFXR

SEQ ID NO: 93 - protein, 8 aa, synthetic construct
  REGION 1..8: note = Description of Artificial Sequence: Synthetic peptide
  VARIANT 3: note = X can be any Amino Acid
  VARIANT 5..6: note = X can be any Amino Acid
KEXLXXWF

SEQ ID NO: 94 - protein, 9 aa, synthetic construct
  REGION 1..9: note = Description of Artificial Sequence: Synthetic peptide
  VARIANT 4: note = X can be any Amino Acid
  VARIANT 6..8: note = X can be any Amino Acid
FEQXLXXXF

SEQ ID NO: 95 - protein, 5 aa, synthetic construct
  REGION 1..5: note = Description of Artificial Sequence: Synthetic peptide
  VARIANT 2: note = X can be L, M or Y
DXSGI

SEQ ID NO: 96 - protein, 9 aa, synthetic construct
  REGION 1..9: note = Description of Artificial Sequence: Synthetic peptide
  VARIANT 1: note = X can be A, C or P
  VARIANT 2..4: note = X can be any Amino Acid
  VARIANT 6: note = X can be any Amino Acid
XXXXDXSGI

SEQ ID NO: 97 - skipped

SEQ ID NO: 98 - protein, 6 aa, synthetic construct
  REGION 1..6: note = Description of Artificial Sequence: Synthetic peptide
  VARIANT 5: note = X can be any Amino Acid
QGPTXP

SEQ ID NO: 99 - protein, 9 aa, synthetic construct
  REGION 1..9: note = Description of Artificial Sequence: Synthetic peptide
  VARIANT 2: note = X can be any Amino Acid
  VARIANT 6..8: note = X can be any Amino Acid
HXQCPXXXK

SEQ ID NO: 100 - skipped

SEQ ID NO: 101 - protein, 8 aa, synthetic construct
  REGION 1..8: note = Description of Artificial Sequence: Synthetic peptide
  VARIANT 2: note = X can be any Amino Acid
  VARIANT 3: note = X can be I or L
  VARIANT 5..6: note = X can be any Amino Acid
LXXLXXLN

SEQ ID NO: 102-104 - skipped (3 entries)

SEQ ID NO: 105 - protein, 7 aa, synthetic construct
  REGION 1..7: note = Description of Artificial Sequence: Synthetic peptide
  VARIANT 1: note = X can be F or Y
  VARIANT 3: note = X can be any Amino Acid
  VARIANT 5: note = X can be any Amino Acid
XPXTXPK

SEQ ID NO: 106 - protein, 8 aa, synthetic construct
  REGION 1..8: note = Description of Artificial Sequence: Synthetic peptide
  VARIANT 4..6: note = X can be any Amino Acid
  VARIANT 7: note = X can be I or L
LLPXXXXD

SEQ ID NO: 107 - protein, 8 aa, synthetic construct
  REGION 1..8: note = Description of Artificial Sequence: Synthetic peptide
  VARIANT 1: note = X can be I, L or M
  VARIANT 4..6: note = X can be any Amino Acid
XLPXXXLD

SEQ ID NO: 108 - protein, 7 aa, synthetic construct
  REGION 1..7: note = Description of Artificial Sequence: Synthetic peptide
  VARIANT 2: note = X can be L or M
  VARIANT 4: note = X can be any Amino Acid
  VARIANT 6: note = X can be any Amino Acid
DXDXFXK

SEQ ID NO: 109-110 - skipped (2 entries)

SEQ ID NO: 111 - protein, 6 aa, synthetic construct
  REGION 1..6: note = Description of Artificial Sequence: Synthetic peptide
  VARIANT 2: note = X can be any Amino Acid
  VARIANT 5: note = X can be any Amino Acid
DXAGXK

SEQ ID NO: 112 - protein, 8 aa, synthetic construct
  REGION 1..8: note = Description of Artificial Sequence: Synthetic peptide
  VARIANT 3: note = X can be any Amino Acid
  VARIANT 4: note = X can be A or G
  VARIANT 5: note = X can be any Amino Acid
  VARIANT 7: note = X can be any Amino Acid
DCXXXKXC

SEQ ID NO: 113-114 - skipped (2 entries)

SEQ ID NO: 115 - protein, 7 aa, synthetic construct
  REGION 1..7: note = Description of Artificial Sequence: Synthetic peptide
  VARIANT 1: note = X can be G, S or T
  VARIANT 3: note = X can be any Amino Acid
  VARIANT 6: note = X can be any Amino Acid
XPXQYXD

SEQ ID NO: 116 - protein, 10 aa, synthetic construct
  REGION 1..10: note = Description of Artificial Sequence: Synthetic peptide
  VARIANT 2..5: note = X can be any Amino Acid
  VARIANT 9: note = X can be any Amino Acid
CXXXXCVMXK

SEQ ID NO: 117 - protein, 7 aa, synthetic construct
  REGION 1..7: note = Description of Artificial Sequence: Synthetic peptide
  VARIANT 2..3: note = X can be any Amino Acid
  VARIANT 5: note = X can be M or T
FXXVXRK

SEQ ID NO: 118 - protein, 7 aa, synthetic construct
  REGION 1..7: note = Description of Artificial Sequence: Synthetic peptide
  VARIANT 2: note = X can be any Amino Acid
  VARIANT 6: note = X can be any Amino Acid
FXQVCXK

SEQ ID NO: 119 - skipped

SEQ ID NO: 120 - protein, 8 aa, synthetic construct
  REGION 1..8: note = Description of Artificial Sequence: Synthetic peptide
  VARIANT 3..4: note = X can be any Amino Acid
  VARIANT 6: note = X can be L, M or T
  VARIANT 7: note = X can be any Amino Acid
WWXXLXXK

SEQ ID NO: 121-126 - skipped (6 entries)

SEQ ID NO: 127 - protein, 5 aa, synthetic construct
  REGION 1..5: note = Description of Artificial Sequence: Synthetic peptide
  VARIANT 3: note = X can be A or G
MWXGK

SEQ ID NO: 128 - protein, 5 aa, synthetic construct
  REGION 1..5: note = Description of Artificial Sequence: Synthetic peptide
  VARIANT 4: note = X can be A or T
MTAXK

SEQ ID NO: 129 - protein, 9 aa, synthetic construct
  REGION 1..9: note = Description of Artificial Sequence: Synthetic peptide
  VARIANT 4..6: note = X can be any Amino Acid
  VARIANT 8: note = X can be any Amino Acid
  VARIANT 7: note = X can be S or T
TPMXXXXXK

SEQ ID NO: 130 - protein, 8 aa, synthetic construct
  REGION 1..8: note = Description of Artificial Sequence: Synthetic peptide
  VARIANT 3..4: note = X can be any Amino Acid
  VARIANT 7: note = X can be any Amino Acid
  VARIANT 6: note = X can be S or T
STXXLXXK

SEQ ID NO: 131 - skipped

SEQ ID NO: 132 - protein, 9 aa, synthetic construct
  REGION 1..9: note = Description of Artificial Sequence: Synthetic peptide
  VARIANT 1: note = X can be S or T
  VARIANT 4..5: note = X can be any Amino Acid
  VARIANT 7..8: note = X can be any Amino Acid
XPMXXAXXK

SEQ ID NO: 133-134 - skipped (2 entries)

SEQ ID NO: 135 - protein, 7 aa, synthetic construct
  REGION 1..7: note = Description of Artificial Sequence: Synthetic peptide
  VARIANT 3: note = X can be any Amino Acid
  VARIANT 6: note = X can be any Amino Acid
  VARIANT 5: note = X can be A, M or S
SAXYXXK

SEQ ID NO: 136 - protein, 8 aa, synthetic construct
  REGION 1..8: note = Description of Artificial Sequence: Synthetic peptide
  VARIANT 2: note = X can be any Amino Acid
  VARIANT 6..7: note = X can be any Amino Acid
  VARIANT 8: note = X can be F, M or W
QXSFKXXX

SEQ ID NO: 137 - skipped

SEQ ID NO: 138 - protein, 8 aa, synthetic construct
  REGION 1..8: note = Description of Artificial Sequence: Synthetic peptide
  VARIANT 2: note = X can be any Amino Acid
  VARIANT 6..7: note = X can be any Amino Acid
  VARIANT 8: note = X can be F or W
QXSFKXXX

SEQ ID NO: 139 - protein, 8 aa, synthetic construct
  REGION 1..8: note = Description of Artificial Sequence: Synthetic peptide
  VARIANT 2..3: note = X can be any Amino Acid
  VARIANT 7: note = X can be any Amino Acid
KXXIPYXD

SEQ ID NO: 140 - skipped

SEQ ID NO: 141 - protein, 7 aa, synthetic construct
  REGION 1..7: note = Description of Artificial Sequence: Synthetic peptide
  VARIANT 3: note = X can be any Amino Acid
  VARIANT 6: note = X can be any Amino Acid
KMXPWXQ

SEQ ID NO: 142 - protein, 8 aa, synthetic construct
  REGION 1..8: note = Description of Artificial Sequence: Synthetic peptide
  VARIANT 2: note = X can be any Amino Acid
  VARIANT 4: note = X can be any Amino Acid
  VARIANT 7: note = X can be any Amino Acid
CXLXWSXC

SEQ ID NO: 143 - skipped

SEQ ID NO: 144 - protein, 6 aa, synthetic construct
  REGION 1..6: note = Description of Artificial Sequence: Synthetic peptide
  VARIANT 4: note = X can be any Amino Acid
  VARIANT 3: note = X can be D or E
WFXXID

SEQ ID NO: 145 - protein, 5 aa, synthetic construct
  REGION 1..5: note = Description of Artificial Sequence: Synthetic peptide
  VARIANT 2: note = X can be F or H
RXPEK

SEQ ID NO: 146 - skipped

SEQ ID NO: 147 - protein, 7 aa, synthetic construct
  REGION 1..7: note = Description of Artificial Sequence: Synthetic peptide
  VARIANT 2: note = X can be any Amino Acid
  VARIANT 4: note = X can be any Amino Acid
  VARIANT 5: note = X can be I, L or M
CXEXXLF

SEQ ID NO: 148 - protein, 8 aa, synthetic construct
  REGION 1..8: note = Description of Artificial Sequence: Synthetic peptide
  VARIANT 2: note = X can be any Amino Acid
  VARIANT 4..5: note = X can be any Amino Acid
  VARIANT 7: note = X can be L or M
PXWXXGXD

SEQ ID NO: 149 - protein, 7 aa, synthetic construct
  REGION 1..7: note = Description of Artificial Sequence: Synthetic peptide
  VARIANT 2..3: note = X can be any Amino Acid
  VARIANT 4: note = X can be M or S
AXXXRKP

SEQ ID NO: 150 - protein, 10 aa, synthetic construct
  REGION 1..10: note = Description of Artificial Sequence: Synthetic peptide
  VARIANT 2: note = X can be any Amino Acid
  VARIANT 6..9: note = X can be any Amino Acid
  VARIANT 4: note = X can be A, C or T
KXGXPXXXXP

SEQ ID NO: 151 - protein, 7 aa, synthetic construct
  REGION 1..7: note = Description of Artificial Sequence: Synthetic peptide
  VARIANT 3: note = X can be any Amino Acid
  VARIANT 6: note = X can be any Amino Acid
YWXYFXK

SEQ ID NO: 152 - protein, 10 aa, synthetic construct
  REGION 1..10: note = Description of Artificial Sequence: Synthetic peptide
  VARIANT 2..3: note = X can be any Amino Acid
  VARIANT 6: note = X can be any Amino Acid
  VARIANT 8..9: note = X can be any Amino Acid
  VARIANT 7: note = X can be A or C
CXXYWXXXXK

SEQ ID NO: 153 - protein, 9 aa, synthetic construct
  REGION 1..9: note = Description of Artificial Sequence: Synthetic peptide
  VARIANT 2: note = X can be any Amino Acid
  VARIANT 4: note = X can be any Amino Acid
  VARIANT 6: note = X can be any Amino Acid
  VARIANT 8: note = X can be any Amino Acid
CXVXPXMXK

SEQ ID NO: 154 - protein, 8 aa, synthetic construct
  REGION 1..8: note = Description of Artificial Sequence: Synthetic peptide
  VARIANT 3: note = X can be any Amino Acid
  VARIANT 5: note = X can be any Amino Acid
  VARIANT 7: note = X can be any Amino Acid
YCXAXKXC

SEQ ID NO: 155 - protein, 6 aa, synthetic construct
  REGION 1..6: note = Description of Artificial Sequence: Synthetic peptide
  VARIANT 2: note = X can be any Amino Acid
  VARIANT 4: note = X can be F or W
KXPXCP

SEQ ID NO: 156 - skipped

SEQ ID NO: 157 - protein, 6 aa, synthetic construct
  REGION 1..6: note = Description of Artificial Sequence: Synthetic peptide
  VARIANT 5: note = X can be any Amino Acid
  VARIANT 6: note = X can be A, H or R
RMADXX

SEQ ID NO: 158 - protein, 6 aa, synthetic construct
  REGION 1..6: note = Description of Artificial Sequence: Synthetic peptide
  VARIANT 3: note = X can be any Amino Acid
WAXMAD

SEQ ID NO: 159-161 - skipped (3 entries)

SEQ ID NO: 162 - protein, 10 aa, synthetic construct
  REGION 1..10: note = Description of Artificial Sequence: Synthetic peptide
  VARIANT 2..3: note = X can be any Amino Acid
  VARIANT 6: note = X can be any Amino Acid
  VARIANT 8..9: note = X can be any Amino Acid
  VARIANT 10: note = X can be H, P or W
PXXRAXSXXX

SEQ ID NO: 163 - skipped

SEQ ID NO: 164 - protein, 10 aa, synthetic construct
  REGION 1..10: note = Description of Artificial Sequence: Synthetic peptide
  VARIANT 2..4: note = X can be any Amino Acid
  VARIANT 7: note = X can be any Amino Acid
  VARIANT 9: note = X can be any Amino Acid
PXXXRAXSXG

SEQ ID NO: 165-168 - skipped (4 entries)

SEQ ID NO: 169 - protein, 10 aa, synthetic construct
  REGION 1..10: note = Description of Artificial Sequence: Synthetic peptide
  VARIANT 3: note = X can be any Amino Acid
  VARIANT 5..9: note = X can be any Amino Acid
KAXSXXXXXQ

SEQ ID NO: 170-171 - skipped (2 entries)

SEQ ID NO: 172 - protein, 8 aa, synthetic construct
  REGION 1..8: note = Description of Artificial Sequence: Synthetic peptide
  VARIANT 2..3: note = X can be any Amino Acid
  VARIANT 7: note = X can be any Amino Acid
  VARIANT 8: note = X can be A or S
IXXAVSXX

SEQ ID NO: 173 - protein, 8 aa, synthetic construct
  REGION 1..8: note = Description of Artificial Sequence: Synthetic peptide
  VARIANT 3: note = X can be any Amino Acid
  VARIANT 5..7: note = X can be any Amino Acid
KAXSXXXY

SEQ ID NO: 174 - skipped

SEQ ID NO: 175 - protein, 8 aa, synthetic construct
  REGION 1..8: note = Description of Artificial Sequence: Synthetic peptide
  VARIANT 3: note = X can be any Amino Acid
  VARIANT 6..7: note = X can be any Amino Acid
  VARIANT 1: note = X can be K or R
XAXSMXXR

SEQ ID NO: 176-177 - skipped (2 entries)

SEQ ID NO: 178 - protein, 9 aa, synthetic construct
  REGION 1..9: note = Description of Artificial Sequence: Synthetic peptide
  VARIANT 1: note = X can be F or Y
  VARIANT 2: note = X can be any Amino Acid
  VARIANT 5: note = X can be any Amino Acid
  VARIANT 7..8: note = X can be any Amino Acid
XXKAXSXXY

SEQ ID NO: 179 - skipped

SEQ ID NO: 180 - protein, 6 aa, synthetic construct
  REGION 1..6: note = Description of Artificial Sequence: Synthetic peptide
  VARIANT 2: note = X can be any Amino Acid
  VARIANT 1: note = X can be F or Y
XXRAMS

SEQ ID NO: 181 - skipped

SEQ ID NO: 182 - protein, 7 aa, synthetic construct
  REGION 1..7: note = Description of Artificial Sequence: Synthetic peptide
  VARIANT 1: note = X can be F or W
  VARIANT 2: note = X can be any Amino Acid
  VARIANT 6: note = X can be any Amino Acid
XXRRDXG

SEQ ID NO: 183 - skipped

SEQ ID NO: 184 - protein, 9 aa, synthetic construct
  REGION 1..9: note = Description of Artificial Sequence: Synthetic peptide
  VARIANT 2: note = X can be any Amino Acid
  VARIANT 6..8: note = X can be any Amino Acid
  VARIANT 4: note = X can be A or K
FXRXDXXXP

SEQ ID NO: 185-187 - skipped (3 entries)

SEQ ID NO: 188 - protein, 5 aa, synthetic construct
  REGION 1..5: note = Description of Artificial Sequence: Synthetic peptide
  VARIANT 5: note = X can be S or T
DSNGX

SEQ ID NO: 189 - protein, 6 aa, synthetic construct
  REGION 1..6: note = Description of Artificial Sequence: Synthetic peptide
  VARIANT 4: note = X can be any Amino Acid
  VARIANT 1: note = X can be R or Y
XADXNG

SEQ ID NO: 190-194 - skipped (5 entries)

SEQ ID NO: 195 - protein, 5 aa, synthetic construct
  REGION 1..5: note = Description of Artificial Sequence: Synthetic peptide
  VARIANT 5: note = X can be F or Y
IPGSX

SEQ ID NO: 196 - protein, 6 aa, synthetic construct
  REGION 1..6: note = Description of Artificial Sequence: Synthetic peptide
  VARIANT 5: note = X can be any Amino Acid
CSTIXC

SEQ ID NO: 197 - skipped

SEQ ID NO: 198 - protein, 8 aa, synthetic construct
  REGION 1..8: note = Description of Artificial Sequence: Synthetic peptide
  VARIANT 3: note = X can be S or T
  VARIANT 4..5: note = X can be any Amino Acid
  VARIANT 7: note = X can be any Amino Acid
RDXXXPXR

SEQ ID NO: 199 - skipped

SEQ ID NO: 200 - protein, 7 aa, synthetic construct
  REGION 1..7: note = Description of Artificial Sequence: Synthetic peptide
  VARIANT 1: note = X can be F or I
  VARIANT 3: note = X can be any Amino Acid
  VARIANT 5: note = X can be any Amino Acid
XAXEXDK

SEQ ID NO: 201 - protein, 6 aa, synthetic construct
  REGION 1..6: note = Description of Artificial Sequence: Synthetic peptide
  VARIANT 1: note = X can be D or N
  VARIANT 4: note = X can be any Amino Acid
XAAXAL

SEQ ID NO: 202-203 - skipped (2 entries)

SEQ ID NO: 204 - protein, 10 aa, Severe acute respiratory syndrome coronavirus 2
MFVFLVLLPL

SEQ ID NO: 205 - protein, 5 aa, Severe acute respiratory syndrome coronavirus 2
MFVFL

SEQ ID NO: 206 - protein, 5 aa, Severe acute respiratory syndrome coronavirus 2
FVFLV

SEQ ID NO: 207 - protein, 5 aa, Severe acute respiratory syndrome coronavirus 2
VFLVL

SEQ ID NO: 208 - protein, 5 aa, Severe acute respiratory syndrome coronavirus 2
FLVLL

SEQ ID NO: 209 - protein, 5 aa, Severe acute respiratory syndrome coronavirus 2
LVLLP

SEQ ID NO: 210 - protein, 6 aa, Severe acute respiratory syndrome coronavirus 2
MFVFLV

SEQ ID NO: 211 - protein, 6 aa, Severe acute respiratory syndrome coronavirus 2
FVFLVL

SEQ ID NO: 212 - protein, 6 aa, Severe acute respiratory syndrome coronavirus 2
VFLVLL

SEQ ID NO: 213 - protein, 6 aa, Severe acute respiratory syndrome coronavirus 2
FLVLLP

SEQ ID NO: 214 - protein, 6 aa, Severe acute respiratory syndrome coronavirus 2
LVLLPL

(19) United States
(12) Patent Application Publication
(10) Pub. No.: US 2023/0288421 A1
(43) Pub. Date: Sep. 14, 2023

(54) SARS-COV-2 SERUM ANTIBODY PROFILING

(71) Applicant: Serimmune Inc., Goleta, CA (US)

(72) Inventors: Winston A. Haynes, Santa Barbara, CA (US); Patrick Sean Daugherty, Goleta, CA (US); Kathryn Vinaya Louise Kamath, Santa Barbara, CA (US); Jack Ryan Reifert, Santa Barbara, CA (US); Abhilash Dhal, (US); John Chul-yong Shon, Portola Valley, CA (US)

(21) Appl. No.: 18/145,772

(22) Filed: Dec. 22, 2022

Related U.S. Application Data
(63) Continuation of application No. PCT/US2021/038960, filed on Jun. 24, 2021.
(60) Provisional application No. 63/114,477, filed on Nov. 16, 2020.
(60) Provisional application No. 63/043,470, filed on Jun. 24, 2020.
(60) Provisional application No. 63/043,479, filed on Jun. 24, 2020.

(51) Int. Cl. G01N 33/569; G01N 33/543; C07K 7/06

(52) U.S. Cl. CPC G01N 33/56983; G01N 33/54386; G01N 33/54326; C07K 7/06; G01N 2469/20; G01N 2333/165

(57) ABSTRACT

Disclosed herein are reagents for use in antibody profiling platforms, such as biopanning and Digital Serology, specifically for eptiope motifs directed to SARS-CoV-2. Also disclosed herein are kits, method of manufacturing, and methods of using the same.

### CROSS REFERENCE TO RELATED APPLICATIONS

This application is a continuation of International Application No. PCT/US2021/038960, filed Jun. 24, 2021, which claims priority to and the benefit of U.S. Provisional Application Nos. 63/114,477 filed Nov. 16, 2020, 63/043,479 filed Jun. 24, 2020, and 63/043,470 filed Jun. 24, 2020 each of which is hereby incorporated in its entirety by reference for all purposes.

### SEQUENCE LISTING

The instant application contains a Sequence Listing which has been submitted via Patent Center and is hereby incorporated by reference in its entirety. Said XML copy, created on Jan. 12, 2023, is named SUI-009WOC1.xml, and is 2,83,921 bytes in size.

### BACKGROUND

Serology is the practice of identifying components of serum samples. In particular, several technologies exist in the field for identifying antibodies present in serum. For example, biopanning using peptide displays is a technique used to enrich for antibodies specific to particular protein targets. Biopanning assays have also been applied to epitope identification.

Coronaviruses are one three types of viruses that cause the common cold. The four most common strains in the United States are NL63, 229E (alpha coronaviruses) and OC43, HKU1 (beta coronaviruses). In 2003, a novel coronavirus responsible for SARS (Sudden Acute Respiratory Syndrome) emerged that was far more virulent and lethal and any of the other known strains. In 2012, a second virulent strain of coronavirus, MERS (Middle Eastern Respiratory Syndrome) emerged and also exhibited a high mortality rate. In 2019 a third novel and virulent strain of coronavirus, SARS CoV-2, rapidly spread throughout the world causing the largest and most lethal pandemic since the Spanish flu. Identification of those who have been infected, developed and antibody response and are immune is a critical to vaccine and therapeutic development, as well as serosurveillance to understand immunity within the population and control viral spread. SARS CoV-2 has multiple regions of weak homology to the other coronaviruses, for example in the receptor binding domain which is thought to be a target for neutralizing antibodies. For this reason, it has been speculated that the lethality of the virus may result from lack of protective antibodies from pre-existing coronavirus infections. However, there are also multiple regions of high homology between the coronaviruses. While antibodies to these domains may not be protective, they may contain shared, immunodominant epitopes that confound the diagnostic accuracy of serological tests. SERA profiles human antibody repertoires to discover antibody epitopes, in the form of peptide motifs, specific to the array of infections to which humans are exposed.

Absent from the field are standardized SARS-CoV-2 diagnostic tests with improved sensitivity and specificity.

### SUMMARY

Serum Epitope Repertoire Analysis (SERA) utilizes bacterial display peptide library technology coupled with next-generation sequencing to profile antibody repertoires and identify IgM and IgG antigenic epitopes, in the form of peptide motifs, that are diagnostic for SARS-CoV-2 infection. The motifs identified are enriched and highly specific to SARS-CoV-2 patients.

Unlike whole antigens or protein lysates that are routinely used for detection of disease specific antibodies, SERA identifies only the regions of the protein that are the immunodominant antibody targets. For this reason, SERA is uniquely suited to discover epitopes that are shared amongst the common corona viruses versus those that are specific to SARS CoV-2. Panels of IgG and IgM epitopes and epitope motifs have been developed that together are diagnostic for CoV-2 and exhibit high specificity within a database of 1500 pre-pandemic repertoires and that are useful for the detection of asymptomatic CoV-2 exposure, kinetics of immune response and serostatus in the population over time.

Accordingly, the invention described herein provides compositions and methods directed to epitopes associated with SARS-CoV-2.

Provide for herein is a peptide display system, wherein the display system comprises: (a) one or more display surfaces; and (b) at least one distinct peptide, wherein the at least one distinct peptides comprises a binding motif, wherein the binding motif is selected from a motif group consisting of: RXX[CI]EXX[LI]F (SEQ ID NO:4), RXXQE[DN]XXF (SEQ ID NO:5), [MLIV]EXXLFXR (SEQ ID NO:6), D[ML]DXFXK (SEQ ID NO:7), FXXVC[ENA]K (SEQ ID NO:8), HGWTK (SEQ ID NO:9), [ED]XX[LM][TS][AC]XK (SEQ ID NO:10), D[PA]X[ML]X[STA]XK (SEQ ID NO:11), MT[TA][AS][KA] (SEQ ID NO:12), M[TS][ACT]AK (SEQ ID NO:13), EGXLXMXK (SEQ ID NO:14), FXXELXX[WY]F (SEQ ID NO: 15), [FY]XX[QE]LXXWF (SEQ ID NO: 16), QX[PT]C[LM]XK (SEQ ID NO: 17), QXP[MQ]LXK (SEQ ID NO:18), QXXC[LM]XKXXF (SEQ ID NO:19), CXXXPCMXK (SEQ ID NO:20), KXQTX[ST]M (SEQ ID NO:21), QX[AS]MXPK (SEQ ID NO:22), [TS]PMXXAXXK (SEQ ID NO:23), STXXL[TS]XK (SEQ ID NO:24), QX[SN]FKXX[FL](SEQ ID NO:25), RXPX[TS]L[ED] (SEQ ID NO:26), P[EDT]WRXY (SEQ ID NO:27), PXWR[VI]Y (SEQ ID NO:28), DXSGIXXX[DGESN] (SEQ ID NO:29), [APC]XXXDXSGI (SEQ ID NO:30), KTPP[LMI] (SEQ ID NO:31), LPFQQ (SEQ ID NO:32), K[FY][LM]XFXQ (SEQ ID NO:33), KWXXFQQ (SEQ ID NO:34), [CA]K[FY]XXFXQ (SEQ ID NO:35), CXXFIAXC (SEQ ID NO:36), CXXYWX[CA]XXK (SEQ ID NO:37), [LMI]LPXXXLD (SEQ ID NO:38), KXXGPXXLY (SEQ ID NO:39), QGPT[LI] (SEQ ID NO:40), KXG[TCA]PXXXXP (SEQ ID NO:41), WX[RK]A[DV]S (SEQ ID NO:42), [WTHN]XXXRA[VA]S (SEQ ID NO:43), [FY][RK]A[DAV][TS] (SEQ ID NO:44), CXXC[KR]AXS (SEQ ID NO:45), LX[AP]R[RKA]D (SEQ ID NO:46), FX[RK][RK]DT (SEQ ID NO:47), [RK][RKA]D[CTS]N (SEQ ID NO:48), [YF][RK]R[DA]XXX[PVR] (SEQ ID NO:49), WX[RK]MAD (SEQ ID NO:50), RMADXXR (SEQ ID NO:51), PQXXXDK[TSR](SEQ ID NO:52), [IV]XXX[QR][DN]KXH (SEQ ID NO:53), D[STR]NG[TS] (SEQ ID NO:54), D[SKH]NGT (SEQ ID NO:55), [VI]DXXG[TS][IL] (SEQ ID NO:56), DXXG[TS][FWY][MV](SEQ ID NO:57), [AI]DXXGTX[CHT] (SEQ ID NO:58), CXXDXXG[ST]C (SEQ ID NO:59), DXNX[TS]XK[YF] (SEQ ID NO:60), PNG[TS]XXP (SEQ ID NO:61), GXVDWD (SEQ ID NO:62), CXPXCXEXXW (SEQ ID NO:63), [AT]RA[VL]X[NTE] (SEQ ID NO:64), and wherein the at least one distinct peptide extends from the one or more display surfaces.

Also provided for herein is a peptide display system, wherein the display system comprises: (a) one or more display surfaces; and (b) at least one distinct peptide, wherein the at least one distinct peptides comprises a binding motif, wherein the binding motif is selected from a motif group consisting of: [AC]K[FY]XXFXQ (SEQ ID NO:73), [ACP]XXXDXSGI (SEQ ID NO:96), [AI]DXXGTX[CHT] (SEQ ID NO:58), [AIV]XXNG[ST]X[FLW] (SEQ ID NO:190), [AP][KR]AXSX[DENS] (SEQ ID NO:174), [AP]Q[EG]XTXXK (SEQ ID NO:97), [AS][KR]MAXS (SEQ ID NO:159), [AS]AX[FY]MXK (SEQ ID NO:134), [AT]RA[VL]X[NTE] (SEQ ID NO:64), [AT]XAVSXXX[HNY] (SEQ ID NO:170), [DE]CX[AGM]XKXC (SEQ ID NO:109), [DE]GXLX[LM]XK (SEQ ID NO:119), [DE]PX[LM]X[ACS]XK (SEQ ID NO: 122), [DN]AAXAL (SEQ ID NO:201), [ED]XX[LM][TS][AC]XK (SEQ ID NO:10), [EFY]TX[PS]LG (SEQ ID NO:84), [FI]AXEXDK (SEQ ID NO:200), [FL][DN]XY[FY]XD (SEQ ID NO:113), [FW][FY]XAXS[FY] (SEQ ID NO:160), [FW]XRRDXG (SEQ ID NO:182), [FY][KR][KR]DXXXP (SEQ ID NO:185), [FY][RK]A[DAV][TS] (SEQ ID NO:44), [FY]PXTXPK (SEQ ID NO:105), [FY]X[LV]LXXLN (SEQ ID NO:102), [FY]XKAXSXXY (SEQ ID NO:178), [FY]XRAMS (SEQ ID NO:180), [FY]XX[QE]LXXWF (SEQ ID NO:16), [GS][AS]AXXX[KR]K (SEQ ID NO:202), [GST]PXQYXD (SEQ ID NO:115), [HQ]X[AST][KR]AXS (SEQ ID NO:171), [ILM]LPXXXLD (SEQ ID NO:107), [ILMV]EXXLFXR (SEQ ID NO:92), [IMN][LM]X[KR]AXS (SEQ ID NO:177), [IR][FNW][KR]AXS (SEQ ID NO:168), [ITV]P[ST]WR (SEQ ID NO:81), [ITV]XXNG[ST][ILV] (SEQ ID NO:191), [IV]XXX[QR][DN]KXH (SEQ ID NO:53), [KR][AKR]D[CST]N (SEQ ID NO:187), [KR][KR]DCXXXC (SEQ ID NO:186), [KR]AVS[DEGNQ] (SEQ ID NO:161), [KR]AVXX[GR]X[FY] (SEQ ID NO:165), [KR]AXSMXXR (SEQ ID NO:175), [KR]AXSX[LM][DE] (SEQ ID NO:179), [KR]AXSXX[RW]M (SEQ ID NO: 167), [KR]AXSXX[ST]X[HRS] (SEQ ID NO:166), [KR]AXSXXX[HP][HY] (SEQ ID NO:176), [LM]X[HR]LNX[ILM] (SEQ ID NO:103), [NST]P[FM]XXX[ST]XK (SEQ ID NO:131), [RK][RKA]D[CTS]N (SEQ ID NO:48), [RK]S[ML]AS (SEQ ID NO: 199), [RY]ADXNG (SEQ ID NO: 189), [ST]PMXXAXXK (SEQ ID NO:132), [VI]DXXG[TS][IL] (SEQ ID NO:56), [WTHN]XXXRA[VA]S (SEQ ID NO:43), [YF][RK]R[DA]XXX[PVR] (SEQ ID NO:49), A[FIY]SG[GHS] (SEQ ID NO:197), AVSX[MNQ][HL] (SEQ ID NO:163), AXX[MS]RKP (SEQ ID NO:149), CSTIXC (SEQ ID NO:196), CXEX[ILM]LF (SEQ ID NO:147), CXLXWSXC (SEQ ID NO:142), CXVXPXMXK (SEQ ID NO:153), CXXC[KR]AXS (SEQ ID NO:45), CXXDXXG[ST]C (SEQ ID NO:59), CXXFIAXC (SEQ ID NO:36), CXXXPCMXK (SEQ ID NO:20), CXXXXCVMXK (SEQ ID NO:116), CXXYWX[AC]XXK (SEQ ID NO:152), D[LM]DXFXK (SEQ ID NO:108), D[LMY]SGI (SEQ ID NO:95), D[MR]NX[ST][LW] (SEQ ID NO:192), D[PA]X[ML]X[STA]XK (SEQ ID NO:11), D[SKH]NGT (SEQ ID NO:55), D[STR]NG[TS] (SEQ ID NO:54), DSNG[ST] (SEQ ID NO:188), DX[AC][GM][AFS]K (SEQ ID NO:110), DXAGXK (SEQ ID NO:111), DXNX[ST][CY][TW] (SEQ ID NO: 193), DXNX[TS]XK[YF] (SEQ ID NO:60), DXSGIXXX[DGESN] (SEQ ID NO:29), DXXG[TS][FWY][MV] (SEQ ID NO:57), EGXLXMXK (SEQ ID NO:14), ETXS[LM]G (SEQ ID NO:85), FP[AP][MT]XXK (SEQ ID NO:104), FX[RK][RK]DT (SEQ ID NO:47), FXE[HR]XK[PR] (SEQ ID NO: 146), FXQVCXK (SEQ ID NO:118), FXR[AK]DXXXP (SEQ ID NO:184), FXXELXX[WY]F (SEQ ID NO:15), FXXV[MT]RK (SEQ ID NO:117), FXXVC[ENA]K (SEQ ID NO:8), HGWTK (SEQ ID NO:9), HXQCPXXXK (SEQ ID NO:99), IPGS[FY] (SEQ ID NO:195), IXXAVSX[AS] (SEQ ID NO:172), K[DE]X[IV]PXXD (SEQ ID NO: 140), K[DS]G[ILV]XW (SEQ ID NO:203), K[FY][LM]XFXQ (SEQ ID NO:33), K[WY]X[AP]FXQ (SEQ ID NO:72), KAXSXXXXXQ (SEQ ID NO:169), KAXSXXXY (SEQ ID NO:173), KEXLXXWF (SEQ ID NO:93), KMXPWXQ (SEQ ID NO:141), KTPP[ILM] (SEQ ID NO:86), KWXXFQQ (SEQ ID NO:34), KXG[ACT]PXXXXP (SEQ ID NO:150), KXP[FW]CP (SEQ ID NO:155), KXQTX[ST]M (SEQ ID NO:21), KXSXIEX[ILM] (SEQ ID NO:91), KXX[AG]XQQ[FY] (SEQ ID NO:71), KXX[IV]Q[FY]XD (SEQ ID NO:114), KXXGPXXLY (SEQ ID NO:39), KXXIPYXD (SEQ ID NO:139), LLPXXX[IL]D (SEQ ID NO:106), LPFQQ (SEQ ID NO:32), LX[AP]R[RKA]D (SEQ ID NO:46), LX[IL]LXXLN (SEQ ID NO: 101), M[ST][ACT]AK (SEQ ID NO:123), MT[AT][AS][AK] (SEQ ID NO:124), P[EDT]WRXY (SEQ ID NO:27), PNG[TS]XXP (SEQ ID NO:61), PQXXXDK[TSR] (SEQ ID NO:52), PX[ANP][QR]XDK (SEQ ID NO:194), PXWR[IV]Y (SEQ ID NO:79), PXWXXG[LM]D (SEQ ID NO:148), PXXRAXSXX[HPW](SEQ ID NO:162), PXXXRAXSXG (SEQ ID NO:164), QGPT[LI] (SEQ ID NO:40), QGPTXP (SEQ ID NO:98), QX[AS]MXPK (SEQ ID NO:22), QX[NS]FKXX[FL] (SEQ ID NO:137), QX[PT]C[LM]XK (SEQ ID NO:17), QXP[MQ]LXK (SEQ ID NO:18), QXSFKXX[FMW](SEQ ID NO:136), QXXC[LM]XKXXF (SEQ ID NO:19), R[EHS]XXEXXLF (SEQ ID NO:87), R[FH]PEK (SEQ ID NO:145), RD[ST]XXPXR (SEQ ID NO:198), RMADX[AHR](SEQ ID NO:157), RMADXXR (SEQ ID NO:51), RS[ILMV]AS (SEQ ID NO:82), RXPX[ST]L[DE] (SEQ ID NO:77), RXPX[ST]LE (SEQ ID NO:78), RXX[CI]EXX[IL]F (SEQ ID NO:89), RXXQE[DN]XXF (SEQ ID NO:5), RXXQEXXLF (SEQ ID NO:88), SAXY[AMS]XK (SEQ ID NO:135), STXXL[ST]XK (SEQ ID NO: 130), SXXEDXLF (SEQ ID NO:90), TPMXXX[ST]XK (SEQ ID NO:129), WAXMAD (SEQ ID NO:158), WF[DE]XID (SEQ ID NO:144), WWXXL[LMT]XK (SEQ ID NO:120), WX[KR][AKR]D[ST] (SEQ ID NO:183), WX[RK]A[DV]S (SEQ ID NO:42), WX[RK]MAD (SEQ ID NO:50), WXX[IL]M[AL]K (SEQ ID NO:121), Y[KR]XD[ST][NY] (SEQ ID NO:181), YCXAXKXC (SEQ ID NO:154), YWXYFXK (SEQ ID NO:151), YX[IR][FY]ML (SEQ ID NO:143), and wherein the at least one distinct peptide extends from the one or more display surfaces.

Also provided for herein is a peptide display system, wherein the display system comprises: (a) one or more display surfaces; and (b) at least one distinct peptide, wherein the at least one distinct peptides comprises a binding motif, wherein the binding motif is selected from a motif group consisting of: [AP]Q[EG]XTXXK (SEQ ID NO:97), [AS]AX[FY]MXK (SEQ ID NO:134), [DE]CX[AGM]XKXC (SEQ ID NO:109), [DE]PX[LM]X[ACS]XK (SEQ ID NO:122), [EFY]TX[PS]LG (SEQ ID NO:84), [FL][DN]XY[FY]XD (SEQ ID NO:113), CXVXPXMXK (SEQ ID NO:153), CXXXXCVMXK (SEQ ID NO:116), D[LMY]SGI (SEQ ID NO:95), EGXLXMXK (SEQ ID NO:14), FP[AP][MT]XXK (SEQ ID NO:104), FXXELXX[WY]F (SEQ ID NO: 15), KXX[AG]XQQ[FY] (SEQ ID NO:71), KXXIPYXD (SEQ ID NO:139), LLPXXX[IL]D (SEQ ID NO:106), LX[IL]LXXLN (SEQ ID NO:101), PXWR[IV]Y (SEQ ID NO:79), QXSFKXX[FMW] (SEQ ID NO:136), R[EHS]XXEXXLF (SEQ ID NO:87), RS[ILMV]AS (SEQ ID NO:82), RXPX[ST]L[DE] (SEQ ID NO:77), TPMXXX[ST]XK (SEQ ID NO:129), WWXXL[LMT]XK (SEQ ID NO:120), [AC]K[FY]XXFXQ (SEQ ID NO:73), [ACP]XXXDXSGI (SEQ ID NO:96), [DE]GXLX[LM]XK (SEQ ID NO:119), [DN]AAXAL (SEQ ID NO:201), [ED]XX[LM][TS][AC]XK (SEQ ID NO:10), [FI]AXEXDK (SEQ ID NO:200), [FY]PXTXPK (SEQ ID NO:105), [FY]X[LV]LXXLN (SEQ ID NO:102), [FY]XX[QE]LXXWF (SEQ ID NO:16), [GS][AS]AXXX[KR]K (SEQ ID NO:202), [GST]PXQYXD (SEQ ID NO:115), [ILM]LPXXXLD (SEQ ID NO:107), [ILMV]EXXLFXR (SEQ ID NO:92), [ITV]P[ST]WR (SEQ ID NO:81), [LM]X[HR]LNX[ILM] (SEQ ID NO:103), [NST]P[FM]XXX[ST]XK (SEQ ID NO:131), [RK]S[ML]AS (SEQ ID NO: 199), [ST]PMXXAXXK (SEQ ID NO:132), AXX[MS]RKP (SEQ ID NO:149), CXEX[ILM]LF (SEQ ID NO:147), CXLXWSXC (SEQ ID NO:142), CXXFIAXC (SEQ ID NO:36), CXXXPCMXK (SEQ ID NO:20), CXXYWX[AC]XXK (SEQ ID NO: 152), D[LM]DXFXK (SEQ ID NO:108), D[PA]X[ML]X[STA]XK (SEQ ID NO:11), DX[AC][GM][AFS]K (SEQ ID NO:110), DXAGXK (SEQ ID NO:111), DXSGIXXX[DGESN] (SEQ ID NO:29), ETXS[LM]G (SEQ ID NO:85), FXE[HR]XK[PR] (SEQ ID NO:146), FXQVCXK (SEQ ID NO:118), FXXV[MT]RK (SEQ ID NO:117), FXXVC[ENA]K (SEQ ID NO:8), HGWTK (SEQ ID NO:9), HXQCPXXXK (SEQ ID NO:99), K[DE]X[IV]PXXD (SEQ ID NO:140), K[DS]G[ILV]XW (SEQ ID NO:203), K[FY][LM]XFXQ (SEQ ID NO:33), K[WY]X[AP]FXQ (SEQ ID NO:72), KEXLXXWF (SEQ ID NO:93), KMXPWXQ (SEQ ID NO:141), KTPP[ILM] (SEQ ID NO:86), KWXXFQQ (SEQ ID NO:34), KXG[ACT]PXXXXP (SEQ ID NO:150), KXP[FW]CP (SEQ ID NO:155), KXQTX[ST]M (SEQ ID NO:21), KXSXIEX[ILM] (SEQ ID NO:91), KXX[IV]Q[FY]XD (SEQ ID NO: 114), KXXGPXXLY (SEQ ID NO:39), LPFQQ (SEQ ID NO:32), M[ST][ACT]AK (SEQ ID NO:123), MT[AT][AS][AK] (SEQ ID NO: 124), P[EDT]WRXY (SEQ ID NO:27), PXWXXG[LM]D (SEQ ID NO:148), QGPT[LI] (SEQ ID NO:40), QGPTXP (SEQ ID NO:98), QX[AS]MXPK (SEQ ID NO:22), QX[NS]FKXX[FL] (SEQ ID NO:137), QX[PT]C[LM]XK (SEQ ID NO:17), QXP[MQ]LXK (SEQ ID NO:18), QXXC[LM]XKXXF (SEQ ID NO:19), R[FH]PEK (SEQ ID NO:145), RXPX[ST]LE (SEQ ID NO:78), RXX[CI]EXX[IL]F (SEQ ID NO:89), RXXQE[DN]XXF (SEQ ID NO:5), RXXQEXXLF (SEQ ID NO:88), SAXY[AMS]XK (SEQ ID NO:135), STXXL[ST]XK (SEQ ID NO:130), SXXEDXLF (SEQ ID NO:90), WF[DE]XID (SEQ ID NO:144), WXX[IL]M[AL]K (SEQ ID NO:121), YCXAXKXC (SEQ ID NO:154), YWXYFXK (SEQ ID NO:151), YX[IR][FY]ML (SEQ ID NO:143), and wherein the at least one distinct peptide extends from the one or more display surfaces.

Also provided for herein is a peptide display system, wherein the display system comprises: (a) one or more display surfaces; and (b) at least one distinct peptide, wherein the at least one distinct peptides comprises a binding motif, wherein the binding motif is selected from a motif group consisting of: PXWR[IV]Y (SEQ ID NO:79), P[EDT]WRXY (SEQ ID NO:27), [ITV]P[ST]WR (SEQ ID NO:81), [EFY]TX[PS]LG (SEQ ID NO:84), ETXS[LM]G (SEQ ID NO:85), KTPP[ILM] (SEQ ID NO:86), D[LMY]SGI (SEQ ID NO:95), [ACP]XXXDXSGI (SEQ ID NO:96), K[DS]G[ILV]XW (SEQ ID NO:203), [AP]Q[EG]XTXXK (SEQ ID NO:97), QGPTXP (SEQ ID NO:98), HXQCPXXXK (SEQ ID NO:99), QXP[MQ]LXK (SEQ ID NO:18), LX[IL]LXXLN (SEQ ID NO:101), [FY]X[LV]LXXLN (SEQ ID NO:102), [GS][AS]AXXX[KR]K (SEQ ID NO:202), FP[AP][MT]XXK (SEQ ID NO:104), [FY]PXTXPK (SEQ ID NO:105), KXQTX[ST]M (SEQ ID NO:21), [ILM]LPXXXLD (SEQ ID NO:107), [FL][DN]XY[FY]XD (SEQ ID NO:113), KXX[IV]Q[FY]XD (SEQ ID NO:114), FXXV[MT]RK (SEQ ID NO:117), FXQVCXK (SEQ ID NO:118), FXXVC[ENA]K (SEQ ID NO:8), EGXLXMXK (SEQ ID NO:14), [DE]GXLX[LM]XK (SEQ ID NO:119), WWXXL[LMT]XK (SEQ ID NO:120), WXX[IL]M[AL]K (SEQ ID NO:121), [DE]PX[LM]X[ACS]XK (SEQ ID NO: 122), M[ST][ACT]AK (SEQ ID NO:123), MT[AT][AS][AK] (SEQ ID NO:124), [ED]XX[LM][TS][AC]XK (SEQ ID NO: 10), D[PA]X[ML]X[STA]XK (SEQ ID NO:11), TPMXXX[ST]XK (SEQ ID NO: 129), STXXL[ST]XK (SEQ ID NO:130), [NST]P[FM]XXX[ST]XK (SEQ ID NO:131), [ST]PMXXAXXK (SEQ ID NO:132), [AS]AX[FY]MXK (SEQ ID NO:134), SAXY[AMS]XK (SEQ ID NO:135), QXSFKXX[FMW] (SEQ ID NO:136), QX[NS]FKXX[FL] (SEQ ID NO:137), K[DE]X[IV]PXXD (SEQ ID NO:140), KMXPWXQ (SEQ ID NO:141), CXLXWSXC (SEQ ID NO:142), YX[IR][FY]ML (SEQ ID NO:143), WF[DE]XID (SEQ ID NO:144), R[FH]PEK (SEQ ID NO:145), FXE[HR]XK[PR] (SEQ ID NO:146), PXWXXG[LM]D (SEQ ID NO:148), AXX[MS]RKP (SEQ ID NO:149), CXXFIAXC (SEQ ID NO:36), HGWTK (SEQ ID NO:9), KXG[ACT]PXXXXP (SEQ ID NO:150), YWXYFXK (SEQ ID NO:151), CXXYWX[AC]XXK (SEQ ID NO:152), CXVXPXMXK (SEQ ID NO:153), CXXXPCMXK (SEQ ID NO:20), YCXAXKXC (SEQ ID NO:154), KXXGPXXLY (SEQ ID NO:39), and wherein the at least one distinct peptide extends from the one or more display surfaces.

Also provided for herein is a peptide display system, wherein the display system comprises: (a) one or more display surfaces; and (b) at least one distinct peptide, wherein the at least one distinct peptides comprises a binding motif, wherein the binding motif is selected from a motif group consisting of: [AS]AX[FY]MXK (SEQ ID NO:134), [DE]PX[LM]X[ACS]XK (SEQ ID NO:122), HGWTK (SEQ ID NO:9), R[FH]PEK (SEQ ID NO:145), [AP]Q[EG]XTXXK (SEQ ID NO:97), [FL][DN]XY[FY]XD (SEQ ID NO:113), AXX[MS]RKP (SEQ ID NO:149), D[LM]DXFXK (SEQ ID NO:108), EGXLXMXK (SEQ ID NO:14), KXQTX[ST]M (SEQ ID NO:21), KXXGPXXLY (SEQ ID NO:39), LX[IL]LXXLN (SEQ ID NO:101), IPGS[FY] (SEQ ID NO:195), A[FIY]SG[GHS] (SEQ ID NO:197), and wherein the at least one distinct peptide extends from the one or more display surfaces.

In some aspects, the display system comprises at least 2 distinct peptides each comprising a distinct binding motif, wherein each distinct binding motif is selected from the motif group. In some aspects, the display system comprises at least 3, at least 4, or at least 5 distinct peptides each comprising a distinct binding motif, wherein each distinct binding motif is selected from the motif group. In some aspects, the display system comprises at least 6, at least 7, at least 8, or at least 9 distinct peptides each comprising a distinct binding motif, wherein each distinct binding motif is selected from the motif group. In some aspects, the display system comprises at least 10, at least 15, at least 20, at least 25, at least 30, at least 35, at least 40, at least 45, at least 50, at least 55, or at least 60 distinct peptides each comprising a distinct binding motif, wherein each distinct binding motif is selected from the motif group.

In some aspects, wherein the at least one distinct peptide is capable of binding to an antibody associated with a SARS-CoV-2 infection.

In some aspects, the one or more display surfaces comprises a single display surface. In some aspects, the single display surface comprises a solid support. In some aspects, each of the at least one distinct peptides is attached at a defined location on the single display surface. In some aspects, the single display surface is a peptide microarray. In some aspects, the defined location on the peptide microarray is a defined grid location on the peptide microarray. In some aspects, the single display surface is a multi-well plate. In some aspects, the defined location in the multi-well plate is a defined well in the multi-well plate. In some aspects, the single display surface comprises a lateral flow assay. In some aspects, the defined location in the lateral flow assay is a defined location on a capillary membrane.

In some aspects, the one or more display surfaces comprises a plurality of display surfaces. In some aspects, the plurality of display surfaces is a plurality of biological entity surfaces. In some aspects, the biological entity comprises a cell, a virus, or a bacteriophage. In some aspects, the at least one distinct peptide is encoded by one or more distinct nucleic acid sequences capable of expressing the at least one distinct peptide in the plurality of biological entities. In some aspects, the at least one distinct peptide comprises a surface display peptide sequence configured to be expressed as part of the peptide and capable of directing the peptides for display on the biological entity surface. In some aspects, the biological entity is an Escherichia coli cell. In some aspects, the at least one distinct peptide comprises at least a portion of an Escherichia coli eCPX scaffold. In some aspects, the plurality of display surfaces is a plurality of microparticle surfaces. In some aspects, each of the plurality of display surfaces comprises a distinct single species of the at least one distinct peptides.

In some aspects, the at least one distinct peptide further comprises a label.

In some aspects, the one or more display surfaces or the distinct peptides is bound or capable of being bound directly or indirectly to a capture entity. In some aspects, the capture entity comprises a solid support selected from the group consisting of: an agarose bead, a sepharose bead, a magnetic bead, and a resin. In some aspects, the capture entity comprises a binding moiety that binds directly to the one or more display surfaces or the distinct peptides. In some aspects, the capture entity comprises a binding moiety that binds a binding molecule that binds to the one or more display surfaces or at least one of the distinct peptides. In some aspects, the binding molecule that binds to at least one of the distinct peptides is an antibody from a biological sample. In some aspects, the binding moiety is selected from the group consisting of: Protein A, Protein G, Protein A/G, and an antibody or antigen binding fragment thereof. In some aspects, the antibody or antigen binding fragment thereof comprises an anti-immunoglobulin antibody. In some aspects, the binding moiety comprises streptavidin and the one or more display surfaces, the distinct peptide, or the binding molecule that binds to the one or more display surfaces or at least one of the distinct peptides further comprises biotin. In some aspects, the capture entity further comprises a fluorophore.

In some aspects, displaying comprises presentation of each peptide for direct binding by a binding molecule. In some aspects, the binding molecule is selected from the group consisting of: an antibody or antigen-binding fragment thereof, an aptamer, a peptoid, and an affibody. In some aspects, the binding molecule is an antibody or antigen-binding fragment thereof.

In some aspects, the composition further comprises a library of peptides. In some aspects, the library of peptides is a random peptide library. In some aspects, the library of peptides is a non-random peptide library. In some aspects, the non-random library is biased to represent one or more diseases or conditions, and wherein at least one of the one or more diseases or conditions is a SARS-CoV-2 infection. In some aspects, the biased representation is at least greater than a representation in a random peptide library. In some aspects, the greater representation is statistically significant, wherein the statistical significance has a p-value less than 0.05. In some aspects, each of the peptides in the library of peptides is at least 5, 6, 7, 8, 9, 10, 11, 12, 13, 14, 15, 16, 17, 18, 19, or 20 amino acids in length.

In some aspects, each of the distinct peptides and each of the peptides in the library of peptides is an identical defined length. In some aspects, the identical defined length is 5, 6, 7, 8, 9, 10, 11, 12, 13, 14, 15, 16, 17, 18, 19, or 20 amino acids in length. In some aspects, the identical defined length is 12 amino acids in length.

In some aspects, the diversity of the library is less than 8×10⁹ distinct peptides. In some aspects, the diversity of the library is 384 or fewer, 96 or fewer, 48 or fewer, 24 or fewer, or 12 or fewer distinct peptides. In some aspects, the diversity of the library is at least 10, at least 100, at least 1000, at least 10⁴, at least 10⁵, at least 10⁶, at least 10⁷, at least 10⁸ distinct peptides.

In some aspects, the binding motifs are selected from IgG isotype specific binding motifs. In some aspects, the binding motifs are selected from IgM isotype specific binding motifs.

Also provide for herein is a method of determining the presence of specimen antibodies specific for SARS-CoV-2 in a biological sample comprising: contacting any of the display systems provided herein with a biological sample, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the at least one distinct peptide and the specimen antibodies; and determining specimen antibodies specific for SARS-CoV-2 are present in the biological sample when binding between at least one of the at least one distinct peptides and the specimen antibodies is detected.

Also provide for herein is a method of treating a subject known or suspected of suffering from a SARS-CoV-2 infection comprising assessing or having assessed whether the subject suffers from a SARS-CoV-2 infection, wherein the assessment comprises: contacting any of the display systems provided herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the at least one distinct peptide and the specimen antibodies; and assessing the subject as: i) positive for the SARS-CoV-2 infection, or

ii) negative for the SARS-CoV-2 infection; and administering or having administered a therapy to treat the SARS-CoV-2 infection when the subject when the subject is assessed as positive.

Also provide for herein is a method of determining whether a subject is a candidate for treatment of a SARS-CoV-2 infection comprising assessing or having assessed whether the subject suffers from a SARS-CoV-2 infection, wherein the assessment comprises: contacting any of the display systems provided herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the at least one distinct peptide and the specimen antibodies; and diagnosing the subject as: i) positive for the SARS-CoV-2 infection, or ii) negative for the SARS-CoV-2 infection, determining the subject is a candidate for a treatment of the SARS-CoV-2 infection when the subject is assessed as positive.

Also provide for herein is a method of diagnosing a SARS-CoV-2 infection in a subject comprising: contacting any of the display systems provided herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the at least one distinct peptide and the specimen antibodies; and diagnosing the subject as: i) positive for the SARS-CoV-2 infection, or ii) negative for the SARS-CoV-2 infection.

In some aspects, the assessing or diagnosing comprises assessing or diagnosing the subject as: i) positive for the SARS-CoV-2 infection when binding between at least one of the at least one distinct peptides and the specimen antibodies is detected, or ii) negative for the SARS-CoV-2 infection when binding between less than at least one of the at least one distinct peptides and the specimen antibodies is detected. In some aspects, the assessing or diagnosing comprises assessing or diagnosing the subject as: i) positive for the SARS-CoV-2 infection when binding between at least 2, at least 3, at least 4, or at least 5 distinct peptides of the at least one distinct peptides and the specimen antibodies is detected, or ii) negative for the SARS-CoV-2 infection when binding between less than at least 2, at least 3, at least 4, or at least 5 distinct peptides, respectively, of the at least one distinct peptides and the specimen antibodies is detected. In some aspects, the assessing or diagnosing comprises assessing or diagnosing the subject as: i) positive for the SARS-CoV-2 infection when binding between at least 6, at least 7, at least 8, at least 9, or at least 10 distinct peptides of the at least one distinct peptides and the specimen antibodies is detected, or ii) negative for the SARS-CoV-2 infection when binding between less than at least 6, at least 7, at least 8, at least 9, or at least 10 distinct peptides, respectively, of the at least one distinct peptides and the specimen antibodies is detected. In some aspects, the measuring comprises calculating a z-score for binding between the at least one distinct peptide and the specimen antibodies and the assessing or diagnosing comprises assessing or diagnosing the subject as: i) positive for the SARS-CoV-2 infection when the z-score meets or exceeds a threshold value, or ii) negative for the SARS-CoV-2 infection when the z-score is less than the threshold value.

In some aspects, the method further comprises assessing severity of the SARS-CoV-2 infection in the subject. In some aspects, assessing severity of the SARS-CoV-2 infection comprises: measuring the binding between the specimen antibodies and one or more distinct peptides associated with severe and/or moderate Covid disease; and diagnosing or assessing the infection as mild, moderate, or severe disease.

Also provided for herein is a method of assessing disease severity of a SARS-CoV-2 infection in a subject comprising: contacting any one of the display systems described herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the specimen antibodies and one or more distinct peptides associated with severe and/or moderate Covid disease; and diagnosing or assessing the infection as mild, moderate, or severe disease.

In some aspects, the method further comprises contacting the biological sample with at least one reagent configured to remove antibodies that bind to array components other than the at least one distinct peptide before contacting the display system of with the biological sample from the subject.

In some aspects, the at least one reagent comprises the one or more display surfaces free of the at least one distinct peptide.

In some aspects, the measuring comprises use of an assay selected from the group consisting of: optical scanning, an ELISA, fluorescent detection, digital serology, flow-cytometry, surface plasmon resonance, bio-layer interferometry, lateral flow assay, or combinations thereof.

In some aspects, the method further comprises determining amino acid sequences of peptides in the display system bound by the specimen antibodies. In some aspects, the method further comprises determining binding motifs of peptides in the display system bound by the specimen antibodies using a clustering algorithm. In some aspects, the method further comprises determining the isotype of the specimen antibodies that bind to the peptides in the display system. In some aspects, the method further comprises determining the isotype of the specimen antibodies that bind to the at least one distinct peptide. In some aspects, the method further comprises prior to the measuring step isolating specific specimen antibody isotypes. In some aspects, the measuring step further comprises measuring the binding between the at least one distinct peptide and specific specimen antibody isotypes. In some aspects, the isotype is IgM. In some aspects, the isotype is IgG or subclasses thereof.

In some aspects, calculating the z-score comprises calculating a composite z-score for binding between each of the at least one distinct peptides specific for SARS-CoV-2 and the specimen antibodies.

In some aspects, the subject is known or suspected of having an active SARS-CoV-2 infection. In some aspects, the subject is known or suspected of having recovered from a SARS-CoV-2 infection. In some aspects, the subject was diagnosed as having a SARS-CoV-2 infection.

The method of any one of claims Error! Reference source not found.-Error! Reference source not found., wherein the biological sample from the subject was collected at least 6 days, at least 11 days, at least 16 days, or at least 20 after system onset.

In some aspects, the biological sample from the subject was collected between 1 and 5 days, between 6 and 10 days, between 11 and 15 days, between 16 and 20 days, or greater than 20 days after system onset.

In some aspects, the biological sample comprises a bodily fluid. In some aspects, the bodily fluid is peripheral blood, lymphatic fluid, sweat, saliva, or mucus.

In some aspects, the method has a sensitivity of at least 45%, at least 50%, at least 60%, at least 70%, at least 80%, at least 90%, or at least 95%. In some aspects, the method has a sensitivity of at least 52%, at least 53%, or at least 56%. In some aspects, the method has a sensitivity of at least 56%, at least 67%, or at least 76%. In some aspects, the method has a sensitivity of at least 50%, at least 73%, at least 75%, at least 88%, or at least 91%. In some aspects, the method has a sensitivity of at least 58%. In some aspects, the method has a sensitivity of at least 73%. In some aspects, the method has a sensitivity of at least 77%. In some aspects, the method has a sensitivity of at least 82%. In some aspects, the method has a sensitivity of at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, or at least 89%.

In some aspects, the method has a specificity of at least 90%, at least 91%, at least 92%, at least 93%, at least 94%, at least 95%, at least 96%, at least 97%, at least 98%, or at least 99%. In some aspects, the method has a specificity of at least 99%. In some aspects, the method has a sensitivity of at least 53% and a specificity of at least 99%. In some aspects, the method has a sensitivity of at least 73% and a specificity of at least 99%. In some aspects, the method has a sensitivity of at least 77% and a specificity of at least 99%. In some aspects, the method has a sensitivity of at least 82% and a specificity of at least 99%.

A kit for diagnosing a SARS-CoV-2 infection, comprising the display system of any of the above composition claims and a system for detecting the binding of each of the at least one distinct peptides to its target antibody. In some aspects, the kit further comprises an agent for removing antibodies that bind to array components other than the at least one distinct peptide.

Also provide for herein is a peptide expression library, wherein the library comprises a library of nucleic acid sequences encoding a library of peptides, wherein the library of peptides comprises at least one distinct peptide, wherein the at least one distinct peptide comprises a binding motif, wherein the binding motif is selected from a motif group consisting of:

(SEQ ID NO: 4)
RXX[CI]EXX[LI]F,
(SEQ ID NO: 5)
RXXQE[DN]XXF,
(SEQ ID NO: 6)
[MLIV]EXXLFXR,
(SEQ ID NO: 7)
D[ML]DXFXK,
(SEQ ID NO: 8)
FXXVC[ENA]K,
(SEQ ID NO: 9)
HGWTK,
(SEQ ID NO: 10)
[ED]XX[LM][TS][AC]XK,
(SEQ ID NO: 11)
D[PA]X[ML]X[STA]XK,
(SEQ ID NO: 12)
MT[TA][AS][KA],
(SEQ ID NO: 13)
M[TS][ACT]AK,
(SEQ ID NO: 14)
EGXLXMXK,
(SEQ ID NO: 15)
FXXELXX[WY]F,
(SEQ ID NO: 16)
[FY]XX[QE]LXXWF,
(SEQ ID NO: 17)
QX[PT]C[LM]XK,
(SEQ ID NO: 18)
QXP[MQ]LXK,
(SEQ ID NO: 19)
QXXC[LM]XKXXF,
(SEQ ID NO: 20)
CXXXPCMXK,
(SEQ ID NO: 21)
KXQTX[ST]M,
(SEQ ID NO: 22)
QX[AS]MXPK,
(SEQ ID NO: 23)
[TS]PMXXAXXK,
(SEQ ID NO: 24)
STXXL[TS]XK,
(SEQ ID NO: 25)
QX[SN]FKXX[FL],
(SEQ ID NO: 26)
RXPX[TS]L[ED],
(SEQ ID NO: 27)
P[EDT]WRXY,
(SEQ ID NO: 28)
PXWR[VI]Y,
(SEQ ID NO: 29)
DXSGIXXX[DGESN],
(SEQ ID NO: 30)
[APC]XXXDXSGI,
(SEQ ID NO: 31)
KTPP[LMI],
(SEQ ID NO: 32)
LPFQQ,
(SEQ ID NO: 33)
K[FY][LM]XFXQ,
(SEQ ID NO: 34)
KWXXFQQ,
(SEQ ID NO: 35)
[CA]K[FY]XXFXQ,
(SEQ ID NO: 36)
CXXFIAXC,
(SEQ ID NO: 37)
CXXYWX[CA]XXK,
(SEQ ID NO: 38)
[LMI]LPXXXLD,
(SEQ ID NO: 39)
KXXGPXXLY,
(SEQ ID NO: 40)
QGPT[LI],
(SEQ ID NO: 41)
KXG[TCA]PXXXXP,
(SEQ ID NO: 42)
WX[RK]A[DV]S,
(SEQ ID NO: 43)
[WTHN]XXXRA[VA]S,
(SEQ ID NO: 44)
[FY][RK]A[DAV][TS],
(SEQ ID NO: 45)
CXXC[KR]AXS,
(SEQ ID NO: 46)
LX[AP]R[RKA]D,
(SEQ ID NO: 47)
FX[RK][RK]DT,
(SEQ ID NO: 48)
[RK][RKA]D[CTS]N,
(SEQ ID NO: 49)
[YF][RK]R[DA]XXX[PVR],
(SEQ ID NO: 50)
WX[RK]MAD,
(SEQ ID NO: 51)
RMADXXR,
(SEQ ID NO: 52)
PQXXXDK[TSR],
(SEQ ID NO: 53)
[IV]XXX[QR][DN]KXH,
(SEQ ID NO: 54)
D[STRING[TS],
(SEQ ID NO: 55)
D[SKH]NGT,
(SEQ ID NO: 56)
[VI]DXXG[TS][IL],
(SEQ ID NO: 57)
DXXG[TS][FWY][MV],
(SEQ ID NO: 58)
[AI]DXXGTX[CHT],
(SEQ ID NO: 59)
CXXDXXG[ST]C,
(SEQ ID NO: 60)
DXNX[TS]XK[YF],
(SEQ ID NO: 61)
PNG[TS]XXP,
(SEQ ID NO: 62)
GXVDWD,
(SEQ ID NO: 63)
CXPXCXEXXW,
(SEQ ID NO: 64)
[AT]RA[VL]X[NTE].

Also provide for herein is a peptide expression library, wherein the library comprises a library of nucleic acid sequences encoding a library of peptides, wherein the library of peptides comprises at least one distinct peptide, wherein the at least one distinct peptide comprises a binding motif, wherein the binding motif is selected from a motif group consisting of:

(SEQ ID NO: 73)
[AC]K[FY]XXFXQ,
(SEQ ID NO: 96)
[ACP]XXXDXSGI,
(SEQ ID NO: 58)
[AT]DXXGTX[CHT],
(SEQ ID NO: 190)
[AIV]XXNG[ST]X[FLW],
(SEQ ID NO: 174)
[AP][KR]AXSX[DENS],
(SEQ ID NO: 97)
[AP]Q[EG]XTXXK,
(SEQ ID NO: 159)
[AS][KR]MAXS,
(SEQ ID NO: 134)
[AS]AX[FY]MXK,
(SEQ ID NO: 64)
[AT]RA[VL]X[NTE],
(SEQ ID NO: 170)
[AT]XAVSXXX[HNY],
(SEQ ID NO: 109)
[DE]CX[AGM]XKXC,
(SEQ ID NO: 119)
[DE]GXLX[LM]XK,
(SEQ ID NO: 122)
[DE]PX[LM]X[ACS]XK,
(SEQ ID NO: 201)
[DN]AAXAL,
(SEQ ID NO: 10)
[ED]XX[LM][TS][AC]XK,
(SEQ ID NO: 84)
[EFY]TX[PS]LG,
(SEQ ID NO: 200)
[FI]AXEXDK,
(SEQ ID NO: 113)
[FL][DN]XY[FY]XD,
(SEQ ID NO: 160)
[FW][FY]XAXS[FY],
(SEQ ID NO: 182)
[FW]XRRDXG,
(SEQ ID NO: 185)
[FY][KR][KR]DXXXP,
(SEQ ID NO: 44)
[FY][RK]A[DAV][TS],
(SEQ ID NO: 105)
[FY]PXTXPK,
(SEQ ID NO: 102)
[FY]X[LV]LXXLN,
(SEQ ID NO: 178)
[FY]XKAXSXXY,
(SEQ ID NO: 180)
[FY]XRAMS,
(SEQ ID NO: 16)
[FY]XX[QE]LXXWF,
(SEQ ID NO: 202)
[GS][AS]AXXX[KR]K,
(SEQ ID NO: 115)
[GST]PXQYXD,
(SEQ ID NO: 171)
[HQ]X[AST][KR]AXS,
(SEQ ID NO: 107)
[ILM]LPXXXLD,
(SEQ ID NO: 92)
[ILMV]EXXLFXR,
(SEQ ID NO: 177)
[IMN][LM]X|KR]AXS,
(SEQ ID NO: 168)
[IR][FNW][KR]AXS,
(SEQ ID NO: 81)
[ITV]P[ST]WR,
(SEQ ID NO: 191)
[ITV]XXNG[ST][ILV],
(SEQ ID NO: 53)
[IV]XXX[QR][DN]KXH,
(SEQ ID NO: 187)
[KR][AKR]D[CST]N,
(SEQ ID NO: 186)
[KR][KR]DCXXXC,
(SEQ ID NO: 161)
[KR]AVS[DEGNQ],
(SEQ ID NO: 165)
[KR]AVXX[GR]X[FY],
(SEQ ID NO: 175)
[KR]AXSMXXR,
(SEQ ID NO: 179)
[KR]AXSX[LM][DE],
(SEQ ID NO: 167)
[KR]AXSXX[RW]M,
(SEQ ID NO: 166)
[KR]AXSXX[ST]X[HRS],
(SEQ ID NO: 176)
[KR]AXSXXX[HP][HY],
(SEQ ID NO: 103)
[LM]X[HR]LNX[ILM],
(SEQ ID NO: 131)
[NST]P[FM]XXX[ST]XK,
(SEQ ID NO: 48)
[RK][RKA]D[CTS]N,
(SEQ ID NO: 199)
[RK]S[ML]AS,
(SEQ ID NO: 189)
[RY]ADXNG,
(SEQ ID NO: 132)
[ST]PMXXAXXK,
(SEQ ID NO: 56)
[VI]DXXG[TS][IL],
(SEQ ID NO: 43)
[WTHN]XXXRA[VA]S,
(SEQ ID NO: 49)
[YF][RK]R[DA]XXX[PVR],
(SEQ ID NO: 197)
A[FIY]SG[GHS],
(SEQ ID NO: 163)
AVSX[MNQ][HL],
(SEQ ID NO: 149)
AXX[MS]RKP,
(SEQ ID NO: 196)
CSTIXC,
(SEQ ID NO: 147)
CXEX[ILM]LF,
(SEQ ID NO: 142)
CXLXWSXC,
(SEQ ID NO: 153)
CXVXPXMXK,
(SEQ ID NO: 45)
CXXC[KR]AXS,
(SEQ ID NO: 59)
CXXDXXG[ST]C,
(SEQ ID NO: 36)
CXXFIAXC,
(SEQ ID NO: 20)
CXXXPCMXK,
(SEQ ID NO: 116)
CXXXXCVMXK,
(SEQ ID NO: 152)
CXXYWX[AC]XXK,
(SEQ ID NO: 108)
D[LM]DXFXK,
(SEQ ID NO: 95)
D[LMY]SGI,
(SEQ ID NO: 192)
D[MR]NX[ST][LW],
(SEQ ID NO: 11)
D[PA]X[ML]X[STA]XK,
(SEQ ID NO: 55)
D[SKH]NGT,
(SEQ ID NO: 54)
D[STRING[TS],
(SEQ ID NO: 188)
DSNG[ST],
(SEQ ID NO: 110)
DX[AC][GM][AFS]K,
(SEQ ID NO: 111)
DXAGXK,
(SEQ ID NO: 193)
DXNX[ST][CY][TW],
(SEQ ID NO: 60)
DXNX[TS]XK[YF],
(SEQ ID NO: 29)
DXSGIXXX[DGESN],
(SEQ ID NO: 57)
DXXG[TS][FWY][MV],
(SEQ ID NO: 14)
EGXLXMXK,
(SEQ ID NO: 85)
ETXS[LM]G,
(SEQ ID NO: 104)
FP[AP][MT]XXK,
(SEQ ID NO: 47)
FX[RK][RK]DT,
(SEQ ID NO: 146)
FXE[HR]XK[PR],
(SEQ ID NO: 118)
FXQVCXK,
(SEQ ID NO: 184)
FXR[AK]DXXXP,
(SEQ ID NO: 15)
FXXELXX[WY]F,
(SEQ ID NO: 117)
FXXV[MT]RK,
(SEQ ID NO: 8)
FXXVC[ENA]K,
(SEQ ID NO: 9)
HGWTK,
(SEQ ID NO: 99)
HXQCPXXXK,
(SEQ ID NO: 195)
IPGS[FY],
(SEQ ID NO: 172)
IXXAVSX[AS],
(SEQ ID NO: 140)
K[DE]X[IV]PXXD,
(SEQ ID NO: 203)
K[DS]G[ILV]XW,
(SEQ ID NO: 33)
K[FY][LM]XFXQ,
(SEQ ID NO: 72)
K[WY]X[AP]FXQ,
(SEQ ID NO: 169)
KAXSXXXXXQ,
(SEQ ID NO: 173)
KAXSXXXY,
(SEQ ID NO: 93)
KEXLXXWF,
(SEQ ID NO: 141)
KMXPWXQ,
(SEQ ID NO: 86)
KTPP[ILM],
(SEQ ID NO: 34)
KWXXFQQ,
(SEQ ID NO: 150)
KXG[ACT]PXXXXP,
(SEQ ID NO: 155)
KXP[FW]CP,
(SEQ ID NO: 21)
KXQTX[ST]M,
(SEQ ID NO: 91)
KXSXIEX[ILM],
(SEQ ID NO: 71)
KXX[AG]XQQ[FY],
(SEQ ID NO: 114)
KXX[IV]Q[FY]XD,
(SEQ ID NO: 39)
KXXGPXXLY,
(SEQ ID NO: 139)
KXXIPYXD,
(SEQ ID NO: 106)
LLPXXX[IL]D,
(SEQ ID NO: 32)
LPFQQ,
(SEQ ID NO: 46)
LX[AP]R[RKA]D,
(SEQ ID NO: 101)
LX|IL]LXXLN,
(SEQ ID NO: 123)
M[ST][ACT]AK,
(SEQ ID NO: 124)
MT[AT][AS][AK],
(SEQ ID NO: 27)
P[EDT]WRXY,
(SEQ ID NO: 61)
PNG[TS]XXP,
(SEQ ID NO: 52)
PQXXXDK[TSR],
(SEQ ID NO: 194)
PX[ANP][QR]XDK,
(SEQ ID NO: 79)
PXWR[IV]Y,
(SEQ ID NO: 148)
PXWXXG[LM]D,
(SEQ ID NO: 162)
PXXRAXSXX[HPW],
(SEQ ID NO: 164)
PXXXRAXSXG,
(SEQ ID NO: 40)
QGPT[LI],
(SEQ ID NO: 98)
QGPTXP,
(SEQ ID NO: 22)
QX[AS]MXPK,
(SEQ ID NO: 137)
QX[NS]FKXX[FL],
(SEQ ID NO: 17)
QX[PT]C[LM]XK,
(SEQ ID NO: 18)
QXP[MQ]LXK,
(SEQ ID NO: 136)
QXSFKXX[FMW],
(SEQ ID NO: 19)
QXXC[LM]XKXXF,
(SEQ ID NO: 87)
R[EHS]XXEXXLF,
(SEQ ID NO: 145)
R[FH]PEK,
(SEQ ID NO: 198)
RD[ST]XXPXR,
(SEQ ID NO: 157)
RMADX[AHR],
(SEQ ID NO: 51)
RMADXXR,
(SEQ ID NO: 82)
RS[ILMV]AS,
(SEQ ID NO: 77)
RXPX[ST]L[DE],
(SEQ ID NO: 78)
RXPX[ST]LE,
(SEQ ID NO: 89)
RXX[CI]EXX[IL]F,
(SEQ ID NO: 5)
RXXQE[DN]XXF,
(SEQ ID NO: 88)
RXXQEXXLF,
(SEQ ID NO: 135)
SAXY[AMS]XK,
(SEQ ID NO: 130)
STXXL[ST]XK,
(SEQ ID NO: 90)
SXXEDXLF,
(SEQ ID NO: 129)
TPMXXX[ST]XK,
(SEQ ID NO: 158)
WAXMAD,
(SEQ ID NO: 144)
WF[DE]XID,
(SEQ ID NO: 120)
WWXXL[LMT]XK,
(SEQ ID NO: 183)
WX[KR][AKR]D[ST],
(SEQ ID NO: 42)
WX[RK]A[DV]S,
(SEQ ID NO: 50)
WX[RK]MAD,
(SEQ ID NO: 121)
WXX[IL]M[AL]K,
(SEQ ID NO: 181)
Y[KR]XD[ST][NY],
(SEQ ID NO: 154)
YCXAXKXC,
(SEQ ID NO: 151)
YWXYFXK,
(SEQ ID NO: 143)
YX[IR][FY]ML .

Also provide for herein is a peptide expression library, wherein the library comprises a library of nucleic acid sequences encoding a library of peptides, wherein the library of peptides comprises at least one distinct peptide, wherein the at least one distinct peptide comprises a binding motif, wherein the binding motif is selected from a motif group consisting of:

(SEQ ID NO: 97)
[AP]Q[EG]XTXXK,
(SEQ ID NO: 134)
[AS]AX[FY]MXK,
(SEQ ID NO: 109)
[DE]CX[AGM]XKXC,
(SEQ ID NO: 122)
[DE]PX[LM]X[ACS]XK,
(SEQ ID NO: 84)
[EFY]TX[PS]LG,
(SEQ ID NO: 113)
[FL][DN]XY[FY]XD,
(SEQ ID NO: 153)
CXVXPXMXK,
(SEQ ID NO: 116)
CXXXXCVMXK,
(SEQ ID NO: 95)
D[LMY]SGI,
(SEQ ID NO: 14)
EGXLXMXK,
(SEQ ID NO: 104)
FP[AP][MT]XXK,
(SEQ ID NO: 15)
FXXELXX[WY]F,
(SEQ ID NO: 71)
KXX[AG]XQQ[FY],
(SEQ ID NO: 139)
KXXIPYXD,
(SEQ ID NO: 106)
LLPXXX[IL]D,
(SEQ ID NO: 101)
LX[IL]LXXLN,
(SEQ ID NO: 79)
PXWR[IV]Y,
(SEQ ID NO: 136)
QXSFKXX[FMW],
(SEQ ID NO: 87)
R[EHS]XXEXXLF,
(SEQ ID NO: 82)
RS[ILMV]AS,
(SEQ ID NO: 77)
RXPX[ST]L[DE],
(SEQ ID NO: 129)
TPMXXX[ST]XK,
(SEQ ID NO: 120)
WWXXL[LMT]XK,
(SEQ ID NO: 73)
[AC]K[FY]XXFXQ,
(SEQ ID NO: 96)
[ACP]XXXDXSGI,
(SEQ ID NO: 119)
[DE]GXLX[LM]XK,
(SEQ ID NO: 201)
[DN]AAXAL,
(SEQ ID NO: 10)
[ED]XX[LM][TS][AC]XK,
(SEQ ID NO: 200)
[FI]AXEXDK,
(SEQ ID NO: 105)
[FY]PXTXPK,
(SEQ ID NO: 102)
[FY]X[LV]LXXLN,
(SEQ ID NO: 16)
[FY]XX[QE]LXXWF,
(SEQ ID NO: 202)
[GS][AS]AXXX[KR]K,
(SEQ ID NO: 115)
[GST]PXQYXD,
(SEQ ID NO: 107)
[ILM]LPXXXLD,
(SEQ ID NO: 92)
[ILMV]EXXLFXR,
(SEQ ID NO: 81)
[ITV]P[ST]WR,
(SEQ ID NO: 103)
[LM]X[HR]LNX[ILM],
(SEQ ID NO: 131)
[NST]P[FM]XXX[ST]XK,
(SEQ ID NO: 199)
[RK]S[ML]AS,
(SEQ ID NO: 132)
[ST]PMXXAXXK,
(SEQ ID NO: 149)
AXX[MS]RKP,
(SEQ ID NO: 147)
CXEX[ILM]LF,
(SEQ ID NO: 142)
CXLXWSXC,
(SEQ ID NO: 36)
CXXFIAXC,
(SEQ ID NO: 20)
CXXXPCMXK,
(SEQ ID NO: 152)
CXXYWX[AC]XXK,
(SEQ ID NO: 108)
D[LM]DXFXK,
(SEQ ID NO: 11)
D[PA]X[ML]X[STA]XK,
(SEQ ID NO: 110)
DX[AC][GM][AFS]K,
(SEQ ID NO: 111)
DXAGXK,
(SEQ ID NO: 29)
DXSGIXXX[DGESN],
(SEQ ID NO: 85)
ETXS[LM]G,
(SEQ ID NO: 146)
FXE[HR]XK[PR],
(SEQ ID NO: 118)
FXQVCXK,
(SEQ ID NO: 117)
FXXV[MT]RK,
(SEQ ID NO: 8)
FXXVC[ENA]K,
(SEQ ID NO: 9)
HGWTK,
(SEQ ID NO: 99)
HXQCPXXXK,
(SEQ ID NO: 140)
K[DE]X[IV]PXXD,
(SEQ ID NO: 203)
K[DS]G[ILV]XW,
(SEQ ID NO: 33)
K[FY][LM]XFXQ,
(SEQ ID NO: 72)
K[WY]X[AP]FXQ,
(SEQ ID NO: 93)
KEXLXXWF,
(SEQ ID NO: 141)
KMXPWXQ,
(SEQ ID NO: 86)
KTPP[ILM],
(SEQ ID NO: 34)
KWXXFQQ,
(SEQ ID NO: 150)
KXG[ACT]PXXXXP,
(SEQ ID NO: 155)
KXP[FW]CP,
(SEQ ID NO: 21)
KXQTX[ST]M,
(SEQ ID NO: 91)
KXSXIEX[ILM],
(SEQ ID NO: 114)
KXX[IV]Q[FY]XD,
(SEQ ID NO: 39)
KXXGPXXLY,
(SEQ ID NO: 32)
LPFQQ,
(SEQ ID NO: 123)
M[ST][ACT]AK,
(SEQ ID NO: 124)
MT[AT][AS][AK],
(SEQ ID NO: 27)
P[EDT]WRXY,
(SEQ ID NO: 148)
PXWXXG[LM]D,
(SEQ ID NO: 40)
QGPT[LI],
(SEQ ID NO: 98)
QGPTXP,
(SEQ ID NO: 22)
QX[AS]MXPK,
(SEQ ID NO: 137)
QX[NSJFKXX[FL],
(SEQ ID NO: 17)
QX[PT]C[LM]XK,
(SEQ ID NO: 18)
QXP[MQ]LXK,
(SEQ ID NO: 19)
QXXC[LM]XKXXF,
(SEQ ID NO: 145)
R[FH]PEK,
(SEQ ID NO: 78)
RXPX[ST]LE,
(SEQ ID NO: 89)
RXX[CIJEXX[IL]F
(SEQ ID NO: 5)
RXXQE[DN]XXF,
(SEQ ID NO: 88)
RXXQEXXLF,
(SEQ ID NO: 135)
SAXY[AMS]XK,
(SEQ ID NO: 130)
STXXL[ST]XK,
(SEQ ID NO: 90)
SXXEDXLF,
(SEQ ID NO: 144)
WF[DE]XID,
(SEQ ID NO: 121)
WXX[IL]M[AL]K,
(SEQ ID NO: 154)
YCXAXKXC,
(SEQ ID NO: 151)
YWXYFXK,
(SEQ ID NO: 143)
YX[IR][FY]ML.

Also provide for herein is a peptide expression library, wherein the library comprises a library of nucleic acid sequences encoding a library of peptides, wherein the library of peptides comprises at least one distinct peptide, wherein the at least one distinct peptide comprises a binding motif, wherein the binding motif is selected from a motif group consisting of:

(SEQ ID NO: 79)
PXWR[IV]Y,
(SEQ ID NO: 27)
P[EDT]WRXY,
(SEQ ID NO: 81)
[ITV]P[ST]WR,
(SEQ ID NO: 84)
[EFY]TX[PS]LG,
(SEQ ID NO: 85)
ETXS[LM]G,
(SEQ ID NO: 86)
KTPP[ILM
(SEQ ID NO: 95)
D[LMY]SGI,
(SEQ ID NO: 96)
[ACP]XXXDXSGI,
(SEQ ID NO: 203)
K[DS]G[ILV]XW,
(SEQ ID NO: 97)
[AP]Q[EG]XTXXK,
(SEQ ID NO: 98)
QGPTXP,
(SEQ ID NO: 99)
HXQCPXXXK,
(SEQ ID NO: 18)
QXP[MQ]LXK,
(SEQ ID NO: 101)
LX[IL]LXXLN,
(SEQ ID NO: 102)
[FY]X[LV]LXXLN,
(SEQ ID NO: 202)
[GS][AS]AXXX[KR]K,
(SEQ ID NO: 104)
FP[AP][MT]XXK,
(SEQ ID NO: 105)
[FY]PXTXPK,
(SEQ ID NO: 21)
KXQTX[ST]M,
(SEQ ID NO: 107)
[ILM]LPXXXLD,
(SEQ ID NO: 113)
[FL][DN]XY[FY]XD,
(SEQ ID NO: 114)
KXX[IV]Q[FY]XD,
(SEQ ID NO: 117)
FXXV[MT]RK,
(SEQ ID NO: 118)
FXQVCXK,
(SEQ ID NO: 8)
FXXVC[ENA]K,
(SEQ ID NO: 14)
EGXLXMXK,
(SEQ ID NO: 119)
[DE]GXLX[LM]XK,
(SEQ ID NO: 120)
WWXXL[LMT]XK,
(SEQ ID NO: 121)
WXX[IL]M[AL]K,
(SEQ ID NO: 122)
[DE]PX[LM]X[ACS]XK,
(SEQ ID NO: 123)
M[ST][ACTJAK,
(SEQ ID NO: 124)
MT[AT][AS][AK],
(SEQ ID NO: 10)
[ED]XX[LM][TS][AC]XK,
(SEQ ID NO: 11)
D[PA]X[ML]X[STA]XK,
(SEQ ID NO: 129)
TPMXXX[ST]XK,
(SEQ ID NO: 130)
STXXL[ST]XK,
(SEQ ID NO: 131)
[NST]P[FM]XXX[ST]XK,
(SEQ ID NO: 132)
[ST]PMXXAXXK,
(SEQ ID NO: 134)
[AS]AX[FY]MXK,
(SEQ ID NO: 135)
SAXY[AMS]XK,
(SEQ ID NO: 136)
QXSFKXX[FMW],
(SEQ ID NO: 137)
QX[NS]FKXX[FL]
(SEQ ID NO: 140)
K[DE]X[IV]PXXD,
(SEQ ID NO: 141)
KMXPWXQ,
(SEQ ID NO: 142)
CXLXWSXC,
(SEQ ID NO: 143)
YX[IR][FY]ML,
(SEQ ID NO: 144)
WF[DE]XID,
(SEQ ID NO: 145)
R[FH]PEK,
(SEQ ID NO: 146)
FXE[HR]XK[PR],
(SEQ ID NO: 148)
PXWXXG[LM]D,
(SEQ ID NO: 149)
AXX[MS]RKP,
(SEQ ID NO: 36)
CXXFIAXC,
(SEQ ID NO: 9)
HGWTK,
(SEQ ID NO: 150)
KXG[ACT]PXXXXP,
(SEQ ID NO: 151)
YWXYFXK,
(SEQ ID NO: 152)
CXXYWX[AC]XXK,
(SEQ ID NO: 153)
CXVXPXMXK,
(SEQ ID NO: 20)
CXXXPCMXK,
(SEQ ID NO: 154)
YCXAXKXC,
(SEQ ID NO: 39)
KXXGPXXLY.

Also provide for herein is a peptide expression library, wherein the library comprises a library of nucleic acid sequences encoding a library of peptides, wherein the library of peptides comprises at least one distinct peptide, wherein the at least one distinct peptide comprises a binding motif, wherein the binding motif is selected from a motif group consisting of:

(SEQ ID NO: 134)
[AS]AX[FY]MXK,
(SEQ ID NO: 122)
[DE]PX[LM]X[ACS]XK,
(SEQ ID NO: 9)
HGWTK,
(SEQ ID NO: 145)
R[FH]PEK,
(SEQ ID NO: 97)
[AP]Q[EG]XTXXK,
(SEQ ID NO: 113)
[FL][DN]XY[FY]XD,
(SEQ ID NO: 149)
AXX[MS]RKP,
(SEQ ID NO: 108)
D[LM]DXFXK,
(SEQ ID NO: 14)
EGXLXMXK,
(SEQ ID NO: 21)
KXQTX[ST]M,
(SEQ ID NO: 39)
KXXGPXXLY,
(SEQ ID NO: 101)
LX[IL]LXXLN,
(SEQ ID NO: 195)
IPGS[FY],
(SEQ ID NO: 197)
A[FIY]SG[GHS].

Also provide for herein is a peptide library composition comprising: a) a library of nucleic acid sequences encoding a library of peptides, wherein the library of peptides comprises at least one distinct peptide, wherein the at least one distinct peptide comprises a binding motif, wherein the binding motif is selected from a motif group consisting of: RXX[CI]EXX[LI]F (SEQ ID NO:4), RXXQE[DN]XXF (SEQ ID NO:5), [MLIV]EXXLFXR (SEQ ID NO:6), D[ML]DXFXK (SEQ ID NO:7), FXXVC[ENA]K (SEQ ID NO:8), HGWTK (SEQ ID NO:9), [ED]XX[LM][TS][AC]XK (SEQ ID NO:10), D[PA]X[ML]X[STA]XK (SEQ ID NO:11), MT[TA][AS][KA] (SEQ ID NO:12), M[TS][ACT]AK (SEQ ID NO:13), EGXLXMXK (SEQ ID NO:14), FXXELXX[WY]F (SEQ ID NO:15), [FY]XX[QE]LXXWF (SEQ ID NO:16), QX[PT]C[LM]XK (SEQ ID NO:17), QXP[MQ]LXK (SEQ ID NO: 18), QXXC[LM]XKXXF (SEQ ID NO: 19), CXXXPCMXK (SEQ ID NO:20), KXQTX[ST]M (SEQ ID NO:21), QX[AS]MXPK (SEQ ID NO:22), [TS]PMXXAXXK (SEQ ID NO:23), STXXL[TS]XK (SEQ ID NO:24), QX[SN]FKXX[FL] (SEQ ID NO:25), RXPX[TS]L[ED] (SEQ ID NO:26), P[EDT]WRXY (SEQ ID NO:27), PXWR[VI]Y (SEQ ID NO:28), DXSGIXXX[DGESN] (SEQ ID NO:29), [APC]XXXDXSGI (SEQ ID NO:30), KTPP[LMI] (SEQ ID NO:31), LPFQQ (SEQ ID NO:32), K[FY][LM]XFXQ (SEQ ID NO:33), KWXXFQQ (SEQ ID NO:34), [CA]K[FY]XXFXQ (SEQ ID NO:35), CXXFIAXC (SEQ ID NO:36), CXXYWX[CA]XXK (SEQ ID NO:37), [LMI]LPXXXLD (SEQ ID NO:38), KXXGPXXLY (SEQ ID NO:39), QGPT[LI] (SEQ ID NO:40), KXG[TCA]PXXXXP (SEQ ID NO:41), WX[RK]A[DV]S (SEQ ID NO:42), [WTHN]XXXRA[VA]S (SEQ ID NO:43), [FY][RK]A[DAV][TS] (SEQ ID NO:44), CXXC[KR]AXS (SEQ ID NO:45), LX[AP]R[RKA]D (SEQ ID NO:46), FX[RK][RK]DT (SEQ ID NO:47), [RK][RKA]D[CTS]N (SEQ ID NO:48), [YF][RK]R[DA]XXX[PVR] (SEQ ID NO:49), WX[RK]MAD (SEQ ID NO:50), RMADXXR (SEQ ID NO:51), PQXXXDK[TSR] (SEQ ID NO:52), [IV]XXX[QR][DN]KXH (SEQ ID NO:53), D[STR]NG[TS] (SEQ ID NO:54), D[SKH]NGT (SEQ ID NO:55), [VI]DXXG[TS][IL](SEQ ID NO:56), DXXG[TS][FWY][MV] (SEQ ID NO:57), [AI]DXXGTX[CHT] (SEQ ID NO:58), CXXDXXG[ST]C (SEQ ID NO:59), DXNX[TS]XK[YF] (SEQ ID NO:60), PNG[TS]XXP (SEQ ID NO:61), GXVDWD (SEQ ID NO:62), CXPXCXEXXW (SEQ ID NO:63), [AT]RA[VL]X[NTE] (SEQ ID NO:64); and b) one or more display surfaces, wherein the at least one distinct peptide is capable of being expressed, and wherein each of the one or more display surfaces is configured to display one or more copies of at least one of the at least one distinct peptide.

In some aspects, the composition further comprises one or more display surfaces, wherein the at least one distinct peptide is capable of being expressed, and wherein each of the one or more display surfaces is configured to display one or more copies of at least one of the at least one distinct peptides.

In some aspects, the library of peptides comprises at least 2 distinct peptides each comprising a distinct binding motif, wherein each distinct binding motif is selected from the motif group. In some aspects, the library of peptides comprises at least 3, at least 4, or at least 5 distinct peptides each comprising a distinct binding motif, wherein each distinct binding motif is selected from the motif group. In some aspects, the library of peptides comprises at least 6, at least 7, at least 8, or at least 9 distinct peptides each comprising a distinct binding motif, wherein each distinct binding motif is selected from the motif group. In some aspects, the library of peptides comprises at least 10, at least 15, at least 20, at least 25, or at least 30 distinct peptides each comprising a distinct binding motif, wherein each distinct binding motif is selected from the motif group.

In some aspects, the at least one distinct peptide is capable of binding to an antibody associated with a SARS-CoV-2 infection.

In some aspects, the one or more display surfaces comprises a single display surface. In some aspects, the single display surface comprises a solid support. In some aspects, each of the at least one distinct peptides is attached at a defined location on the single display surface. In some aspects, the single display surface is a peptide microarray. In some aspects, the defined location on the peptide microarray is a defined grid location on the peptide microarray. In some aspects, the single display surface is a multi-well plate. In some aspects, the defined location in the multi-well plate is a defined well in the multi-well plate. In some aspects, the single display surface comprises a lateral flow assay. In some aspects, the defined location in the lateral flow assay is a defined location on a capillary membrane.

In some aspects, the one or more display surfaces comprises a plurality of display surfaces. In some aspects, the plurality of display surfaces is a plurality of biological entity surfaces. In some aspects, the biological entity comprises a cell, a virus, or a bacteriophage. In some aspects, the at least one distinct peptide is encoded by one or more distinct nucleic acid sequences capable of expressing the at least one distinct peptide in the plurality of biological entities. In some aspects, the at least one distinct peptide comprises a surface display peptide sequence configured to be expressed as part of the peptide and capable of directing the peptides for display on the biological entity surface. In some aspects, the biological entity is an Escherichia coli cell. In some aspects, the at least one distinct peptide comprises at least a portion of an Escherichia coli eCPX scaffold. In some aspects, the plurality of display surfaces is a plurality of microparticle surfaces. In some aspects, each of the plurality of display surfaces comprises a single species of the at least one distinct peptide.

In some aspects, the library of peptides is a random peptide library. In some aspects, the library of peptides is a non-random peptide library. In some aspects, the non-random library is biased to represent epitopes correlated with one or more diseases or conditions, and wherein at least one of the one or more diseases or conditions is a SARS-CoV-2 infection. In some aspects, the biased representation is at least greater than a representation in a random peptide library. In some aspects, the greater representation is statistically significant, wherein the statistical significance has a p-value less than 0.05. In some aspects, the non-random library is biased to represent epitopes through additional analysis of the epitopes and/or motifs that relate to SARS-CoV-2, such as Protein-based Immunome Wide Association Studies (PIWAS)-based analysis.

In some aspects, each of the peptides in the library of peptides is at least 5, 6, 7, 8, 9, 10, 11, 12, 13, 14, 15, 16, 17, 18, 19, or 20 amino acids in length. In some aspects, each of the peptides in the library of peptides is an identical defined length. In some aspects, the identical defined length is 5, 6, 7, 8, 9, 10, 11, 12, 13, 14, 15, 16, 17, 18, 19, or 20 amino acids in length. In some aspects, the identical defined length is 12 amino acids in length.

In some aspects, the library of peptides is selected from the group consisting of: a bacterial expression library, a yeast expression library, a bacteriophage expression library, a ribosomal expression library, and a mammalian expression library.

In some aspects, the one or more display surfaces or the distinct peptides is bound or capable of being bound directly or indirectly to a capture entity. In some aspects, the capture entity comprises a solid support selected from the group consisting of: an agarose bead, a sepharose bead, a magnetic bead, and a resin. In some aspects, the capture entity comprises a binding moiety that binds directly to the one or more display surfaces or the distinct peptides. In some aspects, the capture entity comprises a binding moiety that binds a binding molecule that binds to the one or more display surfaces or at least one of the distinct peptides. In some aspects, the binding molecule that binds to at least one of the distinct peptides is an antibody from a biological sample. In some aspects, the binding moiety is selected from the group consisting of: Protein A, Protein G, Protein A/G, and an antibody or antigen binding fragment thereof. In some aspects, the antibody or antigen binding fragment thereof comprises an anti-immunoglobulin antibody. In some aspects, the binding moiety comprises streptavidin and the one or more display surfaces, the distinct peptide, or the binding molecule that binds to the one or more display surfaces or at least one of the distinct peptides further comprises biotin. In some aspects, the capture entity further comprises a fluorophore.

In some aspects, displaying comprises presentation of each peptide for direct binding by a binding molecule. In some aspects, the binding molecule is selected from the group consisting of: an antibody or antigen-binding fragment thereof, an aptamer, a peptoid, and an affibody. In some aspects, the binding molecule is an antibody or antigen-binding fragment thereof.

In some aspects, each of the nucleic acid sequences encoding the library of peptides and each of the control binding targets is operably linked to at least one promoter. In some aspects, the at least one promoter is a constitutive promoter. In some aspects, the at least one promoter is an inducible promoter.

In some aspects, the nucleic acid sequences encoding the library of peptides further comprise polymerase chain reaction (PCR) primer target sequences configured for amplification of the nucleic acid sequences encoding the library of peptides.

A library composition comprising two or more of any of the peptide expression library compositions provided herein. In some aspects, each of the two or more of the peptide expression library compositions is in a separate container. In some aspects, the container is selected from the group consisting of: a well in a multi-well plate, a microcentrifuge tube, a test tube, a tube, and a PCR tube. In some aspects, the two or more of the peptide expression library compositions is 2, 3, 4, 5, 6, 7, 8, 9, 10-15, 16-24, 24-48, 48-96, or 96-384 peptide expression library compositions. In some aspects, the two or more of the peptide expression library compositions is at least 10, at least 20, at least 50, at least 100, at least 200, at least 300, at least 500, at least 1000, or at least 2000 expression library compositions.

Also provide for herein is a kit comprising the composition of any of the above claims and instructions for use.

Also provide for herein is a method of manufacturing any of the compositions or kits of the above claims.

Also provide for herein is a method of determining the presence of specimen antibodies specific for SARS-CoV-2 in a biological sample comprising: contacting any of the peptide expression libraries provided herein with a biological sample, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the at least one distinct peptide and the specimen antibodies; and determining specimen antibodies specific for SARS-CoV-2 are present in the biological sample when binding between at least one of the at least one distinct peptide and the specimen antibodies is detected.

Also provide for herein is a method of treating a subject known or suspected of suffering from a SARS-CoV-2 infection comprising assessing or having assessed whether the subject suffers from a SARS-CoV-2 infection, wherein the assessment comprises: contacting any of the peptide expression libraries provided herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the at least one distinct peptide and the specimen antibodies; and assessing the subject as: i) positive for the SARS-CoV-2 infection, or ii) negative for the SARS-CoV-2 infection; and administering or having administered a therapy to treat the SARS-CoV-2 infection when the subject when the subject is assessed as positive.

Also provide for herein is a method of determining whether a subject is a candidate for treatment of a SARS-CoV-2 infection comprising assessing or having assessed whether the subject suffers from a SARS-CoV-2 infection, wherein the assessment comprises: contacting any of the peptide expression libraries provided herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the at least one distinct peptide and the specimen antibodies; and diagnosing the subject as: i) positive for the SARS-CoV-2 infection, or ii) negative for the SARS-CoV-2 infection, determining the subject is a candidate for a treatment of the SARS-CoV-2 infection when the subject is assessed as positive.

Also provide for herein is a method of diagnosing a SARS-CoV-2 infection in a subject comprising: contacting any of the peptide expression libraries provided herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the at least one distinct peptide and the specimen antibodies; and diagnosing the subject as: i) positive for the SARS-CoV-2 infection, or ii) negative for the SARS-CoV-2.

In some aspects, the assessing or diagnosing comprises assessing or diagnosing the subject as: i) positive for the SARS-CoV-2 infection when binding between at least one of the at least one distinct peptides and the specimen antibodies is detected, or ii) negative for the SARS-CoV-2 infection when binding between less than at least one of the at least one distinct peptides and the specimen antibodies is detected. the assessing or diagnosing comprises the assessing or diagnosing comprises assessing or diagnosing the subject as: i) positive for the SARS-CoV-2 infection when binding between at least 2, at least 3, at least 4, or at least 5 distinct peptides of the at least one distinct peptides and the specimen antibodies is detected, or ii) negative for the SARS-CoV-2 infection when binding between less than at least 2, at least 3, at least 4, or at least 5 distinct peptides, respectively, of the at least one distinct peptides and the specimen antibodies is detected. In some aspects, the assessing or diagnosing comprises assessing or diagnosing the subject as: i) positive for the SARS-CoV-2 infection when binding between at least 6, at least 7, at least 8, at least 9, or at least 10 distinct peptides of the at least one distinct peptides and the specimen antibodies is detected, or ii) negative for the SARS-CoV-2 infection when binding between less than at least 6, at least 7, at least 8, at least 9, or at least 10 distinct peptides, respectively, of the at least one distinct peptides and the specimen antibodies is detected. In some aspects, the measuring comprises calculating a z-score for binding between the at least one distinct peptide and the specimen antibodies and the assessing or diagnosing comprises assessing or diagnosing the subject as: i) positive for the SARS-CoV-2 infection when the z-score meets or exceeds a threshold value, or ii) negative for the SARS-CoV-2 infection when the z-score is less than the threshold value.

In some aspects, the method further comprises assessing severity of the SARS-CoV-2 infection in the subject. In some aspects, assessing severity of the SARS-CoV-2 infection comprises: measuring the binding between the specimen antibodies and one or more distinct peptides associated with severe and/or moderate Covid disease; and diagnosing or assessing the infection as mild, moderate, or severe disease.

Also provided for herein is a method of assessing disease severity of a SARS-CoV-2 infection in a subject comprising: contacting any one of the peptide expression libraries described herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope; measuring the binding between the specimen antibodies and one or more distinct peptides associated with severe and/or moderate Covid disease; and diagnosing or assessing the infection as mild, moderate, or severe disease.

In some aspects, the method further comprises determining amino acid sequences of peptides in the library composition bound by the specimen antibodies. In some aspects, determining the amino acid sequences comprises isolating the specimen binding molecules bound to the one or more display surfaces and sequencing the nucleic acids encoding the distinct peptide to determine the amino acid sequences bound by the specimen binding molecules. In some aspects, the method further comprises determining binding motifs of peptides in the library composition bound by the specimen antibodies using a clustering algorithm. In some aspects, determining the amino acid sequences comprises isolating the specimen binding molecules bound to the one or more display surfaces and sequencing the nucleic acids encoding the distinct peptide to determine the amino acid sequences bound by the specimen binding molecules: i) determining a binding location of the specimen binding molecule on the one or more display surfaces; ii) correlating the binding location with the defined location of the distinct peptides to determine the amino acid sequences bound by the specimen binding molecules.

In some aspects, the sample is selected from the group consisting of: serum, blood, saliva, urine, tissue, tissue homogenates, stool, spinal fluid, and lysate derived from animal sources. In some aspects, the sample is human serum. In some aspects, the human serum is from a subject suspected of having a SARS-CoV-2 infection. In some aspects, the subject is suspected of having an acute SARS-CoV-2 infection. In some aspects, the subject is suspected of having a convalescent SARS-CoV-2 infection.

In some aspects, the binding molecules in the sample are antibodies. In some aspects, the antibodies comprise one or more antibodies known or suspected to recognize antigens or epitopes correlating a SARS-CoV-2 infection.

In some aspects, the contacting comprises mixing the sample, the peptide expression library composition, and the control binding molecule in a container selected from the group consisting of: a well in a multi-well plate, a microcentrifuge tube, a test tube, a tube, and a PCR tube.

In some aspects, the isolating step is selected from the group consisting of: magnetic isolation, bead centrifugation, resin centrifugation, and flow cytometry. In some aspects, the isolating step comprises contacting the sample with a capture entity. In some aspects, the capture entity is selected from the group consisting of: an agarose bead, a sepharose bead, a magnetic bead, and a resin. In some aspects, the capture entity comprises a binding moiety. In some aspects, the binding moiety is selected from the group consisting of: Protein A, Protein G, Protein A/G, and an antibody or antigen binding fragment thereof. In some aspects, the antibody or antigen binding fragment thereof comprises an anti-immunoglobulin antibody. In some aspects, the binding moiety comprises streptavidin and the specimen binding molecules further comprises biotin.

In some aspects, when the one or more display surfaces is a biological entity surface, the determining step further comprises the steps of: 1) purifying or having purified polynucleotides from the biological entity, wherein the polynucleotides comprise the nucleic acid sequences encoding the peptide bound by the isolated specimen binding molecules; 2) amplifying or having amplified the nucleic acid sequences encoding the peptide bound by the isolated specimen binding molecules; and 3) sequencing or having sequenced the amplified polynucleotides. In some aspects, the amplifying step further comprises adding a sample identifying nucleic acid sequence to a terminus of the amplified polynucleotides, wherein the sample identifying nucleic acid sequence is unique to the sample. In some aspects, the method further comprises the step of determining the identity of the sample identifying nucleic acid sequences.

In some aspects, the method has a sensitivity of at least 45%, at least 50%, at least 60%, at least 70%, at least 80%, at least 90%, or at least 95%. In some aspects, the method has a sensitivity of at least 52%, at least 53%, or at least 56%. In some aspects, the method has a sensitivity of at least 56%, at least 67%, or at least 76%. In some aspects, the method has a sensitivity of at least 50%, at least 73%, at least 75%, at least 88%, or at least 91%. In some aspects, the method has a sensitivity of at least 53%. In some aspects, the method has a sensitivity of at least 73%. In some aspects, the method has a sensitivity of at least 77%. In some aspects, the method has a sensitivity of at least 82%. In some aspects, the method has a sensitivity of at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, or at least 89%.

In some aspects, the method has a specificity of at least 90%, at least 91%, at least 92%, at least 93%, at least 94%, at least 95%, at least 96%, at least 97%, at least 98%, or at least 99%. In some aspects, the method has a specificity of 99%. In some aspects, the method has a sensitivity of at least 53% and a specificity of at least 99%. In some aspects, the method has a sensitivity of at least 73% and a specificity of at least 99%. In some aspects, the method has a sensitivity of at least 77% and a specificity of at least 99%. In some aspects, the method has a sensitivity of at least 82% and a specificity of at least 99%.

In some aspects, calculating the z-score comprises calculating a composite z-score for binding between each of the at least one distinct peptides specific for SARS-CoV-2 and the specimen antibodies.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A presents enrichment for the IgG motif RXX[CI]EXX[LI]F (SEQ ID NO:4) in SARS-CoV-2 (n=157) relative to controls (n=500).

FIG. 1B presents enrichment for the IgG motif [MLIV]EXXLFXR (SEQ ID NO:6) in SARS-CoV-2 (n=157) relative to controls (n=500).

FIG. 1C presents enrichment for the IgG motif D[ML]DXFXK (SEQ ID NO:7) in SARS-CoV-2 (n=157) relative to controls (n=500).

FIG. 1D presents enrichment for the IgG motif FXXVC[ENA]K (SEQ ID NO:8) in SARS-CoV-2 (n=157) relative to controls (n=500).

FIG. 1E presents enrichment for the IgG motif HGWTK (SEQ ID NO:9) in SARS-CoV-2 (n=157) relative to controls (n=500).

FIG. 1F presents enrichment for the IgG motif [ED]XX[LM][TS]I[AC]XK (SEQ ID NO:10) in SARS-CoV-2 (n=157) relative to controls (n=500).

FIG. 1G presents enrichment for the IgG motif QXP[MQ]LXK (SEQ ID NO:18) in SARS-CoV-2 (n=157) relative to controls (n=500).

FIG. 1H presents enrichment for the IgG motif EGXLXMXK (SEQ ID NO:14) in SARS-CoV-2 (n=157) relative to controls (n=500).

FIG. 1I presents enrichment for the IgG motif CXXXPCMXK (SEQ ID NO:20) in SARS-CoV-2 (n=157) relative to controls (n=500).

FIG. 1J presents enrichment for the IgG motif FXXELXX[WY]F (SEQ ID NO: 15) in SARS-CoV-2 (n=157) relative to controls (n=500).

FIG. 2A presents enrichment for the IgM motif [WTHN]XXXRA[VA]S (SEQ ID NO:43) in SARS-CoV-2 (n=155) relative to controls (n=499).

FIG. 2B presents enrichment for the IgM motif WX[RK]A[DV]S (SEQ ID NO:42) in SARS-CoV-2 (n=155) relative to controls (n=499).

FIG. 2C presents enrichment for the IgM motif WX[RK]MAD (SEQ ID NO:50) in SARS-CoV-2 (n=155) relative to controls (n=499).

FIG. 2D presents enrichment for the IgM motif D[STR]NG[TS] (SEQ ID NO:54) in SARS-CoV-2 (n=155) relative to controls (n=499).

FIG. 2E presents enrichment for the IgM motif D[SKH]NGT (SEQ ID NO:55) in SARS-CoV-2 (n=155) relative to controls (n=499).

FIG. 2F presents enrichment for the IgM motif [VI]DXXG[TS][IL] (SEQ ID NO:56) in SARS-CoV-2 (n=155) relative to controls (n=499).

FIG. 2G presents enrichment for the IgM motif [RK][RKA]D[CTS]N (SEQ ID NO:48) in SARS-CoV-2 (n=155) relative to controls (n=499).

FIG. 2H presents enrichment for the IgM motif PQXXXDK[TSR] (SEQ ID NO:52) in SARS-CoV-2 (n=155) relative to controls (n=499).

FIG. 2I presents enrichment for the IgM motif GXVDWD (SEQ ID NO:62) in SARS-CoV-2 (n=155) relative to controls (n=499).

FIG. 2J presents enrichment for the IgM motif CXXDXXG[ST]C (SEQ ID NO:59) in SARS-CoV-2 (n=155) relative to controls (n=499).

FIG. 3A presents IgG diagnostic epitope motif panel composite z-scores for CoV-2 cohorts and pre-pandemic controls. Shown are the percentage of samples for each cohort exceeding the established composite z-score cutoff of 25.

FIG. 3B presents IgM diagnostic epitope motif panel composite z-scores for CoV-2 cohorts and pre-pandemic controls. Shown are the percentage of samples for each cohort exceeding the established composite z-score cutoff of 25.

FIG. 4A presents ROC curves analysis used in determining the IgG diagnostic motif panel at a fixed specificity of 99%.

FIG. 4B presents ROC curves analysis used in determining the IgM diagnostic motif panel at a fixed specificity of 99%.

FIG. 5 presents IgG disease severity epitope motif panel composite z-scores for CoV-2 cohorts and pre-pandemic controls. Shown are the percentage of samples for each cohort exceeding the established composite z-score cutoff of 40.

FIG. 6A presents performance of the LASSO-determined severity motif panel demonstrating predictive groupings of mild vs. moderate/severe in training (left panel) and testing (right panel) curated Covid sample groups.

FIG. 6B presents ROC curve performance of the LASSO-determined severity motif panel.

### DETAILED DESCRIPTION

### 1.1. Definitions

Terms used in the claims and specification are defined as set forth below unless otherwise specified.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Numerical values provided herein can sometimes be considered to be modified by the term about, where context makes clear that the ranges encompassed by the modification are consistent with operability of the invention and definiteness of the claims.

The term “motif” as used herein comprises an amino acid sequence pattern, which comprises preferred amino acids at each position of a peptide sequence. For example, RXX[CI]EXX[LI]F (SEQ ID NO:4) where “X” is any amino acid and each letter corresponds to the conventional one-letter amino acid code. The notation [XYZ] within a motif means that the indicated position comprises one amino acid that is selected from “X or Y or Z”. Motifs may alternatively be presented graphically as a sequence “logo,” wherein the frequencies of occurrence of individual amino acids at each position in a motif are represented by the height of the character (e.g. one letter amino acid code) at that position. A larger letter indicates a higher frequency of occurrence.

The term “epitope” refers to the part of an antigen molecule/s to which a binding molecule (e.g., an antibody) attaches itself. For example, in the case of a protein antigen, the epitope can be the amino acid sequence or protein structural region to which the binding molecule (e.g., antibody) binds, in other words the amino acid sequence or protein structural region responsible for mediating a non-covalent interaction between a binding molecule and its cognate target.

The term “epitope repertoire” as used herein comprises the set of all antigens recognized, or bound by, by a binding molecules (e.g., antibodies) within a sample, or group of samples. For example, the epitope repertoire may refer to the set of all peptides or antigens recognized, or bound by, by binding molecules (e.g., antibodies) within a sample, or group of samples.

The term “enrichment” or “enriched” as used herein refers to the number of observations of a peptide, pattern, or motif within an epitope repertoire divided by the number expected within a random dataset of equivalent size. For example, in a hypothetical 9-mer peptide library (-XXXXXXXXX-), where X is any amino acid, the pattern QPXXPFX[ED](SEQ ID NO:3) is expected to occur once in every 800,000 ((laa/20aa)4x(2aa/20aa)x2) random sequences (aa=amino acid). If 4 million sequences were determined, then one would expect to observe five (5) occurrences (i.e., once in every 800,000 sequences). As an example, if the pattern was actually observed in 50 unique peptides sequences (i.e. 50 observations) in an epitope repertoire, then the pattern would be “enriched” by 10-fold versus random.

The term “threshold” as used herein refers to the magnitude or intensity that must be exceeded for a certain reaction, phenomenon, result, or condition to occur or be considered relevant. For example, the threshold can be a numerical value above which enrichment is considered relevant. The relevance can depend on context, e.g., it may refer to a positive, reactive or statistically significant relevance.

As used here, the terms “massively parallel signature sequencing” (MPSS) or “next generation sequencing” (NGS) and the like are used interchangeably to refer to high throughput nucleic acid sequencing (HTS) approaches. Platforms for NGS that rely on different sequencing technologies are commercially available from a number of vendors such as Pacific Biosciences, Ion Torrent from Thermo Fisher, 454 Life Sciences, Illumina, Inc. (e.g., MiSeq, NextSeq, HiSeq) and Oxford Nanopore. For a review of NGS technologies, see, e.g., van Dijk E L et al. Ten years of next-generation sequencing technology. Trends Genet. 2014 September; 30(9):418-26, herein incorporated by reference in its entirety for all purposes.

The term “clustering algorithm” as used herein refers to a computational algorithm used to perform “cluster analysis.” Cluster analysis or clustering is the task of grouping a set of objects in such a way that objects in the same group (called a cluster) are more similar (in some sense or another) to each other than to those in other groups (clusters). A variety of clustering algorithms are known to those of skill in the art. See, e.g., Bailey et al. (Fitting a mixture model by expectation maximization to discover motifs in biopolymers. Proc Int Conf Intell Syst Mol Biol, 1994. 2: p. 28-36); Amstutz et al. (In vitro display technologies: novel developments and applications. Curr Opin Biotechnol, 2001. 12(4): p. 400-5); and Gould-Rothberg et al. (Massively parallel (“next-generation”) DNA sequencing. Clin Chem, 2015. 61(7): p. 997-8.), each herein incorporated by reference in their entirety for all purposes.

The term “disease” refers to an abnormal condition affecting the body of an organism. The term “disorder” refers to a functional abnormality or disturbance. The terms disease or disorder are used interchangeably herein unless otherwise noted or clear given the context in which the term is used. The terms disease and disorder may also be referred to collectively as a “condition.”

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al.).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect.

The term “therapeutically effective amount” is an amount that is effective to ameliorate a symptom of a disease. In some contexts, a therapeutically effective amount can be a “prophylactically effective amount” as prophylaxis can be considered therapy, provided such interpretation does not adversely impact any determination of the validity of any claim for any reason.

It must be noted that, as used in the specification and the appended claims, the singular forms “a,” “an,” and “the” include plural referents unless the context clearly dictates otherwise.

### 1.2. Peptide Display Systems

In one aspect, peptide display systems are provided. In a particular aspect, peptide display systems comprising peptides corresponding to epitopes associated with SARS-CoV-2 are provided. A peptide display system has at a minimum the peptides to be displayed, e.g., peptides corresponding to epitopes associated with SARS-CoV-2, and one or more display surfaces.

1.2.1. Peptides

A peptide display system can include one or more distinct peptides. The terms “peptide,” “polypeptide,” “amino acid sequence,” “peptide sequence,” and “protein” are used interchangeably to refer to two or more amino acids linked together and imply no particular length. As used herein, a “distinct peptide” refers to a peptide having a specific amino acid sequence. Amino acids can be naturally occurring or synthetic (e.g., non-natural amino acids or amino acid analogs). Peptides can be naturally occurring peptides, e.g., a peptide having an amino acid sequence known to occur entirely in an organism and without additional non-natural modifications. Peptides can be non-naturally occurring peptides, e.g., a peptide having (1) an amino acid sequence not known to occur in its entirety in an organism and/or (2) containing non-natural modifications, such as non-natural amino acids or amino acid analogs. Amino acids and peptides can also comprise, or be further modified to comprise, reactive groups, such as reactive groups for attaching amino acids or peptides to display surfaces (e.g., solid substrates), reactive groups for labeling amino acids or peptides, or reactive groups for attaching other moieties of interest to amino acids or peptides. Reactive groups include, but are not limited to, chemically-reactive groups such as reactive thiols (e.g., maleimide based reactive groups), reactive amines (e.g., N-hydroxysuccinimide based reactive groups), “click chemistry” groups (e.g., reactive alkyne groups), and aldehydes bearing formylglycine (FGly).

A peptide display system can have any number of distinct peptides useful for the present invention, such as any number of distinct peptides suitable for displaying on a display surface such that the distinct peptides are capable of recognition by a binding molecule. It is understood that references to numbers of distinct peptides does not refer to the absolute quantity (i.e., copies, occurrences, or concentration) of each distinct peptide, but instead to the number of distinct peptide species. In some examples, the number of distinct peptides possible is determined by the physical limitations of a display surface, such as the size of a peptide microarray or number of wells in a multi-well plate.

A peptide display system includes at least one distinct peptide. A peptide display system can include one or more distinct peptides. A peptide display system can include two or more distinct peptides. A peptide display system can include at least 3, at least 4, or at least 5 distinct peptides. A peptide display system can include at least 6, at least 7, at least 8, at least 9, or at least 10 distinct peptides. A peptide display system can include at least 3 distinct peptides. A peptide display system can include at least 4 distinct peptides. A peptide display system can include at least 5 distinct peptides. A peptide display system can include at least 6 distinct peptides. A peptide display system can include at least 7 distinct peptides. A peptide display system can include at least 8 distinct peptides. A peptide display system can include at least 9 distinct peptides. A peptide display system can include at least 10 distinct peptides. A peptide display system can include at least 15, at least 20, at least 25, or at least 30 distinct peptides. A peptide display system can include at least 40, at least 50, at least 60, at least 70, at least 80, at least 90, or at least 100 distinct peptides. A peptide display system can include at least 200, at least 300, at least 400, at least 500, at least 600, at least 700, at least 800, at least 900, or at least 1000 distinct peptides. A peptide display system can include at least 300,000 distinct peptides. A peptide display system can include 300,000 or fewer distinct peptides. A peptide display system can include 384 or fewer, 96 or fewer, 48 or fewer, 24 or fewer, or 12 or fewer distinct peptides.

Typically, peptides in a peptide display system and/or a library can also all fall within a range of lengths. For example, the peptides, or epitope portions thereof, in a peptide display system and/or a library may be different lengths, but all fall within a defined range of lengths. The selected range can be any length useful for the present invention, such as any length suitable for displaying an epitope sequence on a display surface such that it is capable of recognition by a binding molecule. The peptides in a peptide display system and/or a library can be at least 5, 6, 7, 8, 9, 10, 11, 12, 13, 14, 15, 16, 17, 18, 19, or 20 amino acids in length. The peptides in a peptide display system and/or a library can also be 5-30, 5-25, 5-20, 5-15, 5-10, 10-30, 10-25, 10-20, or 10-15 amino acids in length. The peptides in a peptide display system and/or a library can also be 7-14, 8-14, 9-14, 10-14, 11-14, 12-14, 7-13, 8-13, 9-13, 10-13, 11-13, 12-13, 7-12, 8-12, 9-12, 10-12, 11-12, 7-11, 8-11, 9-11, or amino acids in length. If desired, the peptides in a peptide display system and/or a library can also be greater than 30, greater than 40, greater than 50, greater than 75, greater than 100, greater than 200, or greater than 300 amino acids in length. The epitope portions of peptides in a peptide display system and/or a library can be at least 5, 6, 7, 8, 9, 10, 11, 12, 13, 14, 15, 16, 17, 18, 19, or 20 amino acids in length. The epitope portions of peptides in a peptide display system and/or a library can also be 5-30, 5-25, 5-20, 5-15, 5-10, 10-30, 10-25, 10-20, or 10-15 amino acids in length. The epitope portions of peptides in a peptide display system and/or a library can also be 7-14, 8-14, 9-14, 10-14, 11-14, 12-14, 7-13, 8-13, 9-13, 10-13, 11-13, 12-13, 7-12, 8-12, 9-12, 10-12, 11-12, 7-11, 8-11, 9-11, or amino acids in length. If desired, the epitope portions of peptides in a peptide display system and/or a library can also be greater than 30, greater than 40, greater than 50, greater than 75, greater than 100, greater than 200, or greater than 300 amino acids in length.

Peptides, or the epitope portions thereof, in a peptide display system and/or a library can also be an identical defined length, i.e., all the peptides in the a peptide display system and/or the library have the same number of amino acids. The defined length can be any length useful for the present invention, such as any length suitable for displaying an epitope sequence on a display surface such that it is capable of recognition by a binding molecule. The defined length can be 5, 6, 7, 8, 9, 10, 11, 12, 13, 14, 15, 16, 17, 18, 19, or 20 amino acids in length. In an illustrative non-limiting example, each distinct peptide in the peptide display system and/or the library is 12 amino acids in length. In another illustrative non-limiting example, each distinct peptide in the peptide display system and/or the library has a peptide portion that is 12 amino acids in length that is capable of recognition by a binding molecule (e.g., each distinct peptide has an epitope sequence that is 12 amino acids in length). The defined length (e.g., the defined length of 5, 6, 7, 8, 9, 10, 11, 12, 13, 14, 15, 16, 17, 18, 19, or 20 amino acids) can refer to only the epitope portion of the peptide, such as the epitope portion of a peptide in an E. coli surface display peptide library, where the full-length peptide also contains a cell surface display peptide (see Section 1.2.3). The defined length (e.g., the defined length of 5, 6, 7, 8, 9, 10, 11, 12, 13, 14, 15, 16, 17, 18, 19, or 20 amino acids) can refer to the length of the full-length peptide. The defined length (e.g., the defined length of 5, 6, 7, 8, 9, 10, 11, 12, 13, 14, 15, 16, 17, 18, 19, or 20 amino acids) can refer to the length of the full-length peptide, where the full-length peptide is the epitope portion of the peptide, such as in some instances the distinct peptides used in a peptide microarray.

A peptide can include the specific amino acids or amino acid classes (e.g., hydrophobic, hydrophilic, acidic, basic, or steric amino acid) as described by a peptide motif sequence. A peptide can include the amino acids or amino acid classes as described by a peptide motif sequence where the full peptide sequence is not known to occur in its entirety in an organism. A peptide can include the amino acids or amino acid classes as described by a peptide motif sequence where the motif sequence is derived from an organism, while the full peptide sequence is not known to occur in its entirety in an organism. A peptide can include the amino acids or amino acid classes as described by a peptide motif sequence where the full peptide sequence is derived from an organism. A peptide can include the specific amino acids or amino acid classes representative of a motif class. As used herein, a “motif class” refers to peptide motif sequences that can include a common specific amino acid or amino acid class in at least 3 specific positions. As an illustrative non-limiting example, peptide motif sequences RXX[CI]EXX[LI]F (SEQ ID NO:4) and RXXQE[DN]XXF (SEQ ID NO:5) can be considered as part of the same motif class given each share at least the same amino acid at positions 1, 5, and 9. As another illustrative non-limiting example, peptide motif sequences MT[TA][AS][KA](SEQ ID NO:12) and M[TS][ACT]AK (SEQ ID NO:13) can be considered as part of the same motif class given each share the option of the same amino acid at positions 1, 2, 3, 4, and 5. In general, a motif class represents peptide motif sequences that are known or are likely to encompass the same epitope derived from an organism.

Peptides, or the epitope portions thereof, in a peptide display system and/or a library can also be selected that are associated with different characteristics of a disease or have different applications of the peptide display system. For example, peptides can be selected that are those capable of assessing or diagnosing a subject as positive for a disease. In another example, peptides can be selected that are those capable of assessing disease status, such as severity of a disease. The peptide display system can include all selected peptides that are associated with different characteristics of a disease, such as both selected peptide capable of assessing or diagnosing a subject as positive for a disease and selected peptide capable of assessing disease status, such as severity of a disease. In other examples, peptide display systems separately include different selections of peptides that are associated with different characteristics of a disease, such as a first peptide display system including selected peptides capable of assessing or diagnosing a subject as positive for a disease and a second peptide display system including selected peptide capable of assessing disease status, such as severity of a disease. In other examples, peptide display systems separately include different selections of peptides that are selected through additional analysis of the epitopes and/or motifs that relate to SARS-CoV-2, such as Protein-based Immunome Wide Association Studies (PIWAS)-based analysis. PIWAS analysis is described in Haynes et al. (Protein-based Immunome Wide Association Studies (PIWAS) for the discovery of significant disease-associated antigens. bioRxiv 2020.03.18.997759 (2020)), herein incorporated by reference for all purposes.

1.2.2. Peptide Libraries

A peptide display system can include a library of peptides. As used herein, a “library of peptides” or a “peptide library” refers to a collection of a peptide fragments. Peptide libraries can be used for screening purposes. In general, a peptide library contains a large variety of distinct peptides. For example, the diversity of the library (i.e., the number of distinct peptides in a library and sometimes referred to as “complexity” of the library) can be more than 10⁴, more than 10⁵, more than 10⁶, more than 10⁷, more than 10⁸, more than 10⁹, more than 10¹⁰, or more than 10¹¹ distinct peptides.

The library can be a random peptide library where the amino acid sequences are unbiased. A particular embodiment of a random/unbiased library is one constructed to represent all possible amino acid sequences of designated length(s). Peptide libraries can be produced by a peptide expression library (see Section 1.3). Peptide libraries include, but are not limited to, peptide libraries produced using bacterial expression libraries, yeast expression libraries, bacteriophage expression libraries, ribosome expression libraries, and mammalian expression libraries. Particular peptide libraries and peptide expression libraries useful for the present invention are described in more detail in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference in their entirety for all purposes.

A peptide library can also be a non-random library where the amino acid sequences are biased in their representation. A peptide library can also be a non-random library where the amino acid sequence motifs are biased in their representation. For example, a library can be biased to represent, over represent, predominantly represent, or only represent amino acid sequences, or amino acid sequence motifs, characteristic of a particular feature, such as epitopes or antigens associated with a particular disease (e.g., a bacterial infection, a viral infection, a parasitic infection, an autoimmune disorder, cancer, allergies etc.), condition, species (e.g., mammal, human, bacteria, virus etc.), protein, class of proteins, protein motif (e.g., phosphorylation motifs, binding motifs, protein domains, etc.), amino acid property (e.g., hydrophobic, hydrophilic, acidic, basic, or steric amino acid properties), or any other subset of amino acid sequences that is rationally designed. In an illustrative non-limiting example, a library is biased to represent epitopes associated with SARS-CoV-2. In another illustrative non-limiting example, a library is biased to represent epitopes associated with a panel of diseases, such as a panel including SARS-CoV-2. A library can be biased to also avoid certain amino acid sequences or motifs. A biased library can be said to be enriched for one or more particular sequences, peptides, patterns, or motifs. Bias can be quantitated using statistical measurements, such as determining the representation of a distinct peptide or motif relative to its expected representation by chance in a random library. The bias of peptide representation in a library can be statistically significant, such as a distinct peptide or peptide motif having a greater representation than in a random library of the same size with a statistical significance having a p-value less than 0.05. In another example, the non-random library is biased to represent epitopes through additional analysis of the epitopes and/or motifs that relate to SARS-CoV-2, such as Protein-based Immunome Wide Association Studies (PIWAS)-based analysis. PIWAS analysis is described in Haynes et al. (Protein-based Immunome Wide Association Studies (PIWAS) for the discovery of significant disease-associated antigens. bioRxiv 2020.03.18.997759 (2020)), herein incorporated by reference for all purposes.

Each distinct peptide in the library of peptides can share a common feature, such as all representing epitopes associated with SARS-CoV-2. A library can also have one or more groups of peptides in the library of peptides, each group independently sharing a common feature, such as each group independently representing epitopes associated with particular disease (e.g., a peptide panel representing a variety of diseases).

A peptide library can also combine the features of a non-random and random peptide library. For example, one or more select positions within an amino acid sequence may be a constant amino acid and other positions within the sequence may be fully random or biased based on other properties. In other examples, one or more select positions within an amino acid sequence may be selected from a defined subset of amino acids. One skilled in the art will appreciate that the various biases described can combined to achieve a desired purpose of the peptide library, such as a targeted screen.

1.2.3. Display Surfaces

A peptide display system has one or more display surfaces. As used herein, a “display surface” refers to any surface that can be configured to display (i.e., present) peptides in a manner suitable for recognition by their respective binding molecules, e.g., antibodies, such as antibodies present in a biological sample.

Display surfaces can be a biological entity surface (e.g., the outer membrane surface of a cell). Biological entities that can be used include, but are not limited to, a mammalian cell, a yeast, a bacteria, a virus, and a bacteriophage. The members of a library of peptides (e.g., candidate peptides) and/or selected peptide sequences (e.g., epitope motifs, such as epitopes associated with SARS-CoV-2) can be engineered to be expressed on the surface of a cell, such as constructing the library of nucleic acid sequences encoding the library of peptides or the nucleic acid sequences encoding the selected peptide sequences to also encode a cell surface display peptide sequence configured to be expressed as part of the peptide and capable of directing the peptides for display on the biological entity surface. Illustrative non-limiting examples of E. coli cell surface display systems and display surfaces are described in greater detail in in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference in their entirety for all purposes.

Display surfaces can include solid supports. As described herein, a “solid support” refers to a material to which other entities can be attached (i.e., immobilized) and typically refers to materials that are non-biological in nature (e.g., not the surface of an organism, such as a cell membrane, viral surface, or bacteriophage surface). Materials that can be used to produce solid supports include, but are not limited to, glass, functionalized glass, silicon, germanium, gallium arsenide, gallium phosphide, silicon dioxide, sodium oxide, silicon nitrade, nitrocellulose, nylon, polytetrafluoroethylene, polyvinylidendiflouride, polystyrene, polycarbonate, methacrylates, or combinations thereof. The solid support and/or the surface of the solid support can be homogeneous or heterogeneous, e.g., composed of a homogeneous or heterogeneous material.

In some embodiments, the display system uses single display surface. In some embodiments, the single surface is a solid support. Solid supports with a single surface can include, but are not limited to, a planar surface, e.g., a multi-well plate, peptide microarray (also sometimes referred to as a peptide chip), or lateral flow assay (also sometimes referred to as a lateral flow immunochromatographic assay or lateral low test). A planar surface can be flat, essentially flat, concave, or convex. Peptides can be attached to the single surface, e.g., a multi-well plate, peptide microarray, or lateral flow assay capillary membrane, such that each distinct peptide species is attached to the surface at a defined location (e.g., in a particular well, a particular grid location of a microarray, or a particular region of a capillary membrane) such that the defined location can be used as a reference for identification of the peptide species. Attachment of multiple distinct peptide species at defined locations can be said to be “arrayed” on the display surface or to form a “peptide array.”

In some embodiments, the display system uses a plurality of display surfaces. In some embodiments, the plurality of display surfaces are a plurality of solid supports. A plurality of solid supports can include, but are not limited to, a plurality of beads (also sometimes referred to as microbeads, microparticles, nanoparticles, or nanobeads). Examples of beads include, but are not limited to, agarose beads, polystyrene beads, magnetic beads (e.g., Dynabeads™ThermoFisher or Pierce), polymers (e.g., dextran), synthetic polymers (e.g., Sepharose™), or any other bead suitable for displaying peptides. In some embodiments, the plurality of display surfaces are biological entity surfaces, such as a plurality of cells, viruses, bacteriophage, or ribosomes.

In some embodiments, each display surface (or defined location of an array surface) is configured to display one or more copies of a single species of a peptide (i.e., only distinct peptides having the same amino acid sequence). In a non-limiting illustrative example, a display surface is a multi-well plate well that contains multiple copies of a single species of a peptide, or display surface is a defined location of a peptide array grid that contains multiple copies of a single species of a peptide. In another non-limiting illustrative example, a composition has a plurality of beads or biological entities where each bead or biological entity displays multiples copies a single species of a peptide. In a further example, a display library can have a plurality of display surfaces, where each display surface is configured to display one or more copies of a single species of a peptide (i.e., a distinct peptide), and where multiple peptide species are represented by individual members of the plurality having a single distinct peptide species.

In some embodiments, each display surface is configured to display one or more copies of multiple distinct peptide species. In a non-limiting illustrative example, a composition has a single peptide microarray display surface where the peptide microarray displays multiples copies of multiple peptide species on the same surface.

In some embodiments, peptides are attached (also sometimes referred to as “immobilized”) to the display surfaces. Peptides can be attached either their N or C terminus. In some embodiments, proteins/peptides, nucleic acids, or both attached to the display surface and can be modified for use in the present invention. The display surfaces (e.g., the solid supports) and/or the peptides can be modified or functionalized to allow attachment of the peptide to the display surface. Methods of attaching proteins/peptides and nucleic acids are known to those skilled in the art and include, but are not limited to, use of chemically reactive groups such as reactive thiols (e.g., maleimide based reactive groups), reactive amines (e.g., N-hydroxysuccinimide based reactive groups), “click chemistry” groups (e.g., reactive alkyne groups), aldehydes bearing formylglycine (FGly) and other cognate modifications (e.g., biotin-streptavidin pairs, disulfide linkages, polyhistidine-nickel). Examples of modifications include chemical modifications that allow formation of covalent bonds with proteins (e.g., activated aldehyde groups) and modifications that specifically pair a modified display surface with a peptide having a cognate modification (e.g., biotin-streptavidin pairs, disulfide linkages, polyhistidine-nickel, or “click-chemistry” modifications such as azido-alkynyl pairs). Modifications can also include functional groups, such as an imide functional group, an amine functional group, a hydroxyl functional group, a carboxyl functional group, an aldehyde functional group, and/or a sulfhydryl functional group. Methods of attaching peptides to microarrays are known to those skilled in the art, for example, as described in more detail in Macbeath, G.; Schreiber, S. L. Science, 2000, 289, 1760-1763; MacBeath, G.; et al. J. Am. Chem. Soc., 1999, 121, 7967-7968; Falsey, J. R.; et al. Bioconj. Chem., 2001, 12, 346-353; Houseman, B. T.; et al. Nat. Biotechnol., 2002, 20, 270-274; Lesaicherre, M. L.; et al. Bioorg. Med. Chem. Lett. 2002, 12, 2079-2083; Lesaicherre, M. L.; et al. Bioorg. Med. Chem. Lett. 2002, 12, 2085-2088; Uttamchandani, M.; et al. Bioorg. Med. Chem. Lett., 2003, 13, 2997-3000; Oliver, C. et al. Bioconj. Chem., 2003, 14, 430-439; Newman, J. R. S.; Keating, A. E. Science, 2003, 300, 2097-2101; Kiyonaka, S.; et al. Nat. Materials, 2004, 3, 58-64; Li, S.; et al. Am. Chem. Soc., 2004, 126, 4088-4089; Cheng, C. W.; et al. Bioorg. Med. Chem. Lett., 2004, 14, 1987-1990; Houseman, B. T.; et al. Langmuir., 2004, 19, 1522-1531; Lesaicherre, M. L.; Lue et al. J. Am. Chem. Soc. 2002, 124, 8768-8769; Lue, R. Y. P.; et al. J. Am. Chem. Soc., 2004, 126, 1055-1062; Salisbury, C. M. et al. J. Am. Chem. Soc. 2002, 124, 14868-14870; Zhu, Q.; et al. Org. Lett., 2003, 5, 1257-1260; and Soellner, M. B.; et al. J. Am. Chem. Soc., 2003, 125, 11790-11791, each herein incorporated by reference in their entirety for all purposes. Non-limiting illustrative examples include in situ peptide arrays synthesis techniques where unique peptide sequences are simultaneously synthesized on a glass surfaces, or spotting (also sometimes referred to as printing) techniques where presynthesized peptides are spotted onto a suitably derivatized display surface, e.g., a glass surface. Both in situ peptide arrays synthesis techniques and spotting techniques are described in more detail in U.S. Pub. Nos. US20110071043A1 and US20140087963A1, each herein incorporated by reference in their entirety for all purposes. Lateral flow assays are based on ELISA techniques and generally involve attaching and/or spotting antibodies or analytes to a capillary membrane configured such that applying a liquid sample on one end of the capillary membrane will “flow” the samples along the membrane to interact with the analyte and/or antibody. Non-limiting exemplary sandwich-type lateral flow assays are described in greater detail in U.S. Pat. Nos. 4,168,146 and 4,366,241, each of which is herein incorporated by reference in their entirety for all purposes. Other exemplary device configurations and/or assay formats are also described in greater detail in U.S. Pat. Nos. 5,395,754; 5,670,381; and 6,194,220, each of which is herein incorporated by reference in their entirety for all purposes.

The display surfaces (e.g., the solid supports) can also possess further modifications or functionalized properties that aid in the methods and processes of capturing and isolating binding molecules and/or other applications, such as sequencing (e.g., barcodes facilitating NGS applications). As non-limiting illustrations, solid supports can have magnetic properties that allow for magnetic isolation or have fluorescent molecules attached, for example, fluorophores that facilitate fluorescence-activated cell sorting (FACS). Other properties may also include the ability to remove (e.g., elute or cleave off) bound peptides from the display surface, such as reversible linkers (e.g., disulfide reduction of thiol linkers) or protease cleavage sites (e.g., TEV protease recognition motif).

### 1.3. Peptide Expression Libraries

In one aspect, peptide expression library compositions are provided. In a particular aspect, peptide expression library compositions comprising peptides corresponding to epitopes associated with SARS-CoV-2 are provided, as described in further detail below.

Peptide expression library compositions comprise a library of nucleic acid sequences encoding a library of peptides. As used herein, a “library of peptides” or a “peptide library” refers to a collection of a peptide fragments typically used for screening purposes. The terms “peptide,” “polypeptide,” “amino acid sequence,” “peptide sequence,” and “protein” are used interchangeably to refer to two or more amino acids linked together and imply no particular length. Amino acids and peptides can be naturally occurring or synthetic (e.g., non-natural amino acids or amino acid analogs). Amino acids and peptides can also comprise, or be further modified to comprise, reactive groups, such as reactive groups for attaching amino acids or peptides to solid substrates, reactive groups for labeling amino acids or peptides, or reactive groups for attaching other moieties of interest to amino acids or peptides. Reactive groups include, but are not limited to, chemically-reactive groups such as reactive thiols (e.g., maleimide based reactive groups), reactive amines (e.g., N-hydroxysuccinimide based reactive groups), “click chemistry” groups (e.g., reactive alkyne groups), and aldehydes bearing formylglycine (FGly).

In general, a peptide expression library contains a large variety of distinct peptides. For example, the diversity of the library (sometimes referred to as “complexity” of the library) can be at least 10⁴, at least 10⁵, at least 10⁶, at least 10⁷, at least 10⁸, at least 10⁹, at least 10¹⁰, or at least 10¹¹ distinct peptides. The library can be a random peptide expression library where the amino acid sequences are unbiased. A particular embodiment of a random/unbiased library is one constructed to represent all possible amino acid sequences of designated length(s).

A peptide expression library can also be a non-random library where the amino acid sequences are biased in their representation. For example, a library can be biased to represent, over represent, predominantly represent, or only represent amino acid sequences characteristic of a particular feature, such as epitopes or antigens associated with a particular disease (e.g., a bacterial infection, a viral infection, a parasitic infection, an autoimmune disorder, cancer, allergies etc.), condition, species (e.g., mammal, human, bacteria, virus etc.), protein, class of proteins, protein motif (e.g., phosphorylation motifs, binding motifs, protein domains, etc.), amino acid property (e.g., hydrophobic, hydrophilic, acidic, basic, or steric amino acid properties), or any other subset of amino acid sequences that is rationally designed. A library can be biased to also avoid certain amino acid sequences or motifs. A library can be biased to also avoid certain amino acid sequences or motifs. A library can be biased to represent one distinct peptide species of a particular motif, e.g., one distinct peptide species representative of an epitope or epitope motif. For example, a library can have one distinct peptide species representative of peptide motif sequences RXX[CI]EXX[LI]F (SEQ ID NO:4) and RXXQE[DN]XXF (SEQ ID NO:5). A library can be biased to represent one distinct peptide species of a particular motif class, e.g., one distinct peptide species representative of motifs that are known or are likely to encompass the same epitope. For example, a library can have one distinct peptide species representative of either peptide motif sequence RXX[CI]EXX[LI]F (SEQ ID NO:4) or peptide motif sequence RXXQE[DN]XXF (SEQ ID NO:5), which can be considered as part of the same motif class. Without wishing to be bound by theory, inclusion of more than one peptide featuring slight variations, e.g., more than one peptide from the same motif class, can yield better enrichments and/or capture slightly different subsets of subjects.

A peptide library can also combine the features of a non-random and random peptide library. For example, one or more select positions within an amino acid sequence may be a constant amino acid and other positions within the sequence may be fully random or biased based on other properties. In other examples, one or more select positions within an amino acid sequence may be selected from a defined subset of amino acids. One skilled in the art will appreciate that the various biases described can combined to achieve a desired purpose of the peptide library, such as a targeted screen.

Typically, peptides in a library can also all fall within a range of lengths. For example, the peptides in a library may be different lengths, but all fall within a defined range of lengths. The selected range can be any length useful for the present invention, such as any length suitable for displaying an epitope sequence capable of recognition by a binding molecule. The peptides in a library can be at least 5, 6, 7, 8, 9, 10, 11, 12, 13, 14, 15, 16, 17, 18, 19, or 20 amino acids in length. The peptides in a library can also be 5-30, 5-25, 5-20, 5-15, 5-10, 10-30, 10-25, 10-20, or 10-15 amino acids in length. The peptides in a library can also be 7-14, 8-14, 9-14, 10-14, 11-14, 12-14, 7-13, 8-13, 9-13, 10-13, 11-13, 12-13, 7-12, 8-12, 9-12, 10-12, 11-12, 7-11, 8-11, 9-11, or amino acids in length. If desired, the peptides in the library can also be greater than 30, greater than 40, greater than 50, greater than 75, greater than 100, greater than 200, or greater than 300 amino acids in length.

Peptides in a library can also be an identical defined length, i.e., all the peptides in the library have the same number of amino acids. The defined length can be any length useful for the present invention, such as any length suitable for displaying an epitope sequence capable of recognition by a binding molecule. The defined length can be 5, 6, 7, 8, 9, 10, 11, 12, 13, 14, 15, 16, 17, 18, 19, or 20 amino acids in length.

The nucleic acid sequences in the peptide expression library can be constructed to achieve a desired library property including those described above, such as peptide diversity, peptide randomization or biasing, and/or peptide length. For example, nucleic acid sequences in the peptide expression library can be biased for expression in specific organisms, such as codon optimization for expression in bacterial, viral, plant, yeast/fungal, insect, mammalian, or human expression systems. Any suitable nucleic acid allowing expression of the peptides of interest may be used. In general, the nucleic acid will be a vector. As used herein, a “vector” refers to nucleic acid construct capable of directing the expression of a gene of interest, typically in a host organism, such as a bacterial cell, a mammalian cell (e.g., a human cell), a bacteriophage, a yeast cell, an insect cell, or a plant cell. A vector typically contains the appropriate transcriptional and translational regulatory nucleotide sequences recognized by the desired host for peptide expression, such as promoter sequences. A promoter sequence can be a constitutive promoter. A promoter sequence can be an inducible promoter, where transcription of the encoded sequences is induced by addition of an analyte, chemical, or other molecule, such as a Tet-on system. A variation of an inducible promoter system is a system where transcription is actively repressed, and addition of an analyte, chemical, or other molecule removes the repression, such as addition of arabinose for an arabinose operon promoter or a Tet-off system. A vector can also include elements that facilitate vector construction and production, such as restriction sites, sequences that direct vector replication, drug selection genes or other selectable markers, and any other elements useful for cloning and library production. A typical vector can be a double stranded DNA plasmid in which the nucleic acid sequences encoding the desired peptides is inserted using standard cloning techniques in a location and orientation capable of directing peptide expression. Other vectors include, but are not limited to, nucleic acid constructs useful for in vitro transcription and translation, linear nucleic acid constructs, and single-stranded DNA or RNA nucleic acid constructs.

In general, the number of copies of a specific nucleic acid sequence for each of the candidate peptides is present at a roughly equivalent number, though some variation in number may occur due to probability. A typical peptide expression library can contain more than one copy of a specific nucleic acid sequence (e.g., multiple copies of the same vector). However, in examples where a plurality of samples each contain members of the peptide expression library, the absolute number of each of the candidate peptides may not be equivalent between samples. For example, zero or one copy of a specific nucleic acid sequence can be present in a given sample while one or more copies may be present in another given sample. While the number of copies of a specific nucleic acid sequence need not be identical to the number of copies of other specific nucleic acid sequences, it is generally assumed that about the same number of sequences are present for each of the candidate peptides.

Peptide expression libraries include, but are not limited to, bacterial expression libraries, yeast expression libraries, bacteriophage expression libraries, ribosome expression libraries, and mammalian expression libraries. Particular peptide libraries and peptide expression libraries useful for the present invention are described in more detail in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference in their entirety for all purposes.

### 1.3.1. Arrayed Peptide Expression Libraries

In a series of embodiments, a composition is composed of two or more of the peptide expression library compositions described above. The two or more peptide expression library compositions can each be contained in a separate container, such as a well in a multi-well plate, a microcentrifuge tube, a test tube, a tube, and a PCR tube. Each of the separate containers can comprise the same library of nucleic acid sequences encoding the library of peptides or a collection of different peptide expression libraries. The collection of peptide expression library compositions can be 2, 3, 4, 5, 6, 7, 8, 9, 10-15, 16-24, 24-48, 48-96, or 96-384 peptide expression library compositions. The collection of peptide expression library compositions can be at least 10, at least 20, at least 50, at least 100, at least 200, at least 300, at least 500, at least 1000, or at least 2000 expression library compositions.

### 1.4. Binding Molecules

As used herein, “binding molecules” refer to those molecules that specifically and selectively bind a binding target(s). In a typical non-limiting example, a binding molecule is an antibody. An antibody can be a monoclonal antibody, which are typically produced from cultured antibody-producing cell lines, or a polyclonal antibody, which are typically produced by collecting the antibody containing serum of an animal immunized with the antigen or epitope of interest, or fragment thereof. Antibodies can also refer to antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In other examples, the binding molecule(s) can refer to binding molecules other than antibodies including, but not limited to, aptamers, peptoids, and affibodies.

A binding molecule (e.g., an antibody) or antigen binding portion thereof is said to “recognize” the epitope (or more generally, the antigen) to which the binding molecule specifically binds, and the epitope (or more generally, the antigen) is said to be the “recognition specificity” or “binding specificity” of the binding molecule.

A binding molecule is said to bind to its specific antigen or epitope with a particular affinity. As described herein, “affinity” refers to the strength of interaction of non-covalent intermolecular forces between one molecule and another. The affinity, i.e., the strength of the interaction, can be expressed as a dissociation equilibrium constant (KD), wherein a lower KD value refers to a stronger interaction between molecules. “Specific binding,” as used herein, refers to an affinity between an ABS and its cognate antigen or epitope in which the KD value is below 10⁻⁶M, 10⁻⁷M, 10⁻⁸M, 10⁻⁹M, or 10⁻¹⁰M. KD values of binding molecules (e.g. antibodies) are measured by methods well known in the art including, but not limited to, bio-layer interferometry (e.g., Octet/FORTEBIO®), surface plasmon resonance (SPR) technology (e.g., Biacore®), and cell binding assays.

### 1.5. Biological Samples

As used herein, a “biological sample” refers to any material known to contain or suspected to contain specimen binding molecules (e.g., antibodies). In general, the sample will be a liquid. The sample can be a material that originated as a liquid or can be material processed to be in liquid form. The sample can be the material directly isolated from a source (i.e., untreated) or it can be further processed for use in the method (e.g., diluted, filtered, cell depleted, particulate depleted, assayed, preserved, or other otherwise pre-processed). Samples include, but are not limited to, serum, blood, saliva, urine, tissue, tissue homogenates, stool, spinal fluid, and lysate derived from animal sources. The sample can include a mixture of different source materials. A sample can be a bodily fluid isolated from any animal that produces or suspected to produce the binding molecule of interest. The animal can be known or suspected of having a disease. The animal can also be known or suspected of having binding molecules that bind antigens or epitopes associated with the disease. In an illustrative non-limiting example, the sample can be processed serum from a human suspected to have a specific disease and suspected to produce antibodies that bind epitopes that correlate with the disease. Diseases include, but are not limited to, a bacterial infection, a viral infection, a parasitic infection, an autoimmune disorder, cancer, and an allergy.

### 1.6. Capture Entities

In one example of the invention, the display surfaces, binding molecules, and/or peptides described above are bound to or capable of being bound to a capture entity. As used herein, a “capture entity” refers to any entity useful for capturing or isolating a display surface, a binding molecule, and/or a peptide. The binding molecule can be any of the binding molecules described herein, such as a binding molecule in a biological sample (e.g., serum antibodies in a patient's sample). The display surface can be any of the display surfaces described herein, such as a display surface in a peptide display system (e.g., a solid support or biological entity).

In a typical example, the capture entity comprises a solid support. Solid supports are described in greater detail in Section 1.2.3 titled “Display surfaces.”

The capture entities (e.g., the solid supports) can also be modified or functionalized to bind and capture (i.e., immobilize) display surfaces, binding molecules, and/or peptides. Such modifications can be attaching to or coating the surface of a capture entity with a “binding moiety” that binds the display surfaces, binding molecules, and/or peptides. Examples include binding moieties that specifically bind antibodies, such as Protein A, Protein G, Protein A/G, Protein L, or an anti-immunoglobulin antibody. Other examples include chemical modifications that allow formation of covalent bonds with proteins (e.g., activated aldehyde groups) and modifications that facilitate specific pairing via cognate modifications (e.g., biotin-streptavidin pairs, disulfide linkages, polyhistidine-nickel, or “click-chemistry” modifications such as azido-alkynyl pairs). As a non-limiting illustration, a binding molecule can be modified to contain a biotin moiety (i.e., biotinylated) and a capture entity can be modified to contain streptavidin on its surface. Capture entities can also possess further modifications or functionalized properties that aid in the methods and processes of capturing and isolating display surfaces, binding molecules, and/or peptides. As non-limiting illustrations, capture entities can have magnetic properties that allow for magnetic isolation, or capture entities can have fluorescent molecules attached that allow, for example, fluorescence-activated cell sorting (FACS). Other properties include the ability to remove (e.g., elute or cleave off) captured display surfaces, binding molecules (and/or their respective binding target), and/or peptides if desired.

In a non-limiting example, in the methods describe herein where a binding molecule is used to bind a binding target, the binding molecule can either be immobilized on a capture entity prior to the binding molecule binding its target (e.g., prior to contacting or mixing the peptide library with the binding molecule), or the binding molecule can be immobilized on a capture entity subsequent to the binding molecule binding its target. Similarly, display surfaces and/or peptides can either be immobilized on a capture entity prior to contacting another reagent, such as a sample, or subsequent to contacting another reagent.

As a non-limiting illustration of a capture entity, a magnetic bead is functionalized with Protein A/G (e.g., Pierce). The Protein A/G bead is subsequently used to capture antibodies bound to their respective epitope targets.

### 1.7. Uses, Methods, and Assays Associated with SARS-CoV-2 Epitope Motifs

In particular aspects, any of the peptide display systems or peptide expression libraries described herein can be used in an assay. Examples of types of assays the peptide display systems or peptide expression libraries described herein are useful for include, but are not limited to, binding assays (e.g., antibody binding assays), diagnostic assays, vaccine efficacy and safety studies, immune status monitoring assays. In particular, provided for herein are assays, such as binding assays (e.g., antibody binding assays), diagnostic assays, vaccine efficacy and safety studies, immune status monitoring assays, using peptide display systems or peptide expression libraries containing epitopes associated with SARS-CoV-2. Accordingly, the peptide display systems or peptide expression libraries described herein can be formulated for use in an assay method, such as any of the assays described herein, including, but not limited to, a method of determining the presence of specimen binding molecules (e.g., antibodies) specific for SARS-CoV-2, a method of diagnosing a subject for SARS-CoV-2, a method of treating a subject known or suspected of suffering from SARS-CoV-2, a method of determining whether a subject is a candidate for treatment of SARS-CoV-2, and/or a method of determining the binding affinity of specimen binding molecules (e.g., antibodies) for an epitope associated with SARS-CoV-2. The peptide display systems or peptide expression libraries described herein can also be used to assess severity of disease. For example, motifs associated with severe and/or moderate disease can be used to classify a subject as having a severe and/or moderate disease based on motif analysis, or determine a course of treatment for a subject classified as having a severe and/or moderate disease based on motif analysis. Different peptide display systems or peptide expression libraries can be used in combination. Different motif panels can also be used in combination. For example, a first peptide display system or motif panel can be used to diagnose a subject and a second peptide display system or motif panel can be used to assess disease severity.

In one aspect, a method of determining the presence of specimen binding molecules (e.g., antibodies) specific for SARS-CoV-2 in a biological sample is provided. The method of determining the presence of specimen binding molecules (e.g., antibodies) specific for SARS-CoV-2 in a biological sample includes: contacting any of the display systems and/or peptide expression libraries described herein with a biological sample (e.g., a biological sample from a subject), wherein the biological sample contains a plurality of binding molecules (e.g., antibodies), wherein the plurality of binding molecules is known or suspected to contain specimen binding molecules specific for SARS-CoV-2, and wherein the contacting is under conditions sufficient for the specimen binding molecules specific for SARS-CoV-2 to bind a cognate epitope; measuring the binding between the distinct peptides of the display systems and/or peptide expression libraries and the specimen antibodies; and determining specimen binding molecules specific for SARS-CoV-2 are present in the biological sample when binding between at least one of the distinct peptides and the specimen binding molecules is detected. The assay can determine the presence of binding molecules (e.g., antibodies) that bind multiple distinct peptides. The assay can determine the presence of binding molecules (e.g., antibodies) that bind at least one of the multiple distinct peptides. The assay can determine the presence of binding molecules (e.g., antibodies) that bind at least 2, 3, 4, 5, 6, 7, 8, 9, 10 or more distinct peptides of the multiple distinct peptides. The assay can determine the presence of binding molecules (e.g., antibodies) that bind at least one of the at least one distinct peptides. The assay can determine the presence of binding molecules (e.g., antibodies) that bind the at least two distinct peptides. The assay can determine the presence of binding molecules (e.g., antibodies) that bind at least one of the at least two distinct peptides. The assay can determine the presence of binding molecules (e.g., antibodies) that bind 3, 4, 5, 6, 7, 8, 9, 10 or more distinct peptides. The assay can determine the presence of binding molecules (e.g., antibodies) that bind at least one of the 3, 4, 5, 6, 7, 8, 9, 10 or more distinct peptides. The assay can determine the presence of binding molecules (e.g., antibodies) that bind each of the one or more distinct peptides on the array. The assay can determine the presence of binding molecules (e.g., antibodies) that bind a motif encompassing the one or more distinct peptides on the array. The assay can determine the presence of binding molecules (e.g., antibodies) that bind a motif class encompassing the one or more distinct peptides on the array. The binding molecules that bind the multiple distinct peptides can be multiple distinct binding molecules that bind the multiple distinct peptides. The binding molecules that bind the multiple distinct peptides can include a single distinct binding molecule that binds two or more of the multiple distinct peptides, i.e., include a cross-reactive binding molecule. Determining the presence can also include determining the quantity or relative quantity (e.g., concentration) of a distinct binding molecule for a distinct peptide. Determining the presence can also include determining the quantity or relative quantity (e.g., concentration) of binding molecules for a peptide motif.

In one aspect, a method of diagnosing SARS-CoV-2 in a subject is provided. The method of diagnosing SARS-CoV-2 in a subject includes: contacting any of the display systems and/or peptide expression libraries described herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen binding molecules specific for SARS-CoV-2 to bind a cognate epitope; measuring the binding between the distinct peptides of the display systems and/or peptide expression libraries and the specimen antibodies; and diagnosing the subject. The subject can be diagnosed as: i) positive for the SARS-CoV-2 when binding between at least one of the at least one distinct peptides and the specimen antibodies is detected, or ii) negative for the SARS-CoV-2 when binding between less than the at least one of the at least one distinct peptides and the specimen antibodies is detected. The subject can be diagnosed as: i) positive for the SARS-CoV-2 infection when binding between at least 2, at least 3, at least 4, or at least 5 distinct peptides of the at least one distinct peptides and the specimen antibodies is detected, or ii) negative for the SARS-CoV-2 infection when binding between less than at least 2, at least 3, at least 4, or at least 5 distinct peptides, respectively, of the at least one distinct peptides and the specimen antibodies is detected. The subject can be diagnosed as: i) positive for the SARS-CoV-2 infection when binding between at least 6, at least 7, at least 8, at least 9, or at least 10 distinct peptides of the at least one distinct peptides and the specimen antibodies is detected, or ii) negative for the SARS-CoV-2 infection when binding between less than at least 6, at least 7, at least 8, at least 9, or at least 10 distinct peptides, respectively, of the at least one distinct peptides and the specimen antibodies is detected. The subject can be diagnosed as: i) positive for the SARS-CoV-2 infection when a z-score meets or exceeds a threshold value, or ii) negative for the SARS-CoV-2 infection when a z-score is less than the threshold value. A z-score for each motif can be calculated as the enrichment value minus the mean enrichment for all samples divided by the standard deviation of all samples. A z-score can be a value calculated for a single motif A z-score can be a composite z-score value (also referred to herein as a summed z-score, diagnostic score, or composite diagnostic score) in which the z-scores for each motif are summed for a specific disease. Methods of diagnosing a subject for SARS-CoV-2 can include assessing disease severity. Methods of diagnosing a subject for SARS-CoV-2 can include determining whether binding between at least 2, at least 3, at least 4, or at least 5, at least 6, at least 7, at least 8, at least 9, or at least 10 distinct peptides (or motifs) binds to peptides associated with a specific disease status (e.g., moderate or severe Covid). Methods of diagnosing a subject for SARS-CoV-2 can include determining whether a z-score for exceeds a disease classifier threshold value, such as calculating z-scores for motifs associated with a specific disease status (e.g., moderate or severe Covid). Methods of assessing disease severity can include using the same display system and/or peptide expression library used in diagnosing or assessing a subject as positive or negative for the disease. Methods of assessing disease severity can include using a different display system and/or peptide expression library used in diagnosing or assessing a subject as positive or negative for the disease, e.g., a display system limited to a panel of motifs associated with a specific disease status (e.g., moderate or severe Covid).

In one aspect, a method of treating a subject known or suspected of suffering from SARS-CoV-2 is provided. The method of treating the subject includes: assessing or having assessed whether the subject suffers from SARS-CoV-2, wherein the assessment includes contacting any of the display systems and/or peptide expression libraries described herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen binding molecules specific for SARS-CoV-2 to bind a cognate epitope; measuring the binding between the distinct peptides of the display systems and/or peptide expression libraries and the specimen antibodies; assessing the subject; and administering the therapy when the subject is assessed as positive. The subject can be assessed as: i) positive for the SARS-CoV-2 when binding between at least one of the at least one distinct peptides and the specimen antibodies is detected, or ii) negative for the SARS-CoV-2 when binding between less than at least one of the at least one distinct peptides and the specimen antibodies is detected. The subject can be assessed as: i) positive for the SARS-CoV-2 infection when binding between at least 2, at least 3, at least 4, or at least 5 distinct peptides of the at least one distinct peptides and the specimen antibodies is detected, or ii) negative for the SARS-CoV-2 infection when binding between less than at least 2, at least 3, at least 4, or at least 5 distinct peptides, respectively, of the at least one distinct peptides and the specimen antibodies is detected. The subject can be assessed as: i) positive for the SARS-CoV-2 infection when binding between at least 6, at least 7, at least 8, at least 9, or at least 10 distinct peptides of the at least one distinct peptides and the specimen antibodies is detected, or ii) negative for the SARS-CoV-2 infection when binding between less than at least 6, at least 7, at least 8, at least 9, or at least 10 distinct peptides, respectively, of the at least one distinct peptides and the specimen antibodies is detected. The subject can be assess as: i) positive for the SARS-CoV-2 infection when a z-score meets or exceeds a threshold value, or ii) negative for the SARS-CoV-2 infection when a z-score is less than the threshold value. A z-score can be a value calculated for a single motif A z-score can be a composite z-score value. Therapies for treating SARS-CoV-2 can be any of method known to those skilled in the art. Methods of treating a subject known or suspected of suffering from SARS-CoV-2 can include assessing disease severity. Methods of treating a subject known or suspected of suffering from SARS-CoV-2 can include determining whether binding between at least 2, at least 3, at least 4, or at least 5, at least 6, at least 7, at least 8, at least 9, or at least 10 distinct peptides (or motifs) binds to peptides associated with a specific disease status (e.g., moderate or severe Covid). Methods of treating a subject known or suspected of suffering from SARS-CoV-2 can include determining whether a z-score for exceeds a disease classifier threshold value, such as calculating z-scores for motifs associated with a specific disease status (e.g., moderate or severe Covid). Methods of assessing disease severity can include using a different display system and/or peptide expression library used in diagnosing or assessing a subject as positive or negative for the disease, e.g., a display system limited to a panel of motifs associated with a specific disease status (e.g., moderate or severe Covid).

In one aspect, a method of determining whether a subject is a candidate for treatment of SARS-CoV-2 is provided. The method of treating the subject includes: assessing or having assessed whether the subject suffers from SARS-CoV-2, wherein the assessment includes contacting any of the display systems and/or peptide expression libraries described herein with a biological sample from the subject, wherein the biological sample comprises a plurality of antibodies, wherein the plurality of antibodies is known or suspected to comprise specimen antibodies specific for SARS-CoV-2, and wherein the contacting is under conditions sufficient for the specimen binding molecules specific for SARS-CoV-2 to bind a cognate epitope the distinct peptides of the display systems and/or peptide expression libraries and the specimen antibodies; assessing the subject; and determining the subject is a candidate for a treatment of SARS-CoV-2 when the subject is assessed as positive. The subject can be assessed as: i) positive for the SARS-CoV-2 when binding between at least one of the at least one distinct peptides and the specimen antibodies is detected, or ii) negative for the SARS-CoV-2 when binding between less than at least one of the at least one distinct peptides and the specimen antibodies is detected. The subject can be assessed as: i) positive for the SARS-CoV-2 infection when binding between at least 2, at least 3, at least 4, or at least 5 distinct peptides of the at least one distinct peptides and the specimen antibodies is detected, or ii) negative for the SARS-CoV-2 infection when binding between less than at least 2, at least 3, at least 4, or at least 5 distinct peptides, respectively, of the at least one distinct peptides and the specimen antibodies is detected. The subject can be assessed as: i) positive for the SARS-CoV-2 infection when binding between at least 6, at least 7, at least 8, at least 9, or at least 10 distinct peptides of the at least one distinct peptides and the specimen antibodies is detected, or ii) negative for the SARS-CoV-2 infection when binding between less than at least 6, at least 7, at least 8, at least 9, or at least 10 distinct peptides, respectively, of the at least one distinct peptides and the specimen antibodies is detected. The subject can be assessed as: i) positive for the SARS-CoV-2 infection when a z-score meets or exceeds a threshold value, or ii) negative for the SARS-CoV-2 infection when a z-score is less than the threshold value. A z-score can be a value calculated for a single motif A z-score can be a composite z-score value. Therapies for treating SARS-CoV-2 can be any of method known to those skilled in the art. Methods of determining whether a subject is a candidate for treatment can include assessing disease severity. Methods of determining whether a subject is a candidate for treatment of SARS-CoV-2 can include determining whether binding between at least 2, at least 3, at least 4, or at least 5, at least 6, at least 7, at least 8, at least 9, or at least 10 distinct peptides (or motifs) binds to peptides associated with a specific disease status (e.g., moderate or severe Covid). Methods of determining whether a subject is a candidate for treatment of SARS-CoV-2 can include determining whether a z-score for exceeds a disease classifier threshold value, such as calculating z-scores for motifs associated with a specific disease status (e.g., moderate or severe Covid). Methods of assessing disease severity can include using a different display system and/or peptide expression library used in diagnosing or assessing a subject as positive or negative for the disease, e.g., a display system limited to a panel of motifs associated with a specific disease status (e.g., moderate or severe Covid).

In one aspect, a method of determining the binding affinity of specimen binding molecules (e.g., antibodies) for an epitope associated with SARS-CoV-2 in a biological sample is provided. The method of determining the binding affinity includes: contacting the display system and/or peptide expression library with a biological sample from the subject, wherein the biological sample contains a plurality of binding molecules (e.g., antibodies), wherein the plurality of binding molecules is known or suspected to contain specimen binding molecules specific for SARS-CoV-2, and wherein the contacting is under conditions sufficient for the specimen binding molecules specific for SARS-CoV-2 to bind a cognate epitope; and measuring the binding affinity between the at least two distinct peptides and the specimen binding molecules. Measuring the binding affinity can include determining if a binding molecule specifically binds an epitope associated with SARS-CoV-2.

As used herein, “contacting” refers to any method of bringing the specimen binding molecules in proximity to and under conditions sufficient for binding to their respective binding targets, e.g., the distinct peptides in the display system and/or protein expression library. The contacting of the different components can be performed in any suitable order. Providing suitable conditions for a binding molecule (e.g., an antibody) to bind a respective binding target (e.g., epitope or epitope motif) can include controlling conditions, including but not limited to, temperature, pH, salt, buffer, binding molecule concentration, and/or target concentration.

Contacting can include mixing all the compositions together. Mixing can be performed in a container, such as a well in a multi-well plate, a microcentrifuge tube, a test tube, a tube, and a PCR tube. Mixing can include rotating, incubating, pipetting, inverting, vortexing, shaking, or otherwise mechanically disturbing components.

“Isolating” steps used herein can be any method useful for retrieving specimen and control binding molecules. Isolation can involve the use of capture entities, described in more detail in Section 1.6. Isolation methods include, but are not limited to magnetic isolation, bead centrifugation, resin centrifugation, and FACS. A particular isolation method can be selected based on the properties of a capture entity, if used, for example magnetic isolation of magnetic beads or FACS isolation of fluorescent beads.

“Measuring” steps, as used herein, in general can use any method for detecting binding between a binding molecule and its cognate epitope, such as optical scanning, an ELISA, fluorescent detection, digital serology, flow-cytometry, surface plasmon resonance, bio-layer interferometry, lateral flow assay, or combinations thereof. In a particular non-limiting example, the E. coli expression library described herein can be used in conjunction with digital serology to measure binding. The binding measurements can be quantitative, such as determining binding affinity. The binding measurements can be qualitative, such as determining a binary result of whether a binding molecule does or does not bind a distinct peptide (e.g., determining whether the measured binding exceeds a threshold for the binding molecule to be considered to bind or specifically bind a distinct peptide).

Nucleic acid sequence determination steps, as used herein, in general can use any method for sequencing and/or quantifying nucleic acid, such next generation sequencing (NGS) or quantitative polymerase chain reaction (qPCR). Examples of NGS technologies include massively parallel sequencing techniques and platforms, such as Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies. In some embodiments, the determining step contains the steps of 1) purifying the nucleotide from the biological entity; 2) amplifying the purified nucleic acid sequences encoding a peptide bound by the isolated specimen binding molecules; and 2) sequencing the amplified nucleotides. The nucleic acid to be sequenced can also be further modified or processed to facilitate sequencing. For example, nucleic acid can be modified for multiplexed high-throughput sequencing of multiple samples simultaneously, such as adding a sample identifying nucleic acid sequence unique to the sample to terminus of the amplified nucleotides during the amplification step.

Various nucleic acid sequences (e.g., sequences encoding a library of peptides) can be differentiated from each other during the sequence determining step(s). Differentiating various nucleic acid sequences includes differentiating portions of nucleic acid sequences, such as differentiating the different sequences in a vector (e.g., differentiating a nucleic acid sequence encoding a binding target from other vector nucleic acid sequences). Sequences can be differentiated based on specific characteristics, such as position within a sequence, identity of adjacent sequences, known identity of sequences, or combinations thereof. Sequence alignment algorithms, such as those known in the art, can be used to identify, quantify, and differentiate the different sequences.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder described herein. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment),

As used herein, the term “administering,” refers to the placement an agent as disclosed herein into a subject by a method or route which results in at least partial localization of the agents at a desired site.

Any of the peptides, such as any of the SARS-CoV-2 specific epitopes, described herein can be used for any purpose. For example, any of the SARS-CoV-2 specific epitopes can be used for therapeutic purposes known to those skilled in the art, such as formulation of SARS-CoV-2 specific epitopes, or proteins or peptides including the same, for use in a vaccine.

The epitopes, peptides, and/or motifs herein or obtained using the methods herein can be additionally analyzed by methods known in the art, such as by bioinformatic analysis. An illustrative example of bioinformatic analysis includes Protein-based Immunome Wide Association Studies (PIWAS)-based analysis. PIWAS analysis is described in Haynes et al. (Protein-based Immunome Wide Association Studies (PIWAS) for the discovery of significant disease-associated antigens. bioRxiv 2020.03.18.997759 (2020)), herein incorporated by reference for all purposes. Use of bioinformatic analysis, such as PIWAS analysis, can include analysis that informs any of the diagnostic and/or assessment methods described herein, such as selecting specific epitopes, peptides, and/or motifs for the diagnostic and/or assessment methods described herein.

1.7.1. Diagnostic Thresholds

Diagnostic thresholds, e.g., whether a sample is considered positive for a disease by the number of distinct peptides bound, a z-score, and/or a composite z-score, can be established that meet desired criteria of a person of skill in the art and in general balance sensitivity and specificity. The number and identity of specific amino acid sequences and/or motifs can be selected to achieve a desired sensitivity and specificity. In general, a single peptide or motif can be sufficient to achieve a desired sensitivity and/or specificity. For example, a single immunodominant epitope, or fragment and/or motif thereof, can be used alone to achieve a desired sensitivity and/or specificity. In other examples, binding to multiple peptides or motifs is used to achieve a desired sensitivity and/or specificity, such a determining binding to two or more distinct peptides and/or calculating a composite z-score.

A diagnostic threshold criterion can evaluate where a sample is considered positive for a disease, the severity of the disease, or both.

A diagnostic threshold criterion can include an assay achieving a desired specificity. Specificity can be established with reference to a control sample or set of samples, such as correctly identifying as negative a set of control samples known to be negative for the disease (e.g., negative for SARS-CoV-2). A diagnostic threshold criterion can include an assay achieving 100% specificity, such as achieving 100% specificity with reference to a control sample or set of samples. A diagnostic threshold criterion can include an assay achieving at least 99% specificity, such as achieving at least 99% specificity with reference to a control sample or set of samples. A diagnostic threshold criterion can include an assay achieving at least 98% specificity, such as achieving at least 98% specificity with reference to a control sample or set of samples. A diagnostic threshold criterion can include an assay achieving at least 90%, at least 91%, at least 92%, at least 93%, at least 94%, at least 95%, at least 96%, or at least 97% specificity, such as achieving at least 90%, at least 91%, at least 92%, at least 93%, at least 94%, at least 95%, at least 96%, or at least 97% specificity with reference to a control sample or set of samples. A diagnostic threshold criterion can include an assay achieving at least 99.0%, at least 99.1%, at least 99.2%, at least 99.3%, at least 99.4%, at least 99.5%, at least 99.6%, at least 99.7%, at least 99.8%, or at least 99.9% specificity, such as achieving at least 99.0%, at least 99.1%, at least 99.2%, at least 99.3%, at least 99.4%, at least 99.5%, at least 99.6%, at least 99.7%, at least 99.8%, or at least 99.9% specificity with reference to a control sample or set of samples.

A diagnostic threshold criterion can include an assay achieving a desired sensitivity. Sensitivity can be established with reference to a control sample or set of samples, such as correctly identifying as positive a set of control samples known to be positive for the disease (e.g., positive for SARS-CoV-2). A diagnostic threshold criterion can include an assay achieving 100% sensitivity, such as achieving 100% sensitivity with reference to a control sample or set of samples. A diagnostic threshold criterion can include an assay achieving at least 99% sensitivity, such as achieving at least 99% sensitivity with reference to a control sample or set of samples. A diagnostic threshold criterion can include an assay achieving at least 98% sensitivity, such as achieving at least 98% sensitivity with reference to a control sample or set of samples. A diagnostic threshold criterion can include an assay achieving at least 90%, at least 91%, at least 92%, at least 93%, at least 94%, at least 95%, at least 96%, or at least 97% sensitivity, such as achieving at least 90%, at least 91%, at least 92%, at least 93%, at least 94%, at least 95%, at least 96%, or at least 97% sensitivity with reference to a control sample or set of samples. A diagnostic threshold criterion can include an assay achieving at least 99.0%, at least 99.1%, at least 99.2%, at least 99.3%, at least 99.4%, at least 99.5%, at least 99.6%, at least 99.7%, at least 99.8%, or at least 99.9% sensitivity, such as achieving at least 99.0%, at least 99.1%, at least 99.2%, at least 99.3%, at least 99.4%, at least 99.5%, at least 99.6%, at least 99.7%, at least 99.8%, or at least 99.9% sensitivity with reference to a control sample or set of samples. A diagnostic threshold criterion can include an assay achieving at least 40%, at least 45%, at least 50%, at least 55%, at least 60%, at least 65%, at least 70%, at least 75%, at least 80%, at least 85%, at least 90%, or at least 95% sensitivity, such as achieving at least 40%, at least 45%, at least 50%, at least 55%, at least 60%, at least 65%, at least 70%, at least 75%, at least 80%, at least 85%, at least 90%, or at least 95% sensitivity with reference to a control sample or set of samples.

A diagnostic threshold criterion can include an assay achieving a desired specificity and a desired sensitivity. A diagnostic threshold criterion can include an assay achieving both 100% specificity and 100% sensitivity. A diagnostic threshold criterion can include an assay achieving 100% specificity and at least 99% sensitivity. A diagnostic threshold criterion can include an assay achieving 100% specificity and at least 98% sensitivity. A diagnostic threshold criterion can include an assay achieving 100% specificity and at least 90%, at least 91%, at least 92%, at least 93%, at least 94%, at least 95%, at least 96%, or at least 97% sensitivity. A diagnostic threshold criterion can include an assay achieving 100% specificity and at least 99.0%, at least 99.1%, at least 99.2%, at least 99.3%, at least 99.4%, at least 99.5%, at least 99.6%, at least 99.7%, at least 99.8%, or at least 99.9% sensitivity. A diagnostic threshold criterion can include an assay achieving 100% specificity and at least 40%, at least 45%, at least 50%, at least 55%, at least 60%, at least 65%, at least 70%, at least 75%, at least 80%, at least 85%, at least 90%, or at least 95% sensitivity.

A diagnostic threshold criterion can include an assay achieving both 99% specificity and 100% sensitivity. A diagnostic threshold criterion can include an assay achieving 99% specificity and at least 99% sensitivity. A diagnostic threshold criterion can include an assay achieving 99% specificity and at least 98% sensitivity. A diagnostic threshold criterion can include an assay achieving 99% specificity and at least 90%, at least 91%, at least 92%, at least 93%, at least 94%, at least 95%, at least 96%, or at least 97% sensitivity. A diagnostic threshold criterion can include an assay achieving 99% specificity and at least 99.0%, at least 99.1%, at least 99.2%, at least 99.3%, at least 99.4%, at least 99.5%, at least 99.6%, at least 99.7%, at least 99.8%, or at least 99.9% sensitivity. A diagnostic threshold criterion can include an assay achieving 99% specificity and at least 40%, at least 45%, at least 50%, at least 55%, at least 60%, at least 65%, at least 70%, at least 75%, at least 80%, at least 85%, at least 90%, or at least 95% sensitivity.

A diagnostic threshold criterion can include an assay achieving both 98% specificity and 100% sensitivity. A diagnostic threshold criterion can include an assay achieving 98% specificity and at least 99% sensitivity. A diagnostic threshold criterion can include an assay achieving 98% specificity and at least 98% sensitivity. A diagnostic threshold criterion can include an assay achieving 98% specificity and at least 90%, at least 91%, at least 92%, at least 93%, at least 94%, at least 95%, at least 96%, or at least 97% sensitivity. A diagnostic threshold criterion can include an assay achieving 98% specificity and at least 99.0%, at least 99.1%, at least 99.2%, at least 99.3%, at least 99.4%, at least 99.5%, at least 99.6%, at least 99.7%, at least 99.8%, or at least 99.9% sensitivity. A diagnostic threshold criterion can include an assay achieving 98% specificity and at least 40%, at least 45%, at least 50%, at least 55%, at least 60%, at least 65%, at least 70%, at least 75%, at least 80%, at least 85%, at least 90%, or at least 95% sensitivity.

A diagnostic threshold criterion can include an assay achieving both at least 90%, at least 91%, at least 92%, at least 93%, at least 94%, at least 95%, at least 96%, or at least 97% specificity and 100% sensitivity. A diagnostic threshold criterion can include an assay achieving at least 90%, at least 91%, at least 92%, at least 93%, at least 94%, at least 95%, at least 96%, or at least 97% specificity and at least 99% sensitivity. A diagnostic threshold criterion can include an assay achieving at least 90%, at least 91%, at least 92%, at least 93%, at least 94%, at least 95%, at least 96%, or at least 97% specificity and at least 98% sensitivity. A diagnostic threshold criterion can include an assay achieving at least 90%, at least 91%, at least 92%, at least 93%, at least 94%, at least 95%, at least 96%, or at least 97% specificity and at least 90%, at least 91%, at least 92%, at least 93%, at least 94%, at least 95%, at least 96%, or at least 97% sensitivity. A diagnostic threshold criterion can include an assay achieving at least 90%, at least 91%, at least 92%, at least 93%, at least 94%, at least 95%, at least 96%, or at least 97% specificity and at least 99.0%, at least 99.1%, at least 99.2%, at least 99.3%, at least 99.4%, at least 99.5%, at least 99.6%, at least 99.7%, at least 99.8%, or at least 99.9% sensitivity. A diagnostic threshold criterion can include an assay achieving at least 90%, at least 91%, at least 92%, at least 93%, at least 94%, at least 95%, at least 96%, or at least 97% specificity and at least 40%, at least 45%, at least 50%, at least 55%, at least 60%, at least 65%, at least 70%, at least 75%, at least 80%, at least 85%, at least 90%, or at least 95% sensitivity.

A diagnostic threshold criterion can include an assay achieving both at least 99.0%, at least 99.1%, at least 99.2%, at least 99.3%, at least 99.4%, at least 99.5%, at least 99.6%, at least 99.7%, at least 99.8%, or at least 99.9% specificity and 100% sensitivity. A diagnostic threshold criterion can include an assay achieving at least 99.0%, at least 99.1%, at least 99.2%, at least 99.3%, at least 99.4%, at least 99.5%, at least 99.6%, at least 99.7%, at least 99.8%, or at least 99.9% specificity and at least 99% sensitivity. A diagnostic threshold criterion can include an assay achieving at least 99.0%, at least 99.1%, at least 99.2%, at least 99.3%, at least 99.4%, at least 99.5%, at least 99.6%, at least 99.7%, at least 99.8%, or at least 99.9% specificity and at least 98% sensitivity. A diagnostic threshold criterion can include an assay achieving at least 99.0%, at least 99.1%, at least 99.2%, at least 99.3%, at least 99.4%, at least 99.5%, at least 99.6%, at least 99.7%, at least 99.8%, or at least 99.9% specificity and at least 90%, at least 91%, at least 92%, at least 93%, at least 94%, at least 95%, at least 96%, or at least 97% sensitivity. A diagnostic threshold criterion can include an assay achieving at least 99.0%, at least 99.1%, at least 99.2%, at least 99.3%, at least 99.4%, at least 99.5%, at least 99.6%, at least 99.7%, at least 99.8%, or at least 99.9% specificity and at least 99.0%, at least 99.1%, at least 99.2%, at least 99.3%, at least 99.4%, at least 99.5%, at least 99.6%, at least 99.7%, at least 99.8%, or at least 99.9% sensitivity. A diagnostic threshold criterion can include an assay achieving at least 99.0%, at least 99.1%, at least 99.2%, at least 99.3%, at least 99.4%, at least 99.5%, at least 99.6%, at least 99.7%, at least 99.8%, or at least 99.9% specificity and at least 40%, at least 45%, at least 50%, at least 55%, at least 60%, at least 65%, at least 70%, at least 75%, at least 80%, at least 85%, at least 90%, or at least 95% sensitivity.

Diagnostic threshold criterion used to evaluate a sample as considered positive for a disease can be the same or different than a diagnostic threshold criterion used to evaluate the severity of a disease. For example, a threshold criterion to diagnose a subject as positive and evaluate the severity of a disease can achieve a different specificity and/or sensitivity.

1.7.2. Computer

Many of the assays described herein (e.g., motif analysis, sequence alignment/clustering, NGS applications, etc.) typically require the use of a computer as they cannot be practically carried out by the human mind or by pen and paper alone. In general, a computer is adapted to execute a computer program for providing results, for example the results of determining nucleic acid sequences such as those sequences produced during a sequencing step or the results of an assessment step providing if the assay meets a quality control standard. Generally, the steps of determining the nucleic acid sequences and determining the results of the assessment step involve such a large number of computations, particularly given the number of sequences generally under consideration, that they are carried out by a computer system in order to be completed in a reasonable amount of time. They cannot be practically carried out by the human mind or by pen and paper alone. A computer can include at least one processor coupled to a chipset. Also coupled to the chipset can be a memory device, a memory controller hub, an input/output (I/O) controller hub, and/or a graphics adaptor. Various embodiments of the invention may be implemented as a computer program instructions stored in a non-transitory computer readable storage medium for execution by a processor of a computer system. The instructions define functions of the embodiments (including the methods described herein). Illustrative computer-readable storage media include, but are not limited to: (i) non-writable storage media (e.g., read-only memory devices within a computer such as CD-ROM disks readable by a CD-ROM drive, flash memory, ROM chips or any type of solid-state non-volatile semiconductor memory) on which information is permanently stored; and (ii) writable storage media (e.g., floppy disks within a diskette drive or hard-disk drive or any type of solid-state random-access semiconductor memory) on which alterable information is stored. A computer can include a means for programming the computer (i.e., providing computer program instructions), such as providing sequence alignment software or quality control assessment software. A computer can include a means for inputting information, such as sequences, including, but not limited to, a keyboard, a mouse, a touch-screen interface, or combinations thereof. A computer can include a means to display information and images, such as a graphics adaptor and display. A computer can include means to connect to other computers (e.g., computer networks), such as a network adaptor. Some portions of the description herein describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs, equivalent electrical circuits, or the like. The described operations and their associated modules may be embodied in software, firmware, hardware, or any combinations thereof. A quality control standard can be a ratio or percentage of unique peptide sequences specific present in a sample. The ratio or percentage can be relative to unique peptide sequences that are not specific to a sample (e.g., present as a result of sample contamination). The ratio or percentage can also be relative to the total number of any subset of nucleic acid sequences useful for establishing a quality control standard, such as sequences encoding a library of peptides, sequences encoding a control binding target, unique nucleic acid sequences, or combinations thereof.

A computer, as described herein, can be used to perform determination (e.g., sequencing) and assessment steps described herein.

The different methods described herein are not mutually exclusive.

### 1.8. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

1.8.1. Digital Serology Method

1.8.1.1. Overview

Digital Serology is a Next-generation Sequencing (NGS)-based assay similar to other biopanning assays in which peptide libraries are screened with human serum to map human antibody repertoires. The assay involves 4 main steps: 1) incubation of serum with the peptide library and affinity selection of library members expressing peptides that are specific to the antibody repertoire for each serum sample; 2) purification of plasmids that encode these peptides; 3) PCR amplification of the region of the plasmids encoding the peptides (amplicons) and barcoding of each sample with sample-specific primers (allowing samples to be pooled and sequenced together on a single NGS run); and 4) amplicon sequencing by NGS. Once the amplicons are sequenced, the data are demultiplexed and antibodies present in each serum sample are identified based on the peptide motifs to which they bind.

1.8.1.2. Methods

The methods described below are useful for the examples described herein but are not intended to be limiting. Methods useful for the present invention, e.g., digital serology including motif determination and motif analysis, are also described in more detail in Pantazes, et al. and international PCT application WO2017083874A1, each herein incorporated by reference in their entirety for all purposes.

### Serum Collection

The Yale discovery cohort had 157 serum samples, some representing serial draws from a total of 93 hospitalized patients who tested positive for COVID-19 by NAT testing were provided by Albert Ko, Professor of Epidemiology (Microbial Diseases) and of Medicine (Infectious Diseases), Yale University. Other serum samples used in motif identification and specificity testing (e.g., additional discovery or validation cohorts) were purchased from commercial sources. All Serimmune database samples used for specificity determination were pre-pandemic and presumed negative.

### Bacterial Surface Display Antibody Screen

A large, high-quality, bacterial-display, random, 12-mer peptide library composed of 8×10⁹ independent transformants, was constructed using trinucleotide oligos to eliminate stop codons and normalize amino acid usage frequencies. The 12-mer peptide library was displayed on E. coli via the N-terminus of a previously reported, engineered protein scaffold (eCPX), as described in more detail in Rice, et al., herein incorporated by reference for all it teaches. Vectors, methods, and other tools useful in the E. coli surface displayed peptide library are described in more detail in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference for all they teach.

The bacterial display peptide library was used to screen and isolate peptide binders to antibodies in individual serum samples through Magnetic Activated Cell Sorting (MACS). The MACS screen employed magnetic selection to enrich the library for antibody binding peptides as well as reduce the library size suitable for the subsequent screening steps. A frozen aliquot of the library containing 8×10¹⁰ cells (10-fold the diversity of the 12-mer X12 library) was thawed and inoculated into inoculated into multiple 2 L baffled flasks of 500 mL LB (10 g/L tryptone, 5 g/L yeast extract, and 10 g/L NaCl) supplemented with 34 μg/mL chloramphenicol. The flasks were incubated with vigorous shaking (300 rpm) at 37° C. until the cells grew to OD600=1.0. Peptide expression was induced with L-(+)-arabinose at a final concentration of 0.02% wt/vol for one hour. Cells were collected by centrifugation (3500 rcf for 30 minutes) and resuspended in cold phosphate buffered saline with 15% glycerol (PBSG). The cell suspension was divided into wells of a 96-deep-well plate to yield 10-fold the X12 diversity per well (8×10¹⁰ cells), frozen and stored at −80° C.

Prior to incubation with serum, cells were cleared of peptides that bind protein A/G by incubating cells with washed protein A/G magnetic beads (Pierce) at a ratio of one bead per 50 cells for 45 min. at 4° C. with gentle mixing. Magnetic separation for 5 min. (×2) was used to recover the unbound cells. Recovered cells from the supernatant are centrifuged, resuspended in diluted sera (1:25) and incubated for a one-hour primary incubation at 4° C. with orbital shaking (800 rpm). Following serum incubation, cells were then collected by centrifugation (3500 rcf for 7 minutes), the supernatant was removed, and the cell pellets were washed by resuspending fully in 750 uL PBS+0.05% Tween-20 (PBST). The cells were again collected by centrifugation (3500 rcf for 7 minutes) and the PBST supernatant was removed. Cell pellets were resuspended in 750 uL PBS and mixed thoroughly with 50 uL Protein A/G Sera-Mag SpeedBeads (GE Life Sciences, 17152104010350) (6.25% the beads' stock concentration) were used to identify IgG motifs. The plate was incubated for one hour at 4° C. with orbital shaking (800 rpm). Bead-bound cells were captured in the plate using a Magnum FLX 96-ring magnet (Alpaqua, A000400) until all beads were separated. Unbound cells in the supernatant were removed by gentle pipetting so as not to disturb the beads, leaving only those cells bound to A/G beads. To exclude residual unbound cells and wash non-antibody interactions of cells to beads, the beads were washed by removing from the magnet, resuspending in 750 uL PBST, and then returning to the magnet. The supernatant was removed by gentle pipetting after the beads were securely captured. Beads were washed 5×, and cells were resuspended in 750 uL LB with 34 μg/mL chloramphenicol and 0.2% wt/vol glucose directly in the 96-deep-well plate and grown overnight with shaking (300 rpm) at 37° C.

The following modifications were used to identify IgM motifs. In place of protein A/G beads, a biotinylated monoclonal antibody specific for human IgM [Jackson Immunoresearch, 709-066-073] was added to the mixture of library cells and serum, followed by cell capture on 100 ul Dynabeads MyOne streptavidin T1 coated magnetic beads (ThermoFisher Scientific, 65601).

### Next Generation Sequencing

After growth, cells were collected by centrifugation (3500 rcf for 10 minutes) and the supernatant was discarded. Plasmids were isolated in 96-well format using the Montage Plasmid MiniprepHTS Kit (MilliPore, LSKP09604) on a MultiscreenHTS Vacuum Manifold (MilliPore, MSVMHTS00) following the “Plasmid DNA-Full Lysate” protocol in the product literature. For amplicon preparation, two rounds of PCR were employed; the first round amplifies the variable “X12” peptide region of the plasmid DNA. The second round barcodes each patient amplicon library with sample-specific indexing primers for data demultiplexing after sequencing. KAPA HiFi HotStart ReadyMix (KAPA Biosystems, KK2612) was used as the polymerase master mix for all PCR steps. Plasmids (2.5 uL/well) were used as template for a first round PCR with 12.5 uL of KAPA ReadyMix and 5 uL each of 1 uM forward and reverse primers. The primers (Integrated DNA Technologies) contain annealing regions that flank the X12 sequence (indicated in bold) and adapter regions specific to the Illumina index primers used in the second round PCR.

Forward Primer (SEQ ID NO: 1):
TCGTCGGCAGCGTCAGATGTGTATAAGAGACAGVBHDVCCAGTCT
GGCCAGGG
Reverse Primer (SEQ ID NO: 2):
GTCTCGTGGGCTCGGAGATGTGTATAAGAGACAGGTGATGCCGTA
GTACTGG

A series of five degenerate bases in the forward primer, VBHDV (following IUPAC codes and italicized in the forward primer sequence above), provided base diversity for the first five reads of the sequencing on the NextSeq platform. The five base pairs were designed to be non-complementary to the template to avoid bias during primer annealing. To reduce non-specific products, a touchdown PCR protocol was used with an initial annealing temperature of 72° C. with a decrease of 0.5° C. per cycle for 14 cycles, followed by 10 cycles with annealing at 65° C. The 25 uL primary PCR product was purified using 30 uL Mag-Bind TotalPure NGS Beads (Omega Bio-Tek, M1378-02) according to the manufacturer's protocol. The second round PCR (8 cycles, 70° C. annealing temperature) was performed using Nextera XT index primers (Illumina, FC-131-2001) which introduce 8 base pair indices on the 5′ and 3′ termini of the amplicon for data demultiplexing of each sample screened. The PCR 1 product (5 uL) was used as a template for the second PCR with 5 uL each of forward and reverse indexing primers, 5 uL PCR grade water and 25 uL of KAPA ReadyMix. The PCR product (50 uL) was cleaned up with 56 uL Omega Mag-Bind TotalPure NGS Beads per reaction. A 96-well quantitation was performed using the Qubit dsDNA High Sensitivity assay (Invitrogen, Q32851) adapted for a microplate fluorimeter (Tecan SPECTRAFlour Plus) measuring fluorescence excitation at 485 nm and emission at 535 nm. Positive (100 ng) and negative (Ong) controls, included with the Qubit kit, were added to the plate as standards along with 2 uL of each PCR product diluted 1:100 for quantitation. The fluorescence data were used to calculate DNA concentration in each well based on the kit standards. To normalize the DNA and achieve equal loading of each patient sample on NGS, the DNA in each well was diluted with Tris HCl (pH 8.5, 10 mM) to 4 nM and an equal volume from each well was pooled in a Lo-Bind DNA tube for sequencing.

The sample pool was prepared for sequencing according to specifications of the Illumina NextSeq 500. Due to the low diversity in the adapter regions of the amplicon after the first five bases, PhiX Run Control (Illumina, FC-110-3001) was included at 40% of the final DNA pool. The pool was sequenced using a High Output v2, 75 cycle kit (Illumina, FC-404-2005).

### Disease-Specific Motif Discovery

Following NGS, the computational algorithm IMUNE was utilized to analyze the set of peptides (i.e., epitope repertoire) identified by sequencing and discover candidate SARS-CoV-2-specific peptide motifs. IMUNE compared 30 SARS-CoV-2 positive repertoires with 30 control repertoires. Multiple comparisons were run using different sets of unique subjects and different control groups. IMUNE identified patterns, defined as three to five amino acids interspersed with undefined residues (“X”) within a 10-amino acid window that were significantly enriched in disease samples but not enriched in controls. The enrichment of a pattern was quantified as the ratio of pattern observations in a sample to the expected observations taking into account the amino acid frequencies in the peptide library and total number of sequences for that sample. Patterns were selected if they were statistically significant (p<0.0001) in the specified percentage of disease samples and not statistically significant (p>0.5/# of controls) in the specified percentage of control samples.

To generate epitope motifs, significantly enriched patterns (˜10⁴-10⁵ patterns) were aligned and scored using a PAM30 substitution matrix as described previously. Unlike patterns, motifs may contain positions where multiple, often similar, amino acids are observed, indicated by brackets (e.g., [IVL] or [KRQ]. Pattern clustering generated ˜100s of motifs. Enrichments for motifs were calculated as the number of observations of a specific motif within a repertoire/the number expected by random chance.

### Phenotype Determination and Disease Diagnosis

Following NGS analysis, samples were analyzed for enrichment for each motif in a panel of peptide motifs that were previously identified as diagnostic motifs for specific diseases. The enrichment was calculated by dividing the number of observed instances for each motif by the number of expected instances. A z-score for each motif was calculated where each z-score indicates the enrichment value minus the mean enrichment for all samples divided by the standard deviation of all samples. To calculate a composite diagnostic score, the z-scores for each motif were summed for the specific disease. Diagnostic thresholds, e.g., whether a sample was considered to be positive for a disease, were established for each condition balancing sensitivity and specificity.

1.8.2. Determination of Motifs Associated with SARS-CoV-2

Antibody binding motifs for SARS-CoV-2 were determined using SERA digital serology, as described above. SARS-CoV-2 positive samples from Yale (n=160) (confirmed positive by NAT testing) were used to discover CoV-2 specific epitope motifs. IgG and IgM epitope motif panels are presented in Table 1A. IgG and IgM panel epitope motifs were selected that were enriched in ≥5% of disease and <2% of ˜1500 control repertoires, where a positive enrichment is defined as four standard deviations above the mean of the control group.

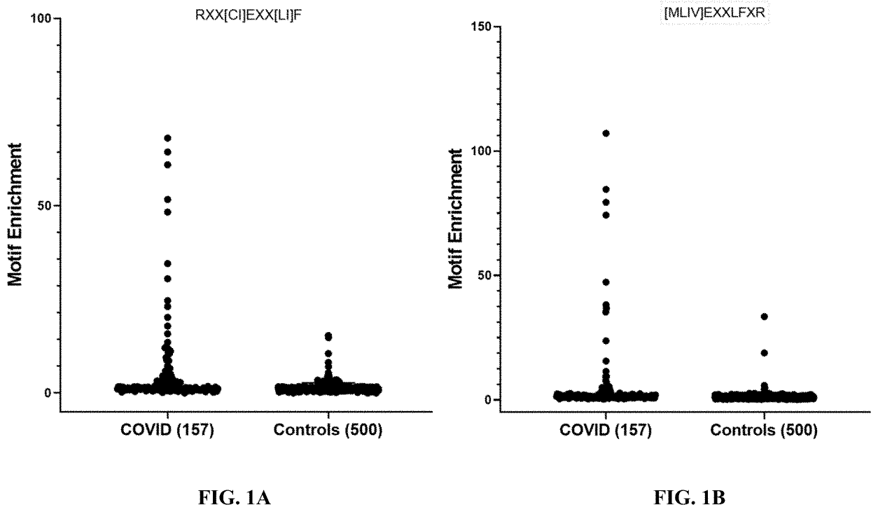

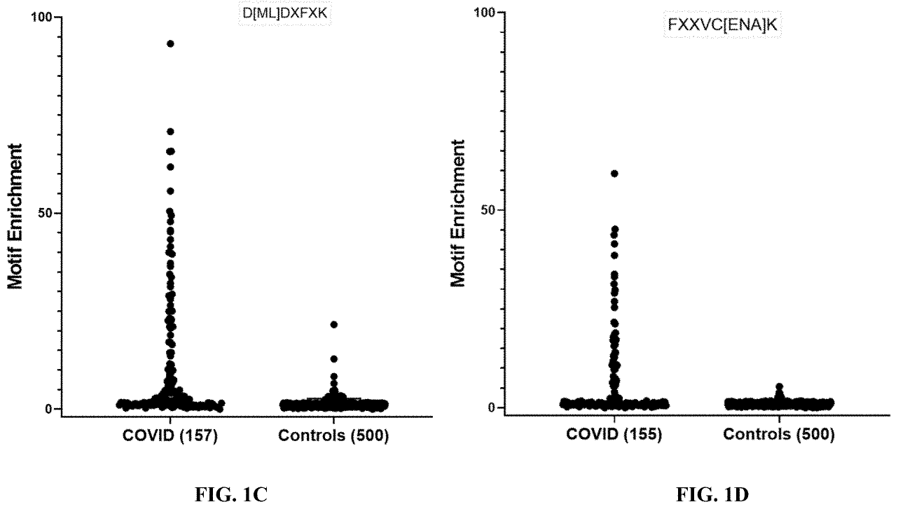

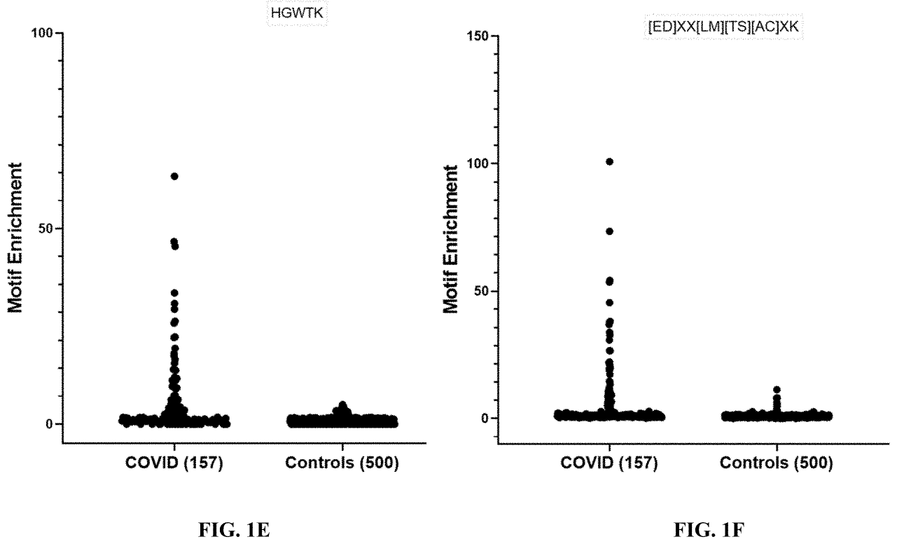

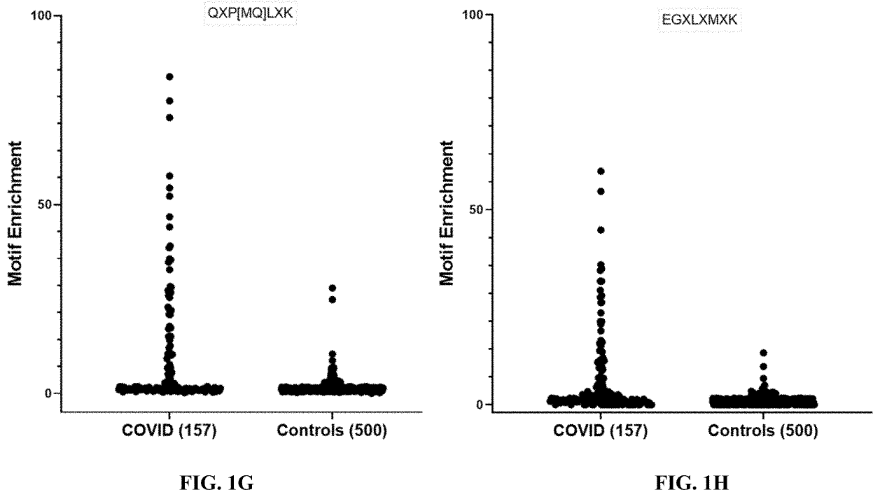

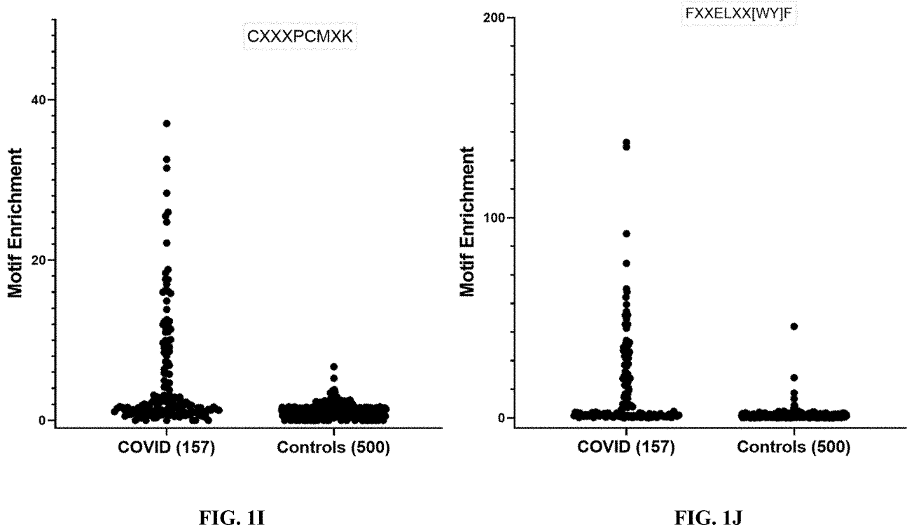

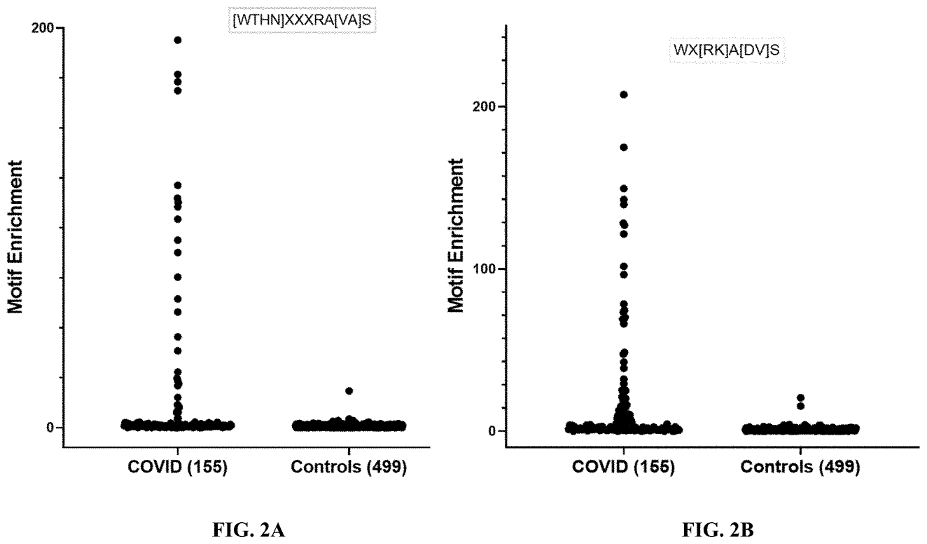

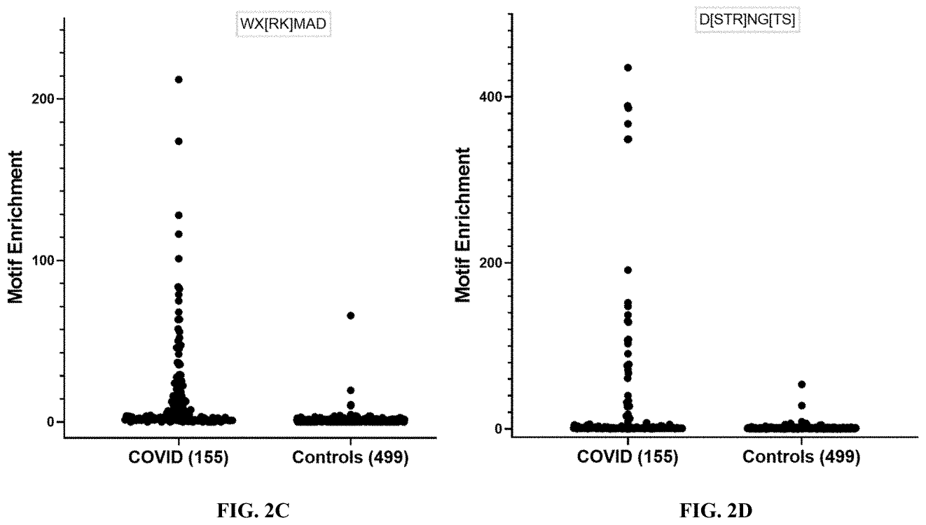

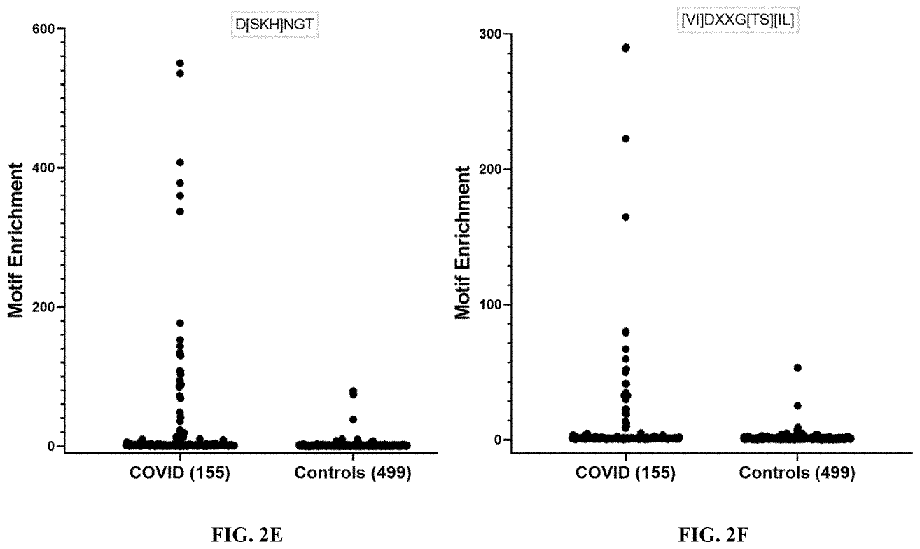

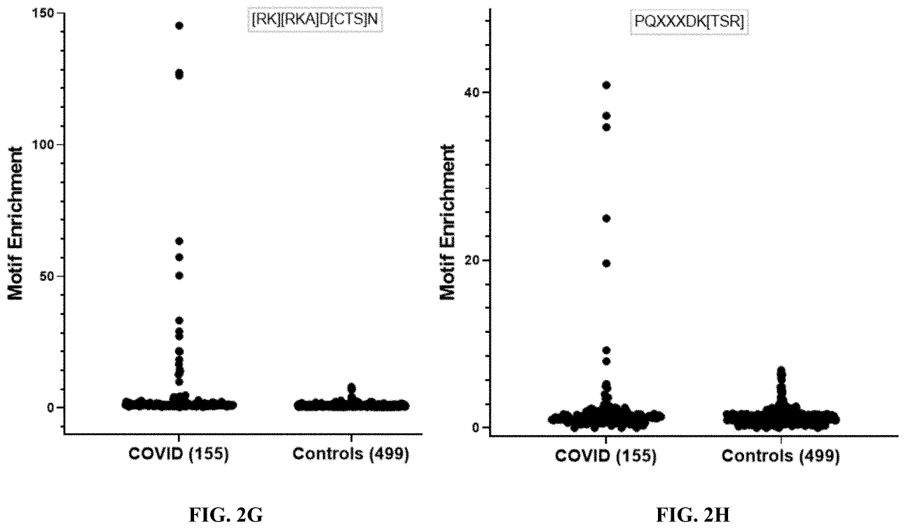

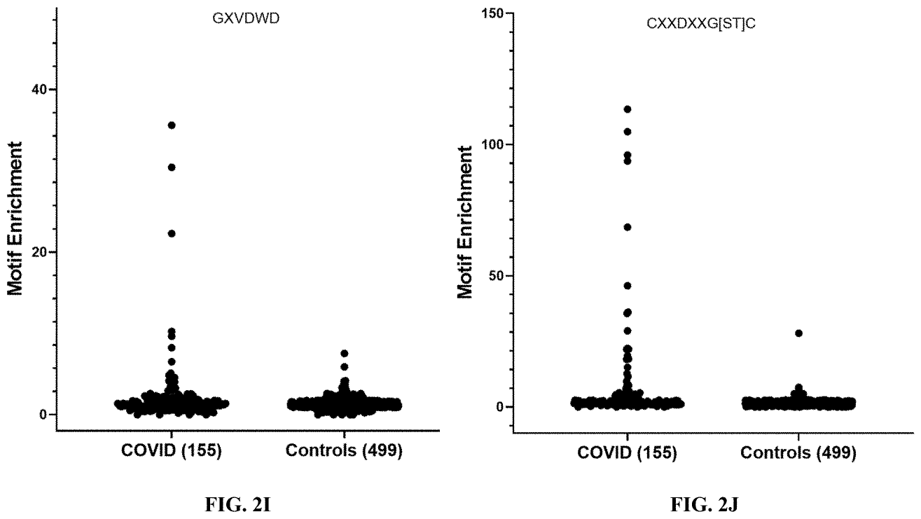

Diagnostic IgG and IgM epitope motif panels were also generated using ROC curve analysis to select diagnostic motifs that demonstrated the best performance for sensitivity and specificity, while also enriched in ≥5% of disease and <2% of ˜1500 control repertoires, and are shown in Table 2A and Table 2B. Enrichments for the diagnostic motifs in each panel are shown in FIG. 1 and FIG. 2. Individual motifs ranged from an average of 5-fold to 150-fold enrichment over the control group and each motif was present in a unique subset of patients. Motif were further categorized into specific motif classes if they shared a common specific amino acid or amino acid class in at least 3 specific positions and were known or likely to be derived from the same SARS-CoV-2 epitope. In summary, a panel of peptide motifs that serve as diagnostic biomarkers of SARS-CoV-2 infection was identified.

TABLE 1A
CoV-2 SERA Determined Epitope Motifs
IgG Motifs
LPFQQ (SEQ ID NO: 32)
KXX[AG]XQQ[FY] (SEQ ID NO: 71)
K[FY][LM]XFXQ (SEQ ID NO: 33)
KWXXFQQ (SEQ ID NO: 34)
K[WY]X[AP]FXQ (SEQ ID NO: 72)
[AC]K[FY]XXFXQ (SEQ ID NO: 73)
KWX[AP]FXQ (SEQ ID NO: 74)
[AE]KXXXF[ILV]Q (SEQ ID NO: 75)
WKXX[AP]FXQ (SEQ ID NO: 76)
RXPX[ST]L[DE] (SEQ ID NO: 77)
RXPX[ST]LE (SEQ ID NO: 78)
PXWR[IV]Y (SEQ ID NO: 79)
P[DE]WRXY (SEQ ID NO: 80)
[ITV]P[ST]WR (SEQ ID NO: 81)
RS[ILMV]AS (SEQ ID NO: 82)
RSVAS (SEQ ID NO: 83)
[EFY]TX[PS]LG (SEQ ID NO: 84)
ETXS[LM]G (SEQ ID NO: 85)
KTPP[ILM] (SEQ ID NO: 86)
R[EHS]XXEXXLF (SEQ ID NO: 87)
RXXQEXXLF (SEQ ID NO: 88)
RXX[CI]EXX[IL]F (SEQ ID NO: 89)
SXXEDXLF (SEQ ID NO: 90)
KXSXIEX[ILM] (SEQ ID NO: 91)
[ILMV]EXXLFXR (SEQ ID NO: 92)
FXXELXX[WY]F (SEQ ID NO: 15)
KEXLXXWF (SEQ ID NO: 93)
FEQXLXXXF (SEQ ID NO: 94)
D[LMY]SGI (SEQ ID NO: 95)
[ACP]XXXDXSGI (SEQ ID NO: 96)
[AP]Q[EG]XTXXK (SEQ ID NO: 97)
QGPTXP (SEQ ID NO: 98)
HXQCPXXXK (SEQ ID NO: 99)
[AET]QXPXXXK[HNR] (SEQ ID NO: 100)
LX[IL]LXXLN (SEQ ID NO: 101)
[FY]X[LV]LXXLN (SEQ ID NO: 102)
[LM]X[HR]LNX[ILM] (SEQ ID NO: 103)
FP[AP][MT]XXK (SEQ ID NO: 104)
[FY]PXTXPK (SEQ ID NO: 105)
KXQTX[ST]M (SEQ ID NO: 21)
LLPXXX[IL]D (SEQ ID NO: 106)
[ILM]LPXXXLD (SEQ ID NO: 107)
D[LM]DXFXK (SEQ ID NO: 108)
[DE]CX[AGM]XKXC (SEQ ID NO: 109)
DX[AC][GM][AFS]K (SEQ ID NO: 110)
DXAGXK (SEQ ID NO: 111)
DCX[AG]XKXC (SEQ ID NO: 112)
[FL][DN]XY[FY]XD (SEQ ID NO: 113)
KXX[IV]Q[FY]XD (SEQ ID NO: 114)
[GST]PXQYXD (SEQ ID NO: 115)
CXXXXCVMXK (SEQ ID NO: 116)
FXXV[MT]RK (SEQ ID NO: 117)
FXQVCXK (SEQ ID NO: 118)
EGXLXMXK (SEQ ID NO: 14)
[DE]GXLX[LM]XK (SEQ ID NO: 119)
WWXXL[LMT]XK (SEQ ID NO: 120)
WXX[IL]M[AL]K (SEQ ID NO: 121)
[DE]PX[LM]X[ACS]XK (SEQ ID NO: 122)
M[ST][ACTJAK (SEQ ID NO: 123)
MT[AT][AS][AK] (SEQ ID NO: 124)
[DNT]PXMX[AST]XK (SEQ ID NO: 125)
[FL]MX[AC]XKXXXI (SEQ ID NO: 126)
MW[AGJGK (SEQ ID NO: 127)
MTA[AT]K (SEQ ID NO: 128)
TPMXXXIST]XK (SEQ ID NO: 129)
STXXL[ST]XK (SEQ ID NO: 130)
[NST]P[FM]XXX[ST]XK (SEQ ID NO: 131)
[ST]PMXXAXXK (SEQ ID NO: 132)
[DNST]TXX[LM][ST]XK (SEQ ID NO: 133)
[AS]AX[FY]MXK (SEQ ID NO: 134)
SAXY[AMS]XK (SEQ ID NO: 135)
QXSFKXX[FMW] (SEQ ID NO: 136)
QX[NS]FKXX[FL] (SEQ ID NO: 137)
QXSFKXX[FW] (SEQ ID NO: 138)
KXXIPYXD (SEQ ID NO: 139)
K[DE]X[IV]PXXD (SEQ ID NO: 140)
KMXPWXQ (SEQ ID NO: 141)
CXLXWSXC (SEQ ID NO: 142)
YX[IR][FY]ML (SEQ ID NO: 143)
WF DE|XID (SEQ ID NO: 144)
R[FH]PEK (SEQ ID NO: 145)
FXE[HR]XK[PR] (SEQ ID NO: 146)
CXEX[ILM]LF (SEQ ID NO: 147)
PXWXXG[LM]D (SEQ ID NO: 148)
AXX[MS]RKP (SEQ ID NO: 149)
CXXFIAXC (SEQ ID NO: 36)
HGWTK (SEQ ID NO: 9)
KXG[ACT]PXXXXP (SEQ ID NO: 150)
YWXYFXK (SEQ ID NO: 151)
CXXYWX[AC]XXK (SEQ ID NO: 152)
CXVXPXMXK (SEQ ID NO: 153)
YCXAXKXC (SEQ ID NO: 154)
KXXGPXXLY (SEQ ID NO: 39)
KXP[FW]CP (SEQ ID NO: 155)
[AV]XRW[FRY]F (SEQ ID NO: 156)
IgM Motifs
WX[RK]MAD (SEQ ID NO: 50)
RMADXXR (SEQ ID NO: 51)
RMADX[AHR] (SEQ ID NO: 157)
WAXMAD (SEQ ID NO: 158)
WX[RK]A[DV]S (SEQ ID NO: 42)
[WTHN]XXXRA[VA]S (SEQ ID NO: 43)
[FY][RK]A[DAV][TS] (SEQ ID NO: 44)
CXXC[KRJAXS (SEQ ID NO: 45)
[AS][KR]MAXS (SEQ ID NO: 159)
[FW][FY]XAXS[FY] (SEQ ID NO: 160)
[KR]AVS[DEGNQ] (SEQ ID NO: 161)
PXXRAXSXX[HPW] (SEQ ID NO: 162)
AVSX[MNQ][HL] (SEQ ID NO: 163)
PXXXRAXSXG (SEQ ID NO: 164)
[KR]AVXX[GR]X[FY] (SEQ ID NO: 165)
[KRJAXSXX[ST]X[HRS] (SEQ ID NO: 166)
[KR]AXSXX[RW]M (SEQ ID NO: 167)
[IR][FNW][KRJAXS (SEQ ID NO: 168)
KAXSXXXXXQ (SEQ ID NO: 169)
[AT]XAVSXXX[HNY] (SEQ ID NO: 170)
[HQ]X[AST][KRJAXS (SEQ ID NO: 171)
IXXAVSX[AS] (SEQ ID NO: 172)
KAXSXXXY (SEQ ID NO: 173)
[AP][KR]AXSX[DENS] (SEQ ID NO: 174)
[KR]AXSMXXR (SEQ ID NO: 175)
[KR]AXSXXX[HP][HY] (SEQ ID NO: 176)
[IMN][LM]X[KR]AXS (SEQ ID NO: 177)
[FY]XKAXSXXY (SEQ ID NO: 178)
[KR]AXSX[LM][DE] (SEQ ID NO: 179)
[FY]XRAMS (SEQ ID NO: 180)
Y[KR]XD[ST][NY] (SEQ ID NO: 181)
[FW]XRRDXG (SEQ ID NO: 182)
WX[KR][AKR]D[ST] (SEQ ID NO: 183)
FXR[AK]DXXXP (SEQ ID NO: 184)
[FY][KR][KR]DXXXP (SEQ ID NO: 185)
[KR][KR]DCXXXC (SEQ ID NO: 186)
[KR][AKR]D[CST]N (SEQ ID NO: 187)
LX[AP]R[RKA]D (SEQ ID NO: 46)
FX[RK][RK]DT (SEQ ID NO: 47)
[RK][RKA]D[CTS]N (SEQ ID NO: 48)
[YF][RK]R[DA]XXX[PVR] (SEQ ID NO: 49)
PNG[TS]XXP (SEQ ID NO: 61)
DSNG[ST] (SEQ ID NO: 188)
[RY]ADXNG (SEQ ID NO: 189)
[AIV]XXNG[ST]X[FLW] (SEQ ID NO: 190)
[ITV]XXNG[ST][ILV] (SEQ ID NO: 191)
D[STRING[TS] (SEQ ID NO: 54)
D[SKH]NGT (SEQ ID NO: 55)
[VI]DXXG[TS][IL] (SEQ ID NO: 56)
DXXG[TS][FWY][MV] (SEQ ID NO: 57)
[AI]DXXGTX[CHT] (SEQ ID NO: 58)
CXXDXXG[ST]C (SEQ ID NO: 59)
DXNX[TS]XK[YF] (SEQ ID NO: 60)
D[MR]NX[ST][LW] (SEQ ID NO: 192)
DXNX[ST][CY][TW] (SEQ ID NO: 193)
PX[ANP][QR]XDK (SEQ ID NO: 194)
PQXXXDK[TSR] (SEQ ID NO: 52)
[IV]XXX[QR][DN]KXH (SEQ ID NO: 53)
[AT]RA[VL]X[NTE] (SEQ ID NO: 64)
IPGS[FY] (SEQ ID NO: 195)
CSTIXC (SEQ ID NO: 196)
A[FIY]SG[GHS] (SEQ ID NO: 197)
RD[ST]XXPXR (SEQ ID NO: 198)

TABLE 2A
Diagnostic CoV-2 IgG Epitope Motifs

| | Motif | Sensitivity All | Validation | SEQ ID |
|---|---|---|---|---|
| IgG Motif | Class | Cohorts (344) | Specificity (1500) | NO. |
| RXX[CI]EXX\|LI]F | 1 | 18.6% | 99.1% | 4 |
| RXXQE[DNJXXF | 1 | 11.6% | 99.3% | 5 |
| [MLIV]EXXLFXR | | 10.5% | 99.3% | 6 |
| D[ML]DXFXK | | 45.3% | 98.8% | 7 |
| FXXVC[ENA]K | | 33.7% | 98.9% | 8 |
| HGWTK | | 25.6% | 99.5% | 9 |
| [ED]XX[LM][TS][AC]XK | 2 | 27.6% | 99.2% | 10 |
| D[PA]X[ML]X[STA]XK | 2 | 26.7% | 99.1% | 11 |
| MT[TA][AS][KA] | 2 | 18.6% | 99.5% | 12 |
| M[TS][ACTJAK | 2 | 21.5% | 99.7% | 13 |
| EGXLXMXK | | 34.6% | 99.3% | 14 |
| FXXELXX[WY]F | 3 | 44.5% | 99.1% | 15 |
| [FY]XX[QE]LXXWF | 3 | 38.4% | 98.9% | 16 |
| QX[PT]C[LM]XK | 4 | 32.6% | 97.9% | 17 |
| QXP[MQ]LXK | 4 | 18.3% | 99.6% | 18 |
| QXXC[LM]XKXXF | 4 | 25.6% | 98.4% | 19 |
| CXXXPCMXK | | 34.3% | 99.3% | 20 |
| KXQTX[ST]M | | 16.9% | 98.6% | 21 |
| QX[AS]MXPK | | 23.5% | 99.2% | 22 |
| [TS]PMXXAXXK | | 31.4% | 99.7% | 23 |
| STXXL[TS]XK | | 27.0% | 99.2% | 24 |
| QX[SN]FKXX[FL] | | 19.5% | 98.1% | 25 |
| RXPX[TS]L[ED] | | 20.6% | 99.3% | 26 |
| P[EDT]WRXY | 5 | 16.6% | 98.7% | 27 |
| PXWR[VI]Y | 5 | 18.0% | 98.9% | 28 |
| DXSGIXXX[DGESN] | 6 | 14.0% | 99.1% | 29 |
| [APC]XXXDXSGI | 6 | 12.8% | 99.5% | 30 |
| KTPP[LMI] | | 10.8% | 99.3% | 31 |
| LPFQQ | | 10.2% | 99.5% | 32 |
| K[FY][LM]XFXQ | 7 | 16.9% | 99.9% | 33 |
| KWXXFQQ | 7 | 19.2% | 99.1% | 34 |
| [CAJK[FY]XXFXQ | 7 | 18.0% | 99.6% | 35 |
| CXXFIAXC | | 13.7% | 100.0% | 36 |
| CXXYWX[CA]XXK | | 24.7% | 98.7% | 37 |
| [LMI]LPXXXLD | | 17.2% | 99.3% | 38 |
| KXXGPXXLY | | 27.3% | 99.1% | 39 |
| QGPT[LI] | | 23.0% | 99.3% | 40 |
| KXG[TCA]PXXXXP | | 20.6% | 99.7% | 41 |

TABLE 2B
Diagnostic CoV-2 IgM Epitope Motifs

| | | Discovery | Validation | SEQ |
|---|---|---|---|---|
| | Motif | Sensitivity | Specificity | ID |
| IgM Motif | class | (155) | (1497) | NO. |
| WX[RK]A[DV]S | 1 | 40.6% | 99.0% | 42 |
| [WTHN]XXXRA[VA]S | 1 | 19.4% | 99.7% | 43 |
| [FY][RK]A[DAV][TS] | 1 | 28.4% | 98.7% | 44 |
| CXXC[KRJAXS | 1 | 22.6% | 99.3% | 45 |
| LX[AP]R[RKA]D | 2 | 16.8% | 99.0% | 46 |
| FX[RK][RK]DT | 2 | 17.4% | 99.5% | 47 |
| [RK][RKA]D[CTS]N | 2 | 18.7% | 98.8% | 48 |
| [YF][RK]R[DA]XXX | 2 | 18.1% | 98.7% | 49 |
| [PVR] | | | | |
| WX[RK]MAD | 3 | 31.6% | 99.4% | 50 |
| RMADXXR | 3 | 32.3% | 99.2% | 51 |
| PQXXXDK[TSR] | | 6.5% | 98.5% | 52 |
| [IV]XXX[QR][DNJKXH | | 5.8% | 98.7% | 53 |
| D[STRING[TS] | 4 | 16.8% | 99.8% | 54 |
| D[SKH]NGT | 4 | 23.2% | 99.3% | 55 |
| [VI]DXXG[TS][IL] | 4 | 14.8% | 99.7% | 56 |
| DXXG[TS][FWY][MV] | 4 | 13.5% | 99.6% | 57 |
| [AI]DXXGTX[CHT] | 4 | 14.2% | 99.0% | 58 |
| CXXDXXG[STIC | 4 | 12.3% | 99.6% | 59 |
| DXNX[TS]XK[YF] | 4 | 7.7% | 99.5% | 60 |
| PNG[TS]XXP | 4 | 15.5% | 99.0% | 61 |
| GXVDWD | | 4.5% | 99.6% | 62 |
| CXPXCXEXXW | | 4.5% | 99.9% | 63 |
| [AT]RA[VL]X[NTE] | | 7.1% | 99.8% | 64 |

1.8.3. Validation of Diagnostic Motifs Associated with SARS-CoV-2

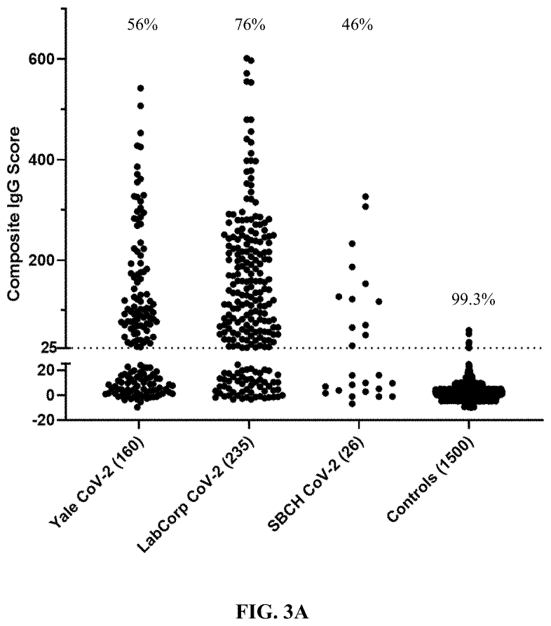

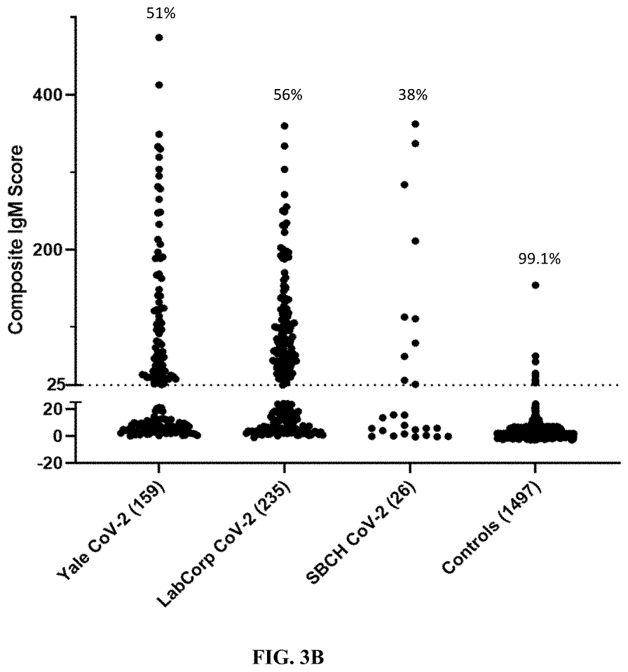

A “Validation” cohort of confirmed SARS CoV-2 samples (positive by NAT testing) from Santa Barbara Collage Hospital (SBCH) (26) and LabCorp (235) were used to validate that the identified diagnostic motifs (Tables 2A and Table 2B3) accurately reflected SARS CoV-2 specific antibodies rather than antibodies to another disease to which a subset of the discovery cohort had been exposed. A cohort of 1500 pre-pandemic control repertoires from the Serimmune database not used in discovery or downselection of motifs was also used to assess panel specificity, here referring to the ability to correctly identify as negative samples considered to be negative for SARS-CoV-2. Motif enrichments for each sample were converted to z-scores and summed to generate a composite diagnostic score. A cut off of 25 was chosen that yielded 100% specificity on the discovery control set (not shown) and took into consideration the natural separation within the COVID positive repertoires. Dot plots showing diagnostic scores for the IgG and IgM panels on all cohorts are shown in FIG. 3A (IgG) and FIG. 3B (IgM). The sensitivities of the SERA IgG and IgM panels are shown in Table 3. The SERA IgG and IgM panels together were 73% and 82% sensitive on the Yale discovery cohort and LabCorp validation cohort, respectively. SERA panel sensitivities for the SBCH cohort were lower than other cohorts tested. SBCH cohort samples were generally collected at an earlier disease stage (majority collected ≤8 days after symptom onset), potentially before a subject mounted an immune response, and may explain the lower sensitivities for the cohort. The IgG and IgM panels demonstrated a combined specificity of 98.1% on a validation cohort of 1500 samples from the Serimmune database pre COVID outbreak.

TABLE 3
Panel sensitivity by cohort

| Cohort | SERA IgG | SERA IgM | Overall IgG + M |
|---|---|---|---|
| Yale | 56 | 52 | 73 |
| Labcorp | 76 | 56 | 82 |
| SBCH | 46 | 38 | 58 |
| Overall | 67 | 53 | 77 |

The percent positivity on samples relative to days of symptom onset was determined (Table 4). The IgM immune response preceded the IgG response, with 50% positivity at 1-5 days post symptom onset. By 16-20 days post symptom onset, 91% of samples were positive by IgG and/or IgM.

TABLE 4
Panel sensitivity by days since symptom onset

| Days since | | | |
|---|---|---|---|
| symptom onset | SERA IgG | SERA IgM | Overall IgG + M |
| 1-5 | 19% | 50% | 50% |
| 6-10 | 53% | 45% | 73% |
| 11-15 | 79% | 64% | 88% |
| 16-20 | 82% | 68% | 91% |
| >20 | 50% | 63% | 75% |

Diagnostic capabilities of the SERA assay were further compared against an ELISA. Of the subset of Yale cohort sera for which serological test results were available (n=126), 67% were serologically positive by Spike S1 glycoprotein (ACROBiosystems #S1N-C52H3) IgG and/or IgM ELISA. The LabCorp cohort was 77% positive for CoV-2 antibodies using a Spike glycoprotein S1 IgG ELISA (IgM not performed). The SBCH cohort was not tested by ELISA. SERA results indicate the assays demonstrated improved or comparable sensitivities to ELISA performance. The Yale cohort was 72% positive by SERA IgG/M compared with 67% positive by a combined IgG and IgM S1 ELISA. Both SERA IgG and IgG S1 ELISA were 77% positive on the Labcorp cohort. On the Yale cohort, SERA identified 13 samples as positive that were ELISA S1 negative, and ELISA identified 7 samples as positive that were SERA negative. For the LabCorp cohort, SERA and ELISA each identified 14 samples that were negative by the other test.

In summary, the identified panels of peptide motifs (Tables 2A and Table 2B) were validated to serve as diagnostic biomarkers of SARS-CoV-2 infection, and the results indicated SERA performance was as good or better than ELISA.

1.8.4. Selection of Motifs for Optimal Sensitivity

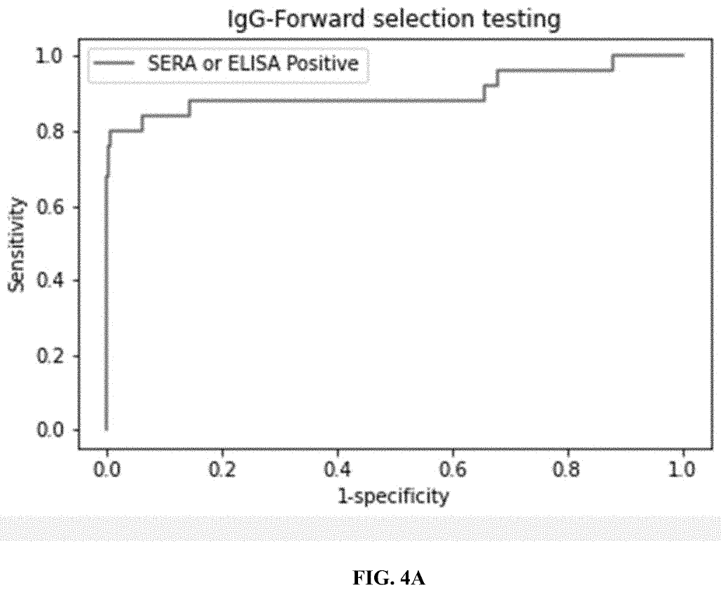

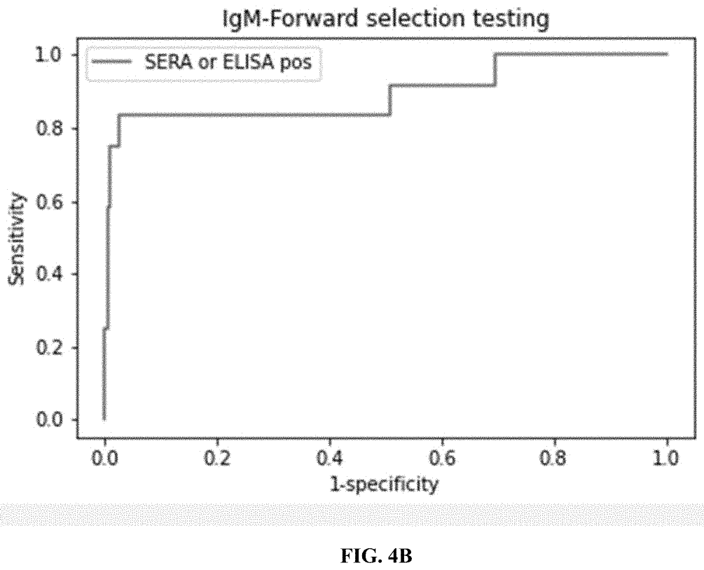

To identify the set of IgG and IgM SARS CoV-2 specific epitope motifs that achieve a balance between maintaining specificity while optimizing sensitivity, an algorithm was used to combine the determined diagnostic motifs (Tables 1B and 2B) sequentially, summing the z-scores of each motif for each subject at each iteration to maximize the sensitivity in the SARS CoV-2 cohort while maintaining the specificity at or above 99%. The motif panel performance was trained on a set of 59 disease and 1046 pre-pandemic controls and tested on a set of 25 disease and 449 pre-pandemic controls. All SARS CoV-2 samples were confirmed positive by nucleic acid and serological testing. ROC curves illustrating the iterative performance analysis are presented in FIG. 4A for IgG and FIG. 4B for IgM motifs and demonstrate analysis of combined motifs improved sensitivity performance while maintaining specificity.

1.8.5. Additional Diagnostic Panel Generation

IgG motifs in Table 1 and Tables 2A/2B were generated using different strategies. Accordingly, the two were compared and additional IgG motifs from Table 1 demonstrating the highest enrichments and sensitivities were added, or motif residue optionality expanded, to generate expanded diagnostic marker panels, as shown in Table 5.

TABLE 5
Expanded Diagnostic Motif Panel

| | Motif |
|---|---|
| IgG Motifs | Groups |
| KXX[AG]XQQ[FY] (SEQ ID NO: 71) | 1 |
| K[FY][LM]XFXQ (SEQ ID NO: 33) | 1 |
| KWXXFQQ (SEQ ID NO: 34) | 1 |
| K[WY]X[AP]FXQ (SEQ ID NO: 72) | 1 |
| [AC]K[FY]XXFXQ (SEQ ID NO: 73) | 1 |
| RXPX[ST]L[DE] (SEQ ID NO: 77) | 2 |
| RXPX[ST]LE (SEQ ID NO: 78) | 2 |
| PXWR[IV]Y (SEQ ID NO: 79) | 3 |
| P[EDT]WRXY (SEQ ID NO: 27) | 3 |
| [ITV]P[ST]WR (SEQ ID NO: 81) | 3 |
| RS[ILMV]AS (SEQ ID NO: 82) | 4 |
| [RK]S[ML]AS (SEQ ID NO: 199) | 4 |
| [EFY]TX[PS]LG (SEQ ID NO: 84) | 5 |
| ETXS[LM]G (SEQ ID NO: 85) | 5 |
| [FI]AXEXDK (SEQ ID NO: 200) | |
| KTPP[ILM] (SEQ ID NO: 86) | |
| R[EHS]XXEXXLF (SEQ ID NO: 87) | 6 |
| RXXQEXXLF (SEQ ID NO: 88) | 6 |
| RXX[CI]EXX[IL]F (SEQ ID NO: 89) | 6 |
| SXXEDXLF (SEQ ID NO: 90) | 6 |
| RXXQE[DN]XXF (SEQ ID NO: 5) | 6 |
| KXSXIEX[ILM] (SEQ ID NO: 91) | |
| [ILMV]EXXLFXR (SEQ ID NO: 92) | |
| FXXELXX[WY]F (SEQ ID NO: 15) | 7 |
| KEXLXXWF (SEQ ID NO: 93) | 7 |
| [FY]XX[QE]LXXWF (SEQ ID NO: 16) | 7 |
| D[LMY]SGI (SEQ ID NO: 95) | 8 |
| [ACP]XXXDXSGI (SEQ ID NO: 96) | 8 |
| DXSGIXXX[DGESN] (SEQ ID NO: 29) | 8 |
| [AP]Q[EG]XTXXK (SEQ ID NO: 97) | 9 |
| QGPTXP (SEQ ID NO: 98) | 9 |
| QGPT[LI] (SEQ ID NO: 40) | 9 |
| HXQCPXXXK (SEQ ID NO: 99) | 9 |
| QX[PT]C[LM]XK (SEQ ID NO: 17) | 9 |
| QXP[MQ]LXK (SEQ ID NO: 18) | 9 |
| QXXC[LM]XKXXF (SEQ ID NO: 19) | 9 |
| [DNJAAXAL (SEQ ID NO: 201) | |
| LX[IL]LXXLN (SEQ ID NO: 101) | 10 |
| [FY]X[LV]LXXLN (SEQ ID NO: 102) | 10 |
| [LM]X[HR]LNX[ILM] (SEQ ID NO: 103) | 10 |
| [GS][AS]AXXX[KR]K (SEQ ID NO: 202) | |
| FP[AP][MT]XXK (SEQ ID NO: 104) | 11 |
| [FY]PXTXPK (SEQ ID NO: 105) | 11 |
| KXQTX[ST]M (SEQ ID NO: 21) | |
| LLPXXX[IL]D (SEQ ID NO: 106) | 12 |
| [ILM]LPXXXLD (SEQ ID NO: 107) | 12 |
| D[LM]DXFXK (SEQ ID NO: 108) | |
| [DE]CX[AGM]XKXC (SEQ ID NO: 109) | 13 |
| DX[AC][GM][AFS]K (SEQ ID NO: 110) | 13 |
| DXAGXK (SEQ ID NO: 111) | 13 |
| [FL][DN]XY[FY]XD (SEQ ID NO: 113) | 14 |
| KXX[IV]Q[FY]XD (SEQ ID NO: 114) | 14 |
| [GST]PXQYXD (SEQ ID NO: 115) | 14 |
| CXXXXCVMXK (SEQ ID NO: 116) | 15 |
| FXXV[MT]RK (SEQ ID NO: 117) | 15 |
| FXQVCXK (SEQ ID NO: 118) | 15 |
| FXXVC[ENA]K (SEQ ID NO: 8) | 15 |
| EGXLXMXK (SEQ ID NO: 14) | 16 |
| [DE]GXLX[LM]XK (SEQ ID NO: 119) | 16 |
| WWXXL[LMT]XK (SEQ ID NO: 120) | 17 |
| WXX[IL]M[AL]K (SEQ ID NO: 121) | 17 |
| [DE]PX[LM]X[ACS]XK (SEQ ID NO: 122) | 18 |
| M[ST][ACTJAK (SEQ ID NO: 123) | 18 |
| MT[AT][AS][AK] (SEQ ID NO: 124) | 18 |
| [ED]XX[LM][TS][AC]XK (SEQ ID NO: 10) | 18 |
| D[PA]X[ML]X[STA]XK (SEQ ID NO: 11) | 18 |
| TPMXXX[ST]XK (SEQ ID NO: 129) | 19 |
| STXXL[ST]XK (SEQ ID NO: 130) | 19 |
| [NST]P[FM]XXX[ST]XK (SEQ ID NO: 131) | 19 |
| [ST]PMXXAXXK (SEQ ID NO: 132) | 19 |
| [AS]AX[FY]MXK (SEQ ID NO: 134) | 20 |
| SAXY[AMS]XK (SEQ ID NO: 135) | 20 |
| QXSFKXX[FMW] (SEQ ID NO: 136) | 21 |
| QX[NS]FKXX[FL] (SEQ ID NO: 137) | 21 |
| KXXIPYXD (SEQ ID NO: 139) | 22 |
| K[DE]X[IV]PXXD (SEQ ID NO: 140) | 22 |
| KMXPWXQ (SEQ ID NO: 141) | |
| CXLXWSXC (SEQ ID NO: 142) | |
| YX[IR][FY]ML (SEQ ID NO: 143) | |
| WF[DE]XID (SEQ ID NO: 144) | |
| R[FH]PEK (SEQ ID NO: 145) | |
| FXE[HR]XK[PR] (SEQ ID NO: 146) | |
| CXEX[ILM]LF (SEQ ID NO: 147) | |
| PXWXXG[LM]D (SEQ ID NO: 148) | |
| AXX[MS]RKP (SEQ ID NO: 149) | |
| CXXFIAXC (SEQ ID NO: 36) | |
| HGWTK (SEQ ID NO: 9) | |
| KXG[ACT]PXXXXP (SEQ ID NO: 150) | |
| YWXYFXK (SEQ ID NO: 151) | |
| CXXYWX[AC]XXK (SEQ ID NO: 152) | |
| CXVXPXMXK (SEQ ID NO: 153) | 23 |
| CXXXPCMXK (SEQ ID NO: 20) | 23 |
| YCXAXKXC (SEQ ID NO: 154) | |
| KXXGPXXLY (SEQ ID NO: 39) | |
| KXP[FW]CP (SEQ ID NO: 155) | |
| QX[AS]MXPK (SEQ ID NO: 22) | |

1.8.6. SARS-CoV-2 Disease Severity Assessment Panels

Curated cohorts of COVID cases for which disease severity was known were analyzed to determine motifs associated with disease severity. Cases were considered mild if they exhibited few or no symptoms and were not hospitalized, moderate if they were hospitalized but not intubated or in ICU and severe if they were intubated, in ICU or expired from symptoms of COVID.

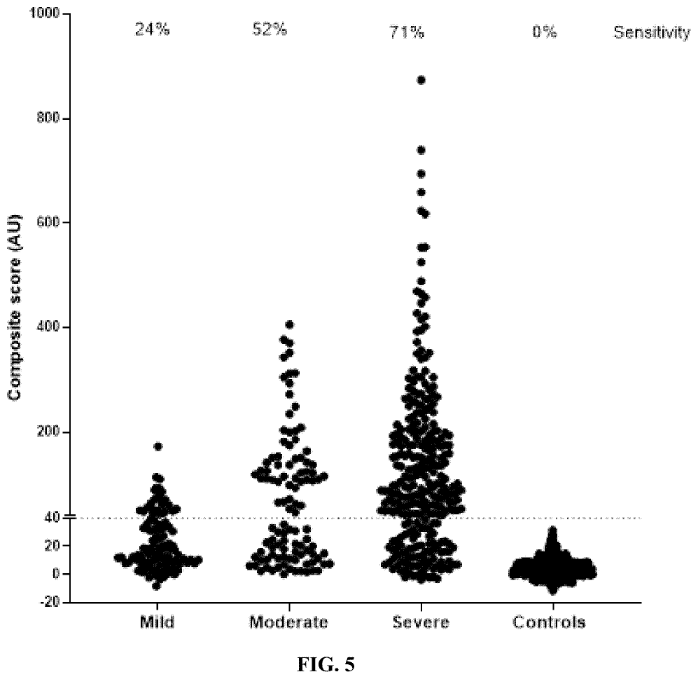

In a first approach, the expanded diagnostic IgG panel (Table 5) was used and those generally demonstrating a 2-fold or greater change in enrichment between mild and moderate/severe samples as well as low sensitivity in mild samples. Table 6 presents the selected motifs and corresponding sensitivities across samples. As shown in FIG. 5, summing the normalized motif enrichments (z-scores) for motifs that were present in <10% of mild or asymptomatic disease (bolded sequences) and using a severity z-score cut-off of 40 yielded a severity classifier score capable of distinguishing mild cases (24% above cutoff) from moderate and severe disease (52% and 71% above cutoff, respectively).

TABLE 6
SARS-COV-2 Disease Severity Panel 1

| | Sensitivity | | |
|---|---|---|---|
| | Mild | Moderate | Severe |
| SARS-COV-2 IgG Motif | n = 116 | n = 105 | n = 289 |
| PXWR[IV]Y (SEQ ID NO: 79) | 8.62% | 20.00% | 77.16% |
| P[EDT]WRXY (SEQ ID NO: 27) | 6.03% | 16.19% | 78.89% |
| [ITV]P[ST]WR (SEQ ID NO: 81) | 0.86% | 7.62% | 91.00% |
| RS[ILMV]AS (SEQ ID NO: 82) | 18.97% | 30.48% | 54.67% |
| [EFY]TX[PS]LG (SEQ ID NO: 84) | 1.72% | 5.71% | 91.70% |
| ETXS[LM]G (SEQ ID NO: 85) | 0.86% | 3.81% | 92.39% |
| [FI]AXEXDK (SEQ ID NO: 200) | 11.21% | 25.71% | 74.74% |
| KTPP[ILM] (SEQ ID NO: 86) | 1.72% | 9.52% | 83.74% |
| DILMY]SGI (SEQ ID NO: 95) | 6.03% | 12.38% | 76.47% |
| [ACP]XXXDXSGI (SEQ ID NO: 96) | 4.31% | 11.43% | 79.58% |
| K[DS]G[ILVIXW (SEQ ID NO: 203) | 0.86% | 3.81% | 87.20% |
| [AP]Q[EG]XTXXK (SEQ ID NO: 97) | 6.03% | 22.86% | 74.74% |
| QGPTXP (SEQ ID NO: 98) | 7.76% | 20.00% | 77.16% |
| QGPT[LI] (SEQ ID NO: 40) | 11.21% | 21.90% | 72.32% |
| HXQCPXXXK (SEQ ID NO: 99) | 6.90% | 21.90% | 74.74% |
| QX[PT]C[LM]XK (SEQ ID NO: 17) | 18.10% | 34.29% | 61.94% |
| QXP[MQ]LXK (SEQ ID NO: 18) | 7.76% | 19.05% | 77.16% |
| QXXC[LM]XKXXF (SEQ ID NO: 19) | 10.34% | 29.52% | 71.97% |
| LX[IL]LXXLN (SEQ ID NO: 101) | 3.45% | 16.19% | 69.90% |
| [FY]X[LV]LXXLN (SEQ ID NO: 102) | 3.45% | 10.48% | 81.66% |
| [GS][AS]AXXX[KR]K (SEQ ID NO: 202) | 2.59% | 16.19% | 76.82% |
| FP[AP][MT]XXK (SEQ ID NO: 104) | 1.72% | 10.48% | 79.58% |
| [FY]PXTXPK (SEQ ID NO: 105) | 5.17% | 9.52% | 84.43% |
| KXQTX[ST]M (SEQ ID NO: 21) | 8.62% | 15.24% | 79.24% |
| LLPXXX[IL]D (SEQ ID NO: 106) | 12.07% | 18.10% | 75.43% |
| [ILM]LPXXXLD (SEQ ID NO: 107) | 9.48% | 16.19% | 78.20% |
| [DE]CX[AGM]XKXC (SEQ ID NO: 109) | 10.34% | 16.19% | 79.24% |
| [FL][DN]XY[FY]XD (SEQ ID NO: 113) | 2.59% | 7.62% | 79.24% |
| KXX[IV]Q[FY]XD (SEQ ID NO: 114) | 4.31% | 12.38% | 83.74% |
| CXXXXCVMXK (SEQ ID NO: 116) | 12.93% | 37.14% | 60.55% |
| FXXV[MT]RK (SEQ ID NO: 117) | 9.48% | 32.38% | 62.98% |
| FXQVCXK (SEQ ID NO: 118) | 8.62% | 34.29% | 62.28% |
| FXXVC[ENA]K (SEQ ID NO: 8) | 7.76% | 33.33% | 60.21% |
| EGXLXMXK (SEQ ID NO: 14) | 8.62% | 37.14% | 57.44% |
| [DE]GXLX[LM]XK (SEQ ID NO: 119) | 5.17% | 32.38% | 62.63% |
| WWXXL[LMT]XK (SEQ ID NO: 120) | 9.48% | 35.24% | 63.32% |
| WXX[IL]M[AL]K (SEQ ID NO: 121) | 7.76% | 28.57% | 69.20% |
| [DE]PX[LM]X[ACS]XK (SEQ ID NO: 122) | 6.03% | 32.38% | 62.63% |
| MIST][ACT]AK (SEQ ID NO: 123) | 2.59% | 22.86% | 75.43% |
| MT[AT][AS][AK] (SEQ ID NO: 124) | 3.45% | 18.10% | 76.82% |
| [ED]XX[LM][TS][AC]XK (SEQ ID NO: 10) | 4.31% | 28.57% | 66.09% |
| D[PAJX[ML]X[STAJXK (SEQ ID NO: 11) | 2.59% | 27.62% | 67.47% |
| TPMXXX[ST]XK (SEQ ID NO: 129) | 3.45% | 24.76% | 71.28% |
| STXXL[ST]XK (SEQ ID NO: 130) | 6.03% | 29.52% | 67.13% |
| [NST]P[FM]XXX[ST]XK (SEQ ID NO: 131) | 4.31% | 39.05% | 66.78% |
| [ST]PMXXAXXK (SEQ ID NO: 132) | 2.59% | 37.14% | 65.05% |
| [AS]AX[FY]MXK (SEQ ID NO: 134) | 3.45% | 20.00% | 75.09% |
| SAXY[AMS]XK (SEQ ID NO: 135) | 3.45% | 19.05% | 71.97% |
| QXSFKXX[FMW] (SEQ ID NO: 136) | 3.45% | 24.76% | 80.97% |
| QX[NS]FKXX[FL] (SEQ ID NO: 137) | 3.45% | 24.76% | 80.97% |
| K[DE]X[IV]PXXD (SEQ ID NO: 140) | 5.17% | 8.57% | 82.01% |
| KMXPWXQ (SEQ ID NO: 141) | 4.31% | 6.67% | 81.31% |
| CXLXWSXC (SEQ ID NO: 142) | 1.72% | 5.71% | 81.66% |
| YX[IR][FY]ML (SEQ ID NO: 143) | 6.90% | 7.62% | 82.35% |
| WF[DE]XID (SEQ ID NO: 144) | 3.45% | 12.38% | 87.20% |
| R[FH]PEK (SEQ ID NO: 145) | 0.00% | 19.05% | 88.24% |
| FXE[HR]XK[PR] (SEQ ID NO: 146) | 3.45% | 8.57% | 87.20% |
| PXWXXG[LM]D (SEQ ID NO: 148) | 8.62% | 17.14% | 82.01% |
| AXX[MS]RKP (SEQ ID NO: 149) | 2.59% | 15.24% | 79.58% |
| CXXFIAXC (SEQ ID NO: 36) | 4.31% | 13.33% | 82.70% |
| HGWTK (SEQ ID NO: 9) | 1.72% | 26.67% | 69.90% |
| KXG[ACT]PXXXXP (SEQ ID NO: 150) | 4.31% | 14.29% | 69.90% |
| YWXYFXK (SEQ ID NO: 151) | 5.17% | 20.95% | 75.78% |
| CXXYWX[AC]XXK (SEQ ID NO: 152) | 5.17% | 28.57% | 71.97% |
| CXVXPXMXK (SEQ ID NO: 153) | 2.59% | 16.19% | 76.82% |
| CXXXPCMXK (SEQ ID NO: 20) | 8.62% | 40.00% | 58.13% |
| YCXAXKXC (SEQ ID NO: 154) | 4.31% | 23.81% | 69.20% |
| KXXGPXXLY (SEQ ID NO: 39) | 6.90% | 20.95% | 62.63% |
| KXP[FW]CP (SEQ ID NO: 155) | 12.07% | 29.52% | 61.25% |
| QX[AS]MXPK (SEQ ID NO: 22) | 11.21% | 21.90% | 70.59% |

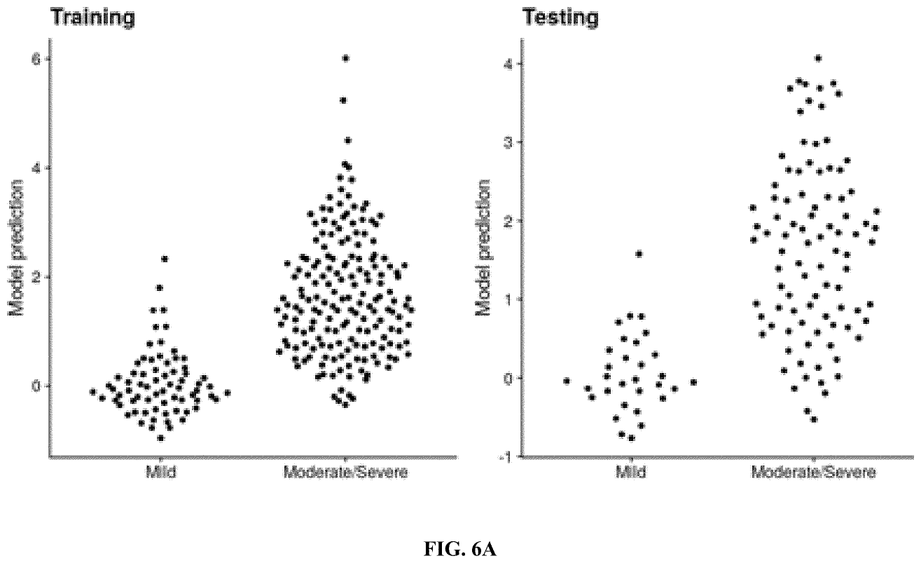

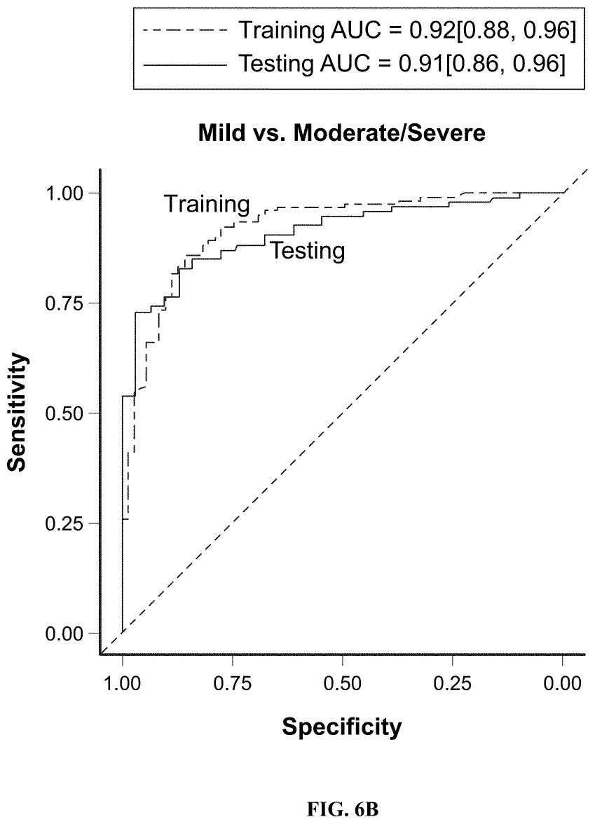

In a second approach for generating a disease severity assessment panel, a LASSO algorithm examined the IgG and IgM motifs presented in Table 1 to determine motifs with predictive power to assess severity. The curated samples cohorts of COVID cases were split into training (⅔) and testing (⅓) groups without regard for the severity of disease with the expectation that ⅔ of all severity classes ended up in the training group. Table 7 presents the motifs with non-zero coefficients representing those motifs that, collectively, yield the optimal disease severity assessment predictive power. Performance of the severity motif panel is shown in FIG. 6A (groupings of mild vs. moderate/severe) and FIG. 6B (ROC curve).

TABLE 7
SARS-COV-2 Disease Severity Panel 2

[AS]AX[FY]MXK (SEQ ID NO: 134)
[DE]PX[LM]X[ACS]XK (SEQ ID NO: 122)
HGWTK (SEQ ID NO: 9)
R[FH]PEK (SEQ ID NO: 145)
[AP]Q[EG]XTXXK (SEQ ID NO: 97)
[FL][DN]XY[FY]XD (SEQ ID NO: 113)
AXX[MS]RKP (SEQ ID NO: 149)
D[LM]DXFXK (SEQ ID NO: 108)
EGXLXMXK (SEQ ID NO: 14)
KXQTX[ST]M (SEQ ID NO: 21)
KXXGPXXLY (SEQ ID NO: 39)
LX[IL]LXXLN (SEQ ID NO: 101)
IPGS[FY] (SEQ ID NO: 195)
A[FIY]SG[GHS] (SEQ ID NO: 197)

Accordingly, the data demonstrate panels of highly disease specific SARS-CoV-2 epitope motifs that were capable of classifying Covid disease as mild, moderate, and/or severe.

1.8.7. PIWAS Analysis

A PIWAS analysis was performed where each 12mer identified by SERA, as described above, was deconstructed into constituent kmers (where k=5 and k=6 consecutive amino acids). PIWAS analysis is described in Haynes et al. (Protein-based Immunome Wide Association Studies (PIWAS) for the discovery of significant disease-associated antigens. bioRxiv 2020.03.18.997759 (2020)), herein incorporated by reference for all purposes. Shown in Table A is: the respective SARS-CoV-2 Protein (column 1 “Prot”; refer to Table B [FASTA sequences] for sequences and Uniprot identifiers); the position index within the respective protein (column 2 “Ind”) and refers to an effective 10-mer window for each position (e.g., Index 1 refers to the window of amino acids 1-10 of a respective protein, Index 2 to amino acids 2-11, etc.); the average PIWAS value across 579 COVID patients (column 3 “AvgVal”); and the number of COVID patients with a PIWAS value >4 at this position (column 4 “Out”). The average PIWAS value was calculated by averaging PIWAS values at the amino acid index across the COVID patients. As an illustrative example, reference in Table A to SPIKE protein with an Index Value of 1 refers to peptide sequence(s) MFVFLVLLPL (SEQ ID NO:204), which is calculated from the kmers MFVFL (SEQ ID NO:205), FVFLV (SEQ ID NO:206), VFLVL (SEQ ID NO:207), FLVLL (SEQ ID NO:208), LVLLP (SEQ ID NO:209), MFVFLV (SEQ ID NO:210), FVFLVL (SEQ ID NO:211), VFLVLL (SEQ ID NO:212), FLVLLP (SEQ ID NO:213), LVLLPL (SEQ ID NO:214) having an average PIWAS value of −0.0877 with 0 outliers

TABLE B
SARS-COV-2 Proteins Analyzed by PIWAS
“AP3A” SARS2 Protein 3a (SEQ ID NO: 65)
MDLFMRIFTIGTVTLKQGEIKDATPSDFVRATATIPIQASLPFGWLIVGVALLAVFQSASKIITLKKRWQ
LALSKGVHFVCNLLLLFVTVYSHLLLVAAGLEAPFLYLYALVYFLQSINFVRIIMRLWLCWKCRSKNP
LLYDANYFLCWHTNCYDYCIPYNSVTSSIVITSGDGTTSPISEHDYQIGGYTEKWESGVKDCVVLHSY
FTSDYYQLYSTQLSTDTGVEHVTFFIYNKIVDEPEEHVQIHTIDGSSGVVNPVMEPIYDEPTTTTSVPL
“NCAP″ SARS2 Nucleoprotein (SEQ ID NO: 66)
MSDNGPQNQRNAPRITFGGPSDSTGSNQNGERSGARSKQRRPQGLPNNTASWFTALTQHGKEDLKFP
RGQGVPINTNSSPDDQIGYYRRATRRIRGGDGKMKDLSPRWYFYYLGTGPEAGLPYGANKDGIIWVA
TEGALNTPKDHIGTRNPANNAAIVLQLPQGTTLPKGFYAEGSRGGSQASSRSSSRSRNSSRNSTPGSSR
GTSPARMAGNGGDAALALLLLDRLNQLESKMSGKGQQQQGQTVTKKSAAEASKKPRQKRTATKAY
NVTQAFGRRGPEQTQGNFGDQELIRQGTDYKHWPQIAQFAPSASAFFGMSRIGMEVTPSGTWLTYTG
AIKLDDKDPNFKDQVILLNKHIDAYKTFPPTEPKKDKKKKADETQALPQRQKKQQTVTLLPAADLDD
FSKQLQQSMSSADSTQA
“NS7A″ SARS2 Protein 7a (SEQ ID NO: 67)
MKIILFLALITLATCELYHYQECVRGTTVLLKEPCSSGTYEGNSPFHPLADNKFALTCFSTQFAFACPD
GVKHVYQLRARSVSPKLFIRQEEVQELYSPIFLIVAAIVFITLCFTLKRKTE
“ORF9B″ SARS2 Protein 9b (SEQ ID NO: 68)
MDPKISEMHPALRLVDPQIQLAVTRMENAVGRDQNNVGPKVYPIILRLGSPLSLNMARKTLNSLEDK
AFQLTPIAVQMTKLATTEELPDEFVVVTVK
“R1AB″ SARS2 Replicase polyprotein 1ab (SEQ ID NO: 69)
MESLVPGFNEKTHVQLSLPVLQVRDVLVRGFGDSVEEVLSEARQHLKDGTCGLVEVEKGVLPQLEQP
YVFIKRSDARTAPHGHVMVELVAELEGIQYGRSGETLGVLVPHVGEIPVAYRKVLLRKNGNKGAGG
HSYGADLKSFDLGDELGTDPYEDFQENWNTKHSSGVTRELMRELNGGAYTRYVDNNFCGPDGYPLE
CIKDLLARAGKASCTLSEQLDFIDTKRGVYCCREHEHEIAWYTERSEKSYELQTPFEIKLAKKFDTFNG
ECPNFVFPLNSIIKTIQPRVEKKKLDGFMGRIRSVYPVASPNECNQMCLSTLMKCDHCGETSWQTGDF
VKATCEFCGTENLTKEGATTCGYLPQNAVVKIYCPACHNSEVGPEHSLAEYHNESGLKTILRKGGRTI
AFGGCVFSYVGCHNKCAYWVPRASANIGCNHTGVVGEGSEGLNDNLLEILQKEKVNINIVGDFKLNE
EIAIILASFSASTSAFVETVKGLDYKAFKQIVESCGNFKVTKGKAKKGAWNIGEQKSILSPLYAFASEA
ARVVRSIFSRTLETAQNSVRVLQKAAITILDGISQYSLRLIDAMMFTSDLATNNLVVMAYITGGVVQL
TSQWLTNIFGTVYEKLKPVLDWLEEKFKEGVEFLRDGWEIVKFISTCACEIVGGQIVTCAKEIKESVQT
FFKLVNKFLALCADSIIIGGAKLKALNLGETFVTHSKGLYRKCVKSREETGLLMPLKAPKEIIFLEGETL
PTEVLTEEVVLKTGDLQPLEQPTSEAVEAPLVGTPVCINGLMLLEIKDTEKYCALAPNMMVTNNTFTL
KGGAPTKVTFGDDTVIEVQGYKSVNITFELDERIDKVLNEKCSAYTVELGTEVNEFACVVADAVIKTL
QPVSELLTPLGIDLDEWSMATYYLFDESGEFKLASHMYCSFYPPDEDEEEGDCEEEEFEPSTQYEYGT
EDDYQGKPLEFGATSAALQPEEEQEEDWLDDDSQQTVGQQDGSEDNQTTTIQTIVEVQPQLEMELTP
VVQTIEVNSFSGYLKLTDNVYIKNADIVEEAKKVKPTVVVNAANVYLKHGGGVAGALNKATNNAM
QVESDDYIATNGPLKVGGSCVLSGHNLAKHCLHVVGPNVNKGEDIQLLKSAYENFNQHEVLLAPLLS
AGIFGADPIHSLRVCVDTVRTNVYLAVFDKNLYDKLVSSFLEMKSEKQVEQKIAEIPKEEVKPFITESK
PSVEQRKQDDKKIKACVEEVTTTLEETKFLTENLLLYIDINGNLHPDSATLVSDIDITFLKKDAPYIVGD
VVQEGVLTAVVIPTKKAGGTTEMLAKALRKVPTDNYITTYPGQGLNGYTVEEAKTVLKKCKSAFYIL
PSIISNEKQEILGTVSWNLREMLAHAEETRKLMPVCVETKAIVSTIQRKYKGIKIQEGVVDYGARFYFY
TSKTTVASLINTLNDLNETLVTMPLGYVTHGLNLEEAARYMRSLKVPATVSVSSPDAVTAYNGYLTS
SSKTPEEHFIETISLAGSYKDWSYSGQSTQLGIEFLKRGDKSVYYTSNPTTFHLDGEVITFDNLKTLLSL
REVRTIKVFTTVDNINLHTQVVDMSMTYGQQFGPTYLDGADVTKIKPHNSHEGKTFYVLPNDDTLRV
EAFEYYHTTDPSFLGRYMSALNHTKKWKYPQVNGLTSIKWADNNCYLATALLTLQQIELKFNPPALQ
DAYYRARAGEAANFCALILAYCNKTVGELGDVRETMSYLFQHANLDSCKRVLNVVCKTCGQQQTT
LKGVEAVMYMGTLSYEQFKKGVQIPCTCGKQATKYLVQQESPFVMMSAPPAQYELKHGTFTCASEY
TGNYQCGHYKHITSKETLYCIDGALLTKSSEYKGPITDVFYKENSYTTTIKPVTYKLDGVVCTEIDPKL
DNYYKKDNSYFTEQPIDLVPNQPYPNASFDNFKFVCDNIKFADDLNQLTGYKKPASRELKVTFFPDLN
GDVVAIDYKHYTPSFKKGAKLLHKPIVWHVNNATNKATYKPNTWCIRCLWSTKPVETSNSFDVLKS
EDAQGMDNLACEDLKPVSEEVVENPTIQKDVLECNVKTTEVVGDIILKPANNSLKITEEVGHTDLMA
AYVDNSSLTIKKPNELSRVLGLKTLATHGLAAVNSVPWDTIANYAKPFLNKVVSTTTNIVTRCLNRVC
TNYMPYFFTLLLQLCTFTRSTNSRIKASMPTTIAKNTVKSVGKFCLEASFNYLKSPNFSKLINIIIWFLLL
SVCLGSLIYSTAALGVLMSNLGMPSYCTGYREGYLNSTNVTIATYCTGSIPCSVCLSGLDSLDTYPSLE
TIQITISSFKWDLTAFGLVAEWFLAYILFTRFFYVLGLAAIMQLFFSYFAVHFISNSWLMWLIINLVQM
APISAMVRMYIFFASFYYVWKSYVHVVDGCNSSTCMMCYKRNRATRVECTTIVNGVRRSFYVYANG
GKGFCKLHNWNCVNCDTFCAGSTFISDEVARDLSLQFKRPINPTDQSSYIVDSVTVKNGSIHLYFDKA
GQKTYERHSLSHFVNLDNLRANNTKGSLPINVIVFDGKSKCEESSAKSASVYYSQLMCQPILLLDQAL
VSDVGDSAEVAVKMFDAYVNTFSSTFNVPMEKLKTLVATAEAELAKNVSLDNVLSTFISAARQGFV
DSDVETKDVVECLKLSHQSDIEVTGDSCNNYMLTYNKVENMTPRDLGACIDCSARHINAQVAKSHNI
ALIWNVKDFMSLSEQLRKQIRSAAKKNNLPFKLTCATTRQVVNVVTTKIALKGGKIVNNWLKQLIKV
TLVFLFVAAIFYLITPVHVMSKHTDFSSEIIGYKAIDGGVTRDIASTDTCFANKHADFDTWFSQRGGSY
TNDKACPLIAAVITREVGFVVPGLPGTILRTTNGDFLHFLPRVFSAVGNICYTPSKLIEYTDFATSACVL
AAECTIFKDASGKPVPYCYDTNVLEGSVAYESLRPDTRYVLMDGSIIQFPNTYLEGSVRVVTTFDSEY
CRHGTCERSEAGVCVSTSGRWVLNNDYYRSLPGVFCGVDAVNLLTNMFTPLIQPIGALDISASIVAGG
IVAIVVTCLAYYFMRFRRAFGEYSHVVAFNTLLFLMSFTVLCLTPVYSFLPGVYSVIYLYLTFYLTNDV
SFLAHIQWMVMFTPLVPFWITIAYIICISTKHFYWFFSNYLKRRVVFNGVSFSTFEEAALCTFLLNKEM
YLKLRSDVLLPLTQYNRYLALYNKYKYFSGAMDTTSYREAACCHLAKALNDFSNSGSDVLYQPPQT
SITSAVLQSGFRKMAFPSGKVEGCMVQVTCGTTTLNGLWLDDVVYCPRHVICTSEDMLNPNYEDLLI
RKSNHNFLVQAGNVQLRVIGHSMQNCVLKLKVDTANPKTPKYKFVRIQPGQTFSVLACYNGSPSGV
YQCAMRPNFTIKGSFLNGSCGSVGFNIDYDCVSFCYMHHMELPTGVHAGTDLEGNFYGPFVDRQTA
QAAGTDTTITVNVLAWLYAAVINGDRWFLNRFTTTLNDFNLVAMKYNYEPLTQDHVDILGPLSAQT
GIAVLDMCASLKELLQNGMNGRTILGSALLEDEFTPFDVVRQCSGVTFQSAVKRTIKGTHHWLLLTIL
TSLLVLVQSTQWSLFFFLYENAFLPFAMGIIAMSAFAMMFVKHKHAFLCLFLLPSLATVAYFNMVYM
PASWVMRIMTWLDMVDTSLSGFKLKDCVMYASAVVLLILMTARTVYDDGARRVWTLMNVLTLVY
KVYYGNALDQAISMWALIISVTSNYSGVVTTVMFLARGIVFMCVEYCPIFFITGNTLQCIMLVYCFLG
YFCTCYFGLFCLLNRYFRLTLGVYDYLVSTQEFRYMNSQGLLPPKNSIDAFKLNIKLLGVGGKPCIKV
ATVQSKMSDVKCTSVVLLSVLQQLRVESSSKLWAQCVQLHNDILLAKDTTEAFEKMVSLLSVLLSM
QGAVDINKLCEEMLDNRATLQAIASEFSSLPSYAAFATAQEAYEQAVANGDSEVVLKKLKKSLNVAK
SEFDRDAAMQRKLEKMADQAMTQMYKQARSEDKRAKVTSAMQTMLFTMLRKLDNDALNNIINNA
RDGCVPLNIIPLTTAAKLMVVIPDYNTYKNTCDGTTFTYASALWEIQQVVDADSKIVQLSEISMDNSP
NLAWPLIVTALRANSAVKLQNNELSPVALRQMSCAAGTTQTACTDDNALAYYNTTKGGRFVLALLS
DLQDLKWARFPKSDGTGTIYTELEPPCRFVTDTPKGPKVKYLYFIKGLNNLNRGMVLGSLAATVRLQ
AGNATEVPANSTVLSFCAFAVDAAKAYKDYLASGGQPITNCVKMLCTHTGTGQAITVTPEANMDQE
SFGGASCCLYCRCHIDHPNPKGFCDLKGKYVQIPTTCANDPVGFTLKNTVCTVCGMWKGYGCSCDQ
LREPMLQSADAQSFLNRVCGVSAARLTPCGTGTSTDVVYRAFDIYNDKVAGFAKFLKTNCCRFQEKD
EDDNLIDSYFVVKRHTFSNYQHEETIYNLLKDCPAVAKHDFFKFRIDGDMVPHISRQRLTKYTMADL
VYALRHFDEGNCDTLKEILVTYNCCDDDYFNKKDWYDFVENPDILRVYANLGERVRQALLKTVQFC
DAMRNAGIVGVLTLDNQDLNGNWYDFGDFIQTTPGSGVPVVDSYYSLLMPILTLTRALTAESHVDTD
LTKPYIKWDLLKYDFTEERLKLFDRYFKYWDQTYHPNCVNCLDDRCILHCANFNVLFSTVFPPTSFGP
LVRKIFVDGVPFVVSTGYHFRELGVVHNQDVNLHSSRLSFKELLVYAADPAMHAASGNLLLDKRTTC
FSVAALTNNVAFQTVKPGNFNKDFYDFAVSKGFFKEGSSVELKHFFFAQDGNAAISDYDYYRYNLPT
MCDIRQLLFVVEVVDKYFDCYDGGCINANQVIVNNLDKSAGFPFNKWGKARLYYDSMSYEDQDALF
AYTKRNVIPTITQMNLKYAISAKNRARTVAGVSICSTMTNRQFHQKLLKSIAATRGATVVIGTSKFYG
GWHNMLKTVYSDVENPHLMGWDYPKCDRAMPNMLRIMASLVLARKHTTCCSLSHRFYRLANECA
QVLSEMVMCGGSLYVKPGGTSSGDATTAYANSVFNICQAVTANVNALLSTDGNKIADKYVRNLQHR
LYECLYRNRDVDTDFVNEFYAYLRKHFSMMILSDDAVVCFNSTYASQGLVASIKNFKSVLYYQNNV
FMSEAKCWTETDLTKGPHEFCSQHTMLVKQGDDYVYLPYPDPSRILGAGCFVDDIVKTDGTLMIERF
VSLAIDAYPLTKHPNQEYADVFHLYLQYIRKLHDELTGHMLDMYSVMLTNDNTSRYWEPEFYEAMY
TPHTVLQAVGACVLCNSQTSLRCGACIRRPFLCCKCCYDHVISTSHKLVLSVNPYVCNAPGCDVTDV
ERLKLFAAETLKATEETFKLSYGIATVREVLSDRELHLSWEVGKPRPPLNRNYVFTGYRVTKNSKVQI
GEYTFEKGDYGDAVVYRGTTTYKLNVGDYFVLTSHTVMPLSAPTLVPQEHYVRITGLYPTLNISDEFS
SNVANYQKVGMQKYSTLQGPPGTGKSHFAIGLALYYPSARIVYTACSHAAVDALCEKALKYLPIDKC
SRIIPARARVECFDKFKVNSTLEQYVFCTVNALPETTADIVVFDEISMATNYDLSVVNARLRAKHYVY
IGDPAQLPAPRTLLTKGTLEPEYFNSVCRLMKTIGPDMFLGTCRRCPAEIVDTVSALVYDNKLKAHKD
KSAQCFKMFYKGVITHDVSSAINRPQIGVVREFLTRNPAWRKAVFISPYNSQNAVASKILGLPTQTVD
SSQGSEYDYVIFTQTTETAHSCNVNRFNVAITRAKVGILCIMSDRDLYDKLQFTSLEIPRRNVATLQAE
NVTGLFKDCSKVITGLHPTQAPTHLSVDTKFKTEGLCVDIPGIPKDMTYRRLISMMGFKMNYQVNGY
PNMFITREEAIRHVRAWIGFDVEGCHATREAVGTNLPLQLGFSTGVNLVAVPTGYVDTPNNTDFSRVS
AKPPPGDQFKHLIPLMYKGLPWNVVRIKIVQMLSDTLKNLSDRVVFVLWAHGFELTSMKYFVKIGPE
RTCCLCDRRATCFSTASDTYACWHHSIGFDYVYNPFMIDVQQWGFTGNLQSNHDLYCQVHGNAHV
ASCDAIMTRCLAVHECFVKRVDWTIEYPIIGDELKINAACRKVQHMVVKAALLADKFPVLHDIGNPK
AIKCVPQADVEWKFYDAQPCSDKAYKIEELFYSYATHSDKFTDGVCLFWNCNVDRYPANSIVCRFDT
RVLSNLNLPGCDGGSLYVNKHAFHTPAFDKSAFVNLKQLPFFYYSDSPCESHGKQVVSDIDYVPLKS
ATCITRCNLGGAVCRHHANEYRLYLDAYNMMISAGFSLWVYKQFDTYNLWNTFTRLQSLENVAFN
GVDIAANTVIWDYKRDAPAHISTIGVCSMTDIAKKPTETICAPLTVFFDGRVDGQVDLFRNARNGVLI
TEGSVKGLQPSVGPKQASLNGVTLIGEAVKTQFNYYKKVDGVVQQLPETYFTQSRNLQEFKPRSQME
IDFLELAMDEFIERYKLEGYAFEHIVYGDFSHSQLGGLHLLIGLAKRFKESPFELEDFIPMDSTVKNYFI
TDAQTGSSKCVCSVIDLLLDDFVEIIKSQDLSVVSKVVKVTIDYTEISFMLWCKDGHVETFYPKLQSSQ
AWQPGVAMPNLYKMQRMLLEKCDLQNYGDSATLPKGIMMNVAKYTQLCQYLNTLTLAVPYNMRV
IHFGAGSDKGVAPGTAVLRQWLPTGTLLVDSDLNDFVSDADSTLIGDCATVHTANKWDLIISDMYDP
KTKNVTKENDSKEGFFTYICGFIQQKLALGGSVAIKITEHSWNADLYKLMGHFAWWTAFVTNVNASS
SEAFLIGCNYLGKPREQIDGYVMHANYIFWRNTNPIQLSSYSLFDMSKFPLKLRGTAVMSLKEGQIND
MILSLLSKGRLIIRENNRVVISSDVLVN
“SPIKE″ SARS2 Spike glycoprotein (SEQ ID NO: 70)
MFVFLVLLPLVSSQCVNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVFKNIDGYFKIYS
KHTPINLVRDLPQGFSALEPLVDLPIGINITRFQTLLALHRSYLTPGDSSSGWTAGAAAYYVGYLQPRT
FLLKYNENGTITDAVDCALDPLSETKCTLKSFTVEKGIYQTSNFRVQPTESIVRFPNITNLCPFGEVFNA
TRFASVYAWNRKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQVAVLYQDVN
CTEVPVAIHADQLTPTWRVYSTGSNVFQTRAGCLIGAEHVNNSYECDIPIGAGICASYQTQTNSPRRA
RSVASQSIIAYTMSLGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKPSKRSFIEDLLFNK
VTLADAGFIKQYGDCLGDIAARDLICAQKFNGLTVLPPLLTDEMIAQYTSALLAGTITSGWTFGAGAA
LQIPFAMQMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTAPAICHDGKAHFPREGVFV
SNGTHWFVTQRNFYEPQUITTDNTFVSGNCDVVIGIVNNTVYDPLQPELDSFKEELDKYFKNHTSPDV
DLGDISGINASVVNIQKEIDRLNEVAKNLNESLIDLQELGKYEQYIKWPWYIWLGFIAGLIAIVMVTIM
LCCMTSCCSCLKGCCSCGSCCKFDEDDSEPVLKGVKLHYT

### EQUIVALENTS

While the invention has been particularly shown and described with reference to a preferred embodiment and various alternate embodiments, it will be understood by persons skilled in the relevant art that various changes in form and details can be made therein without departing from the spirit and scope of the invention.

### INCORPORATION BY REFERENCE

All references, issued patents and patent applications cited within the body of the instant specification are hereby incorporated by reference in their entirety, for all purposes.

### REFERENCES CITED

1. Endrullat C, Glokler J, Franke P, Frohme M. (2016) Standardization and quality management in next-generation sequencing. Appl Transl Genom. 10:2-9.
2. Rice, J. J. & Daugherty, P. S. Directed evolution of a biterminal bacterial display scaffold enhances the display of diverse peptides. Protein Eng. Des. Sel. 21, 435-442 (2008).
3. Pantazes, R. J. et al. Identification of disease-specific motifs in the antibody specificity repertoire via next-generation sequencing. Sci. Rep. 6, 30312; doi: 10.1038/srep30312 (2016).
4. Dayhoff, M. & Schwartz, R. A Model of Evolutionary Change in Proteins. Atlas protein Seq. Struct. 345-352 doi: 10.1.1.145.4315 (1978).

INFORMAL SEQUENCE LISTING

| SEQ ID | | |
|---|---|---|
| NO | DESCRIPTION | SEQUENCE |
| 1 | Sequencing Adaptor Primer | TCGTCGGCAGCGTCAGATGTGTATAAG |
| | | AGACAGVBHDVCCAGTCTGGCCAGGG |
| 2 | Sequencing Adaptor Primer | GTCTCGTGGGCTCGGAGATGTGTATAA |
| | | GAGACAGGTGATGCCGTAGTACTGG |
| 3 | Peptide Motif | QPXXPFX[ED] |
| 4 | SARS-COV-2 Peptide Motif | RXX[CI]EXX[LI]F |
| 5 | SARS-COV-2 Peptide Motif | RXXQE[DN]XXF |
| 6 | SARS-COV-2 Peptide Motif | [MLIV]EXXLFXR |
| 7 | SARS-COV-2 Peptide Motif | D[ML]DXFXK |
| 8 | SARS-COV-2 Peptide Motif | FXXVC[ENA]K |
| 9 | SARS-COV-2 Peptide Motif | HGWTK |
| 10 | SARS-COV-2 Peptide Motif | [ED]XX[LM][TS][AC]XK |
| 1 | SARS-COV-2 Peptide Motif | D[PA]X[ML]X[STA]XK |
| 12 | SARS-COV-2 Peptide Motif | MT[TA][AS][KA] |
| 13 | SARS-COV-2 Peptide Motif | M[TS][ACT]AK |
| 14 | SARS-COV-2 Peptide Motif | EGXLXMXK |
| 15 | SARS-COV-2 Peptide Motif | FXXELXX[WY]F |
| 16 | SARS-COV-2 Peptide Motif | [FY]XX[QE]LXXWF |
| 17 | SARS-COV-2 Peptide Motif | QX[PT]C[LM]XK |
| 18 | SARS-COV-2 Peptide Motif | QXP[MQ]LXK |
| 19 | SARS-COV-2 Peptide Motif | QXXC[LM]XKXXF |
| 20 | SARS-COV-2 Peptide Motif | CXXXPCMXK |
| 21 | SARS-COV-2 Peptide Motif | KXQTX[ST]M |
| 22 | SARS-COV-2 Peptide Motif | QX[AS]MXPK |
| 23 | SARS-COV-2 Peptide Motif | [TS]PMXXAXXK |
| 24 | SARS-COV-2 Peptide Motif | STXXL[TS]XK |
| 25 | SARS-COV-2 Peptide Motif | QX[SN]FKXX[FL] |
| 26 | SARS-COV-2 Peptide Motif | RXPX[TS]L[ED] |
| 27 | SARS-COV-2 Peptide Motif | P[EDT]WRXY |
| 28 | SARS-COV-2 Peptide Motif | PXWR[VI]Y |
| 29 | SARS-COV-2 Peptide Motif | DXSGIXXX[DGESN] |
| 30 | SARS-COV-2 Peptide Motif | [APC]XXXDXSGI |
| 31 | SARS-COV-2 Peptide Motif | KTPP[LMI] |
| 32 | SARS-COV-2 Peptide Motif | LPFQQ |
| 33 | SARS-COV-2 Peptide Motif | K[FY][LM]XFXQ |
| 34 | SARS-COV-2 Peptide Motif | KWXXFQQ |
| 35 | SARS-COV-2 Peptide Motif | [CA]K[FY]XXFXQ |
| 36 | SARS-COV-2 Peptide Motif | CXXFIAXC |
| 37 | SARS-COV-2 Peptide Motif | CXXYWX[CA]XXK |
| 38 | SARS-COV-2 Peptide Motif | [LMI]LPXXXLD |
| 39 | SARS-COV-2 Peptide Motif | KXXGPXXLY |
| 40 | SARS-COV-2 Peptide Motif | QGPT[LI] |
| 41 | SARS-COV-2 Peptide Motif | KXG[TCA]PXXXXP |
| 42 | SARS-COV-2 Peptide Motif | WX[RK]A[DV]S |
| 43 | SARS-COV-2 Peptide Motif | [WTHN]XXXRA\|VA]S |
| 44 | SARS-COV-2 Peptide Motif | [FY][RK]A[DAV][TS] |
| 45 | SARS-COV-2 Peptide Motif | CXXC[KR]AXS |
| 46 | SARS-COV-2 Peptide Motif | LX[AP]R[RKA]D |
| 47 | SARS-COV-2 Peptide Motif | FX[RK][RK]DT |
| 48 | SARS-COV-2 Peptide Motif | [RK][RKA]D[CTS]N |
| 49 | SARS-COV-2 Peptide Motif | [YF][RK]R[DA]XXX[PVR] |
| 50 | SARS-COV-2 Peptide Motif | WX[RK]MAD |
| 51 | SARS-COV-2 Peptide Motif | RMADXXR |
| 52 | SARS-COV-2 Peptide Motif | PQXXXDK[TSR] |
| 53 | SARS-COV-2 Peptide Motif | [IV]XXX[QR][DN]KXH |
| 54 | SARS-COV-2 Peptide Motif | D[STR]NG[TS] |
| 55 | SARS-COV-2 Peptide Motif | D[SKH]NGT |
| 56 | SARS-COV-2 Peptide Motif | [VI]DXXG[TS] [IL] |
| 57 | SARS-COV-2 Peptide Motif | DXXG[TS][FWY][MV] |
| 58 | SARS-COV-2 Peptide Motif | [AI]DXXGTX[CHT] |
| 59 | SARS-COV-2 Peptide Motif | CXXDXXGIST]C |
| 60 | SARS-COV-2 Peptide Motif | DXNX[TS]XK[YF] |
| 61 | SARS-COV-2 Peptide Motif | PNG[TS]XXP |
| 62 | SARS-COV-2 Peptide Motif | GXVDWD |
| 63 | SARS-COV-2 Peptide Motif | CXPXCXEXXW |
| 64 | SARS-COV-2 Peptide Motif | [AT]RA[VL]X[NTE] |

1. (canceled)

2. A peptide display system, wherein the display system comprises:
(a) one or more display surfaces; and
(b) at least one distinct peptide, wherein the at least one distinct peptides comprises a binding motif, wherein the binding motif is selected from a motif group consisting of:
(SEQ ID NO: 73)
[AC]K[FY]XXFXQ,
(SEQ ID NO: 96)
[ACP]XXXDXSGI,
(SEQ ID NO: 58)
[AI]DXXGTX[CHT],
(SEQ ID NO: 190)
[AIV]XXNG[ST]X[FLW],
(SEQ ID NO: 174)
[AP][KR]AXSX[DENS],
(SEQ ID NO: 97)
[AP]Q[EG]XTXXK,
(SEQ ID NO: 159)
[AS][KR]MAXS,
(SEQ ID NO: 134)
[AS]AX[FY]MXK,
(SEQ ID NO: 64)
[AT]RA[VL]X[NTE],
(SEQ ID NO: 170)
[AT]XAVSXXX[HNY],
(SEQ ID NO: 109)
[DE]CX[AGM]XKXC,
(SEQ ID NO: 119)
[DE]GXLX[LM]XK,
(SEQ ID NO: 122)
[DE]PX[LM]X[ACS]XK,
(SEQ ID NO: 201)
[DN]AAXAL,
(SEQ ID NO: 10)
[ED]XX[LM][TS][AC]XK,
(SEQ ID NO: 84)
[EFY]TX[PS]LG,
(SEQ ID NO: 200)
[FI]AXEXDK,
(SEQ ID NO: 113)
[FL][DN]XY[FY]XD,
(SEQ ID NO: 160)
[FW][FY]XAXS[FY],
(SEQ ID NO: 182)
[FW]XRRDXG,
(SEQ ID NO: 185)
[FY][KR][KR]DXXXP,
(SEQ ID NO: 44)
[FY][RK]A[DAV][TS],
(SEQ ID NO: 105)
[FY]PXTXPK),
(SEQ ID NO: 102)
[FY]X[LV]LXXLN,
(SEQ ID NO: 178)
[FY]XKAXSXXY,
(SEQ ID NO: 180)
[FY]XRAMS,
(SEQ ID NO: 16)
[FY]XX[QE]LXXWF,
(SEQ ID NO: 202)
[GS][AS]AXXX[KR]K,
(SEQ ID NO: 115)
[GST]PXQYXD,
(SEQ ID NO: 171)
[HQ]X[AST][KR]AXS,
(SEQ ID NO: 107)
[ILM]LPXXXLD,
(SEQ ID NO: 92)
[ILMV]EXXLFXR,
(SEQ ID NO: 177)
[IMN][LM]X[KR]AXS,
(SEQ ID NO: 168)
[IR][FNW][KR]AXS,
(SEQ ID NO: 81)
[ITV]P[ST]WR,
(SEQ ID NO: 191)
[ITV]XXNG[ST][ILV],
(SEQ ID NO: 53)
[IV]XXX[QR][DN]KXH,
(SEQ ID NO: 187)
[KR][AKR]D[CST]N,
(SEQ ID NO: 186)
[KR][KR]DCXXXC,
(SEQ ID NO: 161)
[KR]AVS[DEGNQ],
(SEQ ID NO: 165)
[KR]AVXX[GR]X[FY],
(SEQ ID NO: 175)
[KR]AXSMXXR,
(SEQ ID NO: 179)
[KR]AXSX[LM][DE],
(SEQ ID NO: 167)
[KR]AXSXX[RW]M,
(SEQ ID NO: 166)
[KR]AXSXX[ST]X[HRS],
(SEQ ID NO: 176)
[KR]AXSXXX[HP][HY],
(SEQ ID NO: 103)
[LM]X[HR]LNX[ILM],
(SEQ ID NO: 131)
[NST]P[FM]XXX[ST]XK,
(SEQ ID NO: 48)
[RK][RKA]D[CTS]N,
(SEQ ID NO: 199)
[RK]S[ML]AS,
(SEQ ID NO: 189)
[RY]ADXNG,
(SEQ ID NO: 132)
[ST]PMXXAXXK,
(SEQ ID NO: 56)
[VI]DXXG[TS][IL],
(SEQ ID NO: 43)
[WTHN]XXXRA[VA]S,
(SEQ ID NO: 49)
[YF][RK]R[DA]XXX[PVR],
(SEQ ID NO: 197)
A[FIY]SG[GHS],
(SEQ ID NO: 163)
AVSX[MNQ][HL],
(SEQ ID NO: 149)
AXX[MS]RKP,
(SEQ ID NO: 196)
CSTIXC,
(SEQ ID NO: 147)
CXEX[ILM]LF,
(SEQ ID NO: 142)
CXLXWSXC,
(SEQ ID NO: 153)
CXVXPXMXK,
(SEQ ID NO: 45)
CXXC[KR]AXS,
(SEQ ID NO: 59)
CXXDXXG[ST]C,
(SEQ ID NO: 36)
CXXFIAXC,
(SEQ ID NO: 20)
CXXXPCMXK,
(SEQ ID NO: 116)
CXXXXCVMXK,
(SEQ ID NO: 152)
CXXYWX[AC]XXK,
(SEQ ID NO: 108)
D[LM]DXFXK,
(SEQ ID NO: 95)
D[LMY]SGI,
(SEQ ID NO: 192)
D[MR]NX[ST][LW],
(SEQ ID NO: 11)
D[PA]X[ML]X[STA]XK,
(SEQ ID NO: 55)
D[SKH]NGT,
(SEQ ID NO: 54)
D[STR]NG[TS],
(SEQ ID NO: 188)
DSNG[ST],
(SEQ ID NO: 110)
DX[AC][GM][AFS]K,
(SEQ ID NO: 111)
DXAGXK,
(SEQ ID NO: 193)
DXNX[ST][CY][TW],
(SEQ ID NO: 60)
DXNX[TS]XK[YF],
(SEQ ID NO: 29)
DXSGIXXX[DGESN],
(SEQ ID NO: 57)
DXXG[TS][FWY][MV],
(SEQ ID NO: 14)
EGXLXMXK,
(SEQ ID NO: 85)
ETXS[LM]G,
(SEQ ID NO: 104)
FP[AP][MT]XXK,
(SEQ ID NO: 47)
FX[RK][RK]DT,
(SEQ ID NO: 146)
FXE[HR]XK[PR],
(SEQ ID NO: 118)
FXQVCXK,
(SEQ ID NO: 184)
FXR[AK]DXXXP,
(SEQ ID NO: 15)
FXXELXX[WY]F,
(SEQ ID NO: 117)
FXXV[MT]RK,
(SEQ ID NO: 8)
FXXVC[ENA]K,
(SEQ ID NO: 9)
HGWTK,
(SEQ ID NO: 99)
HXQCPXXXK,
(SEQ ID NO: 195)
IPGS[FY],
(SEQ ID NO: 172)
IXXAVSX[AS],
(SEQ ID NO: 140)
K[DE]X[IV]PXXD,
(SEQ ID NO: 203)
K[DS]G[ILV]XW,
(SEQ ID NO: 33)
K[FY][LM]XFXQ,
(SEQ ID NO: 72)
K[WY]X[AP]FXQ,
(SEQ ID NO: 169)
KAXSXXXXXQ,
(SEQ ID NO: 173)
KAXSXXXY,
(SEQ ID NO: 93)
KEXLXXWF,
(SEQ ID NO: 141)
KMXPWXQ,
(SEQ ID NO: 86)
KTPP[ILM],
(SEQ ID NO: 34)
KWXXFQQ,
(SEQ ID NO: 150)
KXG[ACT]PXXXXP,
(SEQ ID NO: 155)
KXP[FW]CP,
(SEQ ID NO: 21)
KXQTX[ST]M,
(SEQ ID NO: 91)
KXSXIEX[ILM],
(SEQ ID NO: 71)
KXX[AG]XQQ[FY],
(SEQ ID NO: 114)
KXX[IV]Q[FY]XD,
(SEQ ID NO: 39)
KXXGPXXLY,
(SEQ ID NO: 139)
KXXIPYXD,
(SEQ ID NO: 106)
LLPXXX[IL]D,
(SEQ ID NO: 32)
LPFQQ,
(SEQ ID NO: 46)
LX[AP]R[RKA]D,
(SEQ ID NO: 101)
LX[IL]LXXLN,
(SEQ ID NO: 123)
M[ST][ACT]AK,
(SEQ ID NO: 124)
MT[AT][AS][AK],
(SEQ ID NO: 27)
P[EDT]WRXY,
(SEQ ID NO: 61)
PNG[TS]XXP,
(SEQ ID NO: 52)
PQXXXDK[TSR],
(SEQ ID NO: 194)
PX[ANP][QR]XDK,
(SEQ ID NO: 79)
PXWR[IV]Y,
(SEQ ID NO: 148)
PXWXXG[LM]D,
(SEQ ID NO: 162)
PXXRAXSXX[HPW],
(SEQ ID NO: 164)
PXXXRAXSXG,
(SEQ ID NO: 40)
QGPT[LI],
(SEQ ID NO: 98)
QGPTXP,
(SEQ ID NO: 22)
QX[AS]MXPK,
(SEQ ID NO: 137)
QX[NS]FKXX[FL],
(SEQ ID NO: 17)
QX[PT]C[LM]XK,
(SEQ ID NO: 18)
QXP[MQ]LXK,
(SEQ ID NO: 136)
QXSFKXX[FMW],
(SEQ ID NO: 19)
QXXC[LM]XKXXF,
(SEQ ID NO: 87)
R[EHS]XXEXXLF,
(SEQ ID NO: 145)
R[FH]PEK,
(SEQ ID NO: 198)
RD[ST]XXPXR,
(SEQ ID NO: 157)
RMADX[AHR],
(SEQ ID NO: 51)
RMADXXR,
(SEQ ID NO: 82)
RS[ILMV]AS,
(SEQ ID NO: 77)
RXPX[ST]L[DE],
(SEQ ID NO: 78)
RXPX[ST]LE,
(SEQ ID NO: 89)
RXX[CI]EXX[IL]F,
(SEQ ID NO: 5)
RXXQE[DN]XXF,
(SEQ ID NO: 88)
RXXQEXXLF,
(SEQ ID NO: 135)
SAXY[AMS]XK,
(SEQ ID NO: 130)
STXXL[ST]XK,
(SEQ ID NO: 90)
SXXEDXLF,
(SEQ ID NO: 129)
TPMXXX[ST]XK,
(SEQ ID NO: 158)
WAXMAD,
(SEQ ID NO: 144)
WF[DE]XID,
(SEQ ID NO: 120)
WWXXL[LMT]XK,
(SEQ ID NO: 183)
WX[KR][AKR]D[ST],
(SEQ ID NO: 42)
WX[RK]A[DV]S,
(SEQ ID NO: 50)
WX[RK]MAD,
(SEQ ID NO: 121)
WXX[IL]M[AL]K,
(SEQ ID NO: 181)
Y[KR]XD[ST][NY],
(SEQ ID NO: 154)
YCXAXKXC,
(SEQ ID NO: 151)
YWXYFXK,
(SEQ ID NO: 143)
YX[IR][FY]ML,
and wherein the at least one distinct peptide extends from the one or more display surfaces.

3. The display system of claim 2, wherein the display system comprises:
(a) one or more display surfaces; and
(b) at least one distinct peptide, wherein the at least one distinct peptides comprises a binding motif, wherein the binding motif is selected from a motif group consisting of:
(i)
(SEQ ID NO: 4)
RXX[CI]EXX[LI]F,
(SEQ ID NO: 5)
RXXQE[DN]XXF,
(SEQ ID NO: 6)
[MLIV]EXXLFXR,
(SEQ ID NO: 7)
D[ML]DXFXK,
(SEQ ID NO: 8)
FXXVC[ENA]K,
(SEQ ID NO: 9)
HGWTK,
(SEQ ID NO: 10)
[ED]XX[LM][TS][AC]XK,
(SEQ ID NO: 11)
D[PA]X[ML]X[STA]XK,
(SEQ ID NO: 12)
MT[TATAS][KA],
(SEQ ID NO: 13)
M[TS][ACT]AK,
(SEQ ID NO: 14)
EGXLXMXK,
(SEQ ID NO: 15)
FXXELXX[WY]F,
(SEQ ID NO: 16)
[FY]XX[QE]LXXWF,
(SEQ ID NO: 17)
QX[PT]C[LM]XK,
(SEQ ID NO: 18)
QXP[MQ]LXK,
(SEQ ID NO: 19)
QXXC[LM]XKXXF,
(SEQ ID NO: 20)
CXXXPCMXK,
(SEQ ID NO: 21)
KXQTX[ST]M,
(SEQ ID NO: 22)
QX[AS]MXPK,
(SEQ ID NO: 23)
[TS]PMXXAXXK,
(SEQ ID NO: 24)
STXXL[TS]XK,
(SEQ ID NO: 25)
QX[SN]FKXX[FL],
(SEQ ID NO: 26)
RXPX[TS]L[ED],
(SEQ ID NO: 27)
P[EDT]WRXY,
(SEQ ID NO: 28)
PXWR[VI]Y,
(SEQ ID NO: 29)
DXSGIXXX[DGESN],
(SEQ ID NO: 30)
[APC]XXXDXSGI,
(SEQ ID NO: 31)
KTPP[LMI],
(SEQ ID NO: 32)
LPFQQ,
(SEQ ID NO: 33)
K[FY][LM]XFXQ,
(SEQ ID NO: 34)
KWXXFQQ,
(SEQ ID NO: 35)
[CA]K[FY]XXFXQ,
(SEQ ID NO: 36)
CXXFIAXC,
(SEQ ID NO: 37)
CXXYWX[CA]XXK,
(SEQ ID NO: 38)
[LMI]LPXXXLD,
(SEQ ID NO: 39)
KXXGPXXLY,
(SEQ ID NO: 40)
QGPT[LI],
(SEQ ID NO: 41)
KXG[TCA]PXXXXP,
(SEQ ID NO: 42)
WX[RK]A[DV]S,
(SEQ ID NO: 43)
[WTHN]XXXRA[VA]S,
(SEQ ID NO: 44)
[FY][RK]A[DAV][TS],
(SEQ ID NO: 45)
CXXC[KR]AXS,
(SEQ ID NO: 46)
LX[AP]R[RKA]D,
(SEQ ID NO: 47)
FX[RK][RK]DT,
(SEQ ID NO: 48)
[RK][RKA]D[CTS]N,
(SEQ ID NO: 49)
[YFIRK]R[DA]XXX[PVR],
(SEQ ID NO: 50)
WX[RK]MAD,
(SEQ ID NO: 51)
RMADXXR,
(SEQ ID NO: 52)
PQXXXDK[TSR],
(SEQ ID NO: 53)
[IV]XXX[QR][DN]KXH,
(SEQ ID NO: 54)
D[STRING[TS],
(SEQ ID NO: 55)
D[SKH]NGT,
(SEQ ID NO: 56)
[VI]DXXG[TS][IL],
(SEQ ID NO: 57)
DXXG[TS][FWY][MV],
(SEQ ID NO: 58)
[AI]DXXGTX[CHT],
(SEQ ID NO: 59)
CXXDXXGISTIC,
(SEQ ID NO: 60)
DXNX[TS]XK[YF],
(SEQ ID NO: 61)
PNG[TS]XXP,
(SEQ ID NO: 62)
GXVDWD,
(SEQ ID NO: 63)
CXPXCXEXXW,
(SEQ ID NO: 64)
[AT]RA[VL]X[NTE];
or
(ii)
(SEQ ID NO: 97)
[AP]Q[EG]XTXXK,
(SEQ ID NO: 134)
[AS]AX[FY]MXK,
(SEQ ID NO: 109)
[DE]CX[AGM]XKXC,
(SEQ ID NO: 122)
[DE]PX[LM]X[ACS]XK,
(SEQ ID NO: 84)
[EFY]TX[PS]LG,
(SEQ ID NO: 113)
[FL][DN]XY[FY]XD,
(SEQ ID NO: 153)
CXVXPXMXK,
(SEQ ID NO: 116)
CXXXXCVMXK,
(SEQ ID NO: 95)
D[LMY]SGI,
(SEQ ID NO: 14)
EGXLXMXK,
(SEQ ID NO: 104)
FP[AP][MT]XXK,
(SEQ ID NO: 15)
FXXELXX[WY]F,
(SEQ ID NO: 71)
KXX[AG]XQQ[FY],
(SEQ ID NO: 139)
KXXIPYXD,
(SEQ ID NO: 106)
LLPXXX[IL]D,
(SEQ ID NO: 101)
LX[IL]LXXLN,
(SEQ ID NO: 79)
PXWR[IV]Y,
(SEQ ID NO: 136)
QXSFKXX[FMW],
(SEQ ID NO: 87)
R[EHS]XXEXXLF,
(SEQ ID NO: 82)
RS[ILMV]AS,
(SEQ ID NO: 77)
RXPX[ST]L[DE],
(SEQ ID NO: 129)
TPMXXX[ST]XK,
(SEQ ID NO: 120)
WWXXL[LMT]XK,
(SEQ ID NO: 73)
[AC]K[FY]XXFXQ,
(SEQ ID NO: 96)
[ACP]XXXDXSGI,
(SEQ ID NO: 119)
[DE]GXLX[LM]XK,
(SEQ ID NO: 201)
[DN]AAXAL,
(SEQ ID NO: 10)
[ED]XX[LM][TS][AC]XK,
(SEQ ID NO: 200)
[FI]AXEXDK,
(SEQ ID NO: 105)
[FY]PXTXPK,
(SEQ ID NO: 102)
[FY]X[LV]LXXLN,
(SEQ ID NO: 16)
[FY]XX[QE]LXXWF,
(SEQ ID NO: 202)
[GS][AS]AXXX[KR]K,
(SEQ ID NO: 115)
[GST]PXQYXD,
(SEQ IDNO: 107)
[ILM]LPXXXLD,
(SEQ ID NO: 92)
[ILMV]EXXLFXR,
(SEQ ID NO: 81)
[ITV]P[ST]WR,
(SEQ ID NO: 103)
[LM]X[HR]LNX[ILM],
(SEQ ID NO: 131)
[NST]P[FM]XXX[ST]XK,
(SEQ ID NO: 199)
[RK]S[ML]AS,
(SEQ ID NO: 132)
[ST]PMXXAXXK,
(SEQ ID NO: 149)
AXX[MS]RKP,
(SEQ ID NO: 147)
CXEX[ILM]LF,
(SEQ ID NO: 142)
CXLXWSXC,
(SEQ ID NO: 36)
CXXFIAXC,
(SEQ ID NO: 20)
CXXXPCMXK,
(SEQ ID NO: 152)
CXXYWX[AC]XXK,
(SEQ ID NO: 108)
D[LM]DXFXK,
(SEQ ID NO: 11)
D[PA]X[ML]X[STA]XK,
(SEQ ID NO: 110)
DX[AC][GM][AFS]K,
(SEQ ID NO: 111)
DXAGXK,
(SEQ ID NO: 29)
DXSGIXXX[DGESN],
(SEQ ID NO: 85)
ETXS[LM]G,
(SEQ ID NO: 146)
FXE[HR]XK[PR],
(SEQ ID NO: 118)
FXQVCXK,
(SEQ ID NO: 117)
FXXV[MT]RK,
(SEQ ID NO: 8)
FXXVC[ENA]K,
(SEQ ID NO: 9)
HGWTK,
(SEQ ID NO: 99)
HXQCPXXXK,
(SEQ ID NO: 140)
K[DE]X[IV]PXXD,
(SEQ ID NO: 203)
K[DS]G[ILV]XW,
(SEQ ID NO: 33)
K[FY][LM]XFXQ,
(SEQ ID NO: 72)
K[WY]X[AP]FXQ,
(SEQ ID NO: 93)
KEXLXXWF,
(SEQ ID NO: 141)
KMXPWXQ,
(SEQ ID NO: 86)
KTPP[ILM],
(SEQ ID NO: 34)
KWXXFQQ,
(SEQ ID NO: 150)
KXG[ACT]PXXXXP,
(SEQ ID NO: 155)
KXP[FW]CP,
(SEQ ID NO: 21)
KXQTX[ST]M,
(SEQ ID NO: 91)
KXSXIEX[ILM],
(SEQ ID NO: 114)
KXX[IV]Q[FY]XD,
(SEQ ID NO: 39)
KXXGPXXLY,
(SEQ ID NO: 32)
LPFQQ,
(SEQ ID NO: 123)
M[ST][ACT]AK,
(SEQ ID NO: 124)
MT[AT][AS][AK],
(SEQ ID NO: 27)
P[EDT]WRXY,
(SEQ ID NO: 148)
PXWXXG[LM]D,
(SEQ ID NO: 40)
QGPT[LI],
(SEQ ID NO: 98)
QGPTXP,
(SEQ ID NO: 22)
QX[AS]MXPK,
(SEQ ID NO: 137)
QX[NS]FKXX[FL],
(SEQ ID NO: 17)
QX[PT]C[LM]XK,
(SEQ ID NO: 18)
QXP[MQ]LXK,
(SEQ ID NO: 19)
QXXC[LM]XKXXF,
(SEQ ID NO: 145)
R[FH]PEK,
(SEQ ID NO: 78)
RXPX[ST]LE,
(SEQ ID NO: 89)
RXX[CI]EXX[IL]F,
(SEQ ID NO: 5)
RXXQE[DN]XXF,
(SEQ ID NO: 88)
RXXQEXXLF,
(SEQ ID NO: 135)
SAXY[AMS]XK,
(SEQ ID NO: 130)
STXXL[ST]XK,
(SEQ ID NO: 90)
SXXEDXLF,
(SEQ ID NO: 144)
WF[DE]XID,
(SEQ ID NO: 121)
WXX[IL]M[AL]K,
(SEQ ID NO: 154)
YCXAXKXC,
(SEQ ID NO: 151)
YWXYFXK,
(SEQ ID NO: 143)
YX[IR][FY]ML
(iii)
(SEQ ID NO: 79)
PXWR[IV]Y,
(SEQ ID NO: 27)
P[EDT]WRXY,
(SEQ ID NO: 81)
[ITV]P[ST]WR,
(SEQ ID NO: 84)
[EFY]TX[PS]LG,
(SEQ ID NO: 85)
ETXS[LM]G,
(SEQ ID NO: 86)
KTPP[ILM],
(SEQ ID NO: 95)
D[LMY]SGI,
(SEQ ID NO: 96)
[ACP]XXXDXSGI,
(SEQ ID NO: 203)
K[DS]G[ILV]XW,
(SEQ ID NO: 97)
[AP]Q[EG]XTXXK,
(SEQ ID NO: 98)
QGPTXP,
(SEQ ID NO: 99)
HXQCPXXXK,
(SEQ ID NO: 18)
QXP[MQ]LXK,
(SEQ ID NO: 101)
LX[IL]LXXLN,
(SEQ ID NO: 102)
[FY]X[LV]LXXLN,
(SEQ ID NO: 202)
[GSTAS]AXXX[KR]K,
(SEQ ID NO: 104)
FP[AP][MT]XXK,
(SEQ ID NO: 105)
[FY]PXTXPK,
(SEQ ID NO: 21)
KXQTX[ST]M,
(SEQ ID NO: 107)
[ILM]LPXXXLD,
(SEQ ID NO: 113)
[FL][DN]XY[FY]XD,
(SEQ ID NO: 114)
KXX[IV]Q[FY]XD,
(SEQ ID NO: 117)
FXXV[MT]RK,
(SEQ ID NO: 118)
FXQVCXK,
(SEQ ID NO: 8)
FXXVC[ENA]K,
(SEQ ID NO: 14)
EGXLXMXK,
(SEQ ID NO: 119)
[DE]GXLX[LM]XK,
(SEQ ID NO: 120)
WWXXL[LMT]XK,
(SEQ ID NO: 121)
WXX[IL]M[AL]K,
(SEQ ID NO: 122)
[DE]PX[LM]X[ACS]XK,
(SEQ ID NO: 123)
M[ST][ACT]AK,
(SEQ ID NO: 124)
MT[AT][AS][AK],
(SEQ ID NO: 10)
[ED]XX[LM][TS][AC]XK,
(SEQ ID NO: 11)
D[PA]X[ML]X[STA]XK,
(SEQ ID NO: 129)
TPMXXX[ST]XK,
(SEQ ID NO: 130)
STXXL[ST]XK,
(SEQ ID NO: 131)
[NST]P[FM]XXX[ST]XK,
(SEQ ID NO: 132)
[ST]PMXXAXXK,
(SEQ ID NO: 134)
[AS]AX[FY]MXK,
(SEQ ID NO: 135)
SAXY[AMS]XK,
(SEQ ID NO: 136)
QXSFKXX[FMW],
(SEQ ID NO: 137)
QX[NS]FKXX[FL],
(SEQ ID NO: 140)
K[DE]X[IV]PXXD,
(SEQ ID NO: 141)
KMXPWXQ,
(SEQ ID NO: 142)
CXLXWSXC,
(SEQ ID NO: 143)
YX[IR][FY]ML,
(SEQ ID NO: 144)
WF[DE]XID,
(SEQ ID NO: 145)
R[FH]PEK,
(SEQ ID NO: 146)
FXE[HR]XK[PR],
(SEQ ID NO: 148)
PXWXXG[LM]D,
(SEQ ID NO: 149)
AXX[MS]RKP,
(SEQ ID NO: 36)
CXXFIAXC,
(SEQ ID NO: 9)
HGWTK,
(SEQ ID NO: 150)
KXG[ACT]PXXXXP,
(SEQ ID NO: 151)
YWXYFXK,
(SEQ ID NO: 152)
CXXYWX[AC]XXK,
(SEQ ID NO: 153)
CXVXPXMXK,
(SEQ ID NO: 20)
CXXXPCMXK,
(SEQ ID NO: 154)
YCXAXKXC,
(SEQ ID NO: 39)
KXXGPXXLY;
or
(iv)
(SEQ ID NO: 134)
[AS]AX[FY]MXK,
(SEQ ID NO: 122)
[DE]PX[LM]X[ACS]XK,
(SEQ ID NO: 9)
HGWTK,
(SEQ ID NO: 145)
R[FH]PEK,
(SEQ ID NO: 97)
[AP]Q[EG]XTXXK,
(SEQ ID NO: 113)
[FL][DN]XY[FY]XD,
(SEQ ID NO: 149)
AXX[MS]RKP,
(SEQ ID NO: 108)
D[LM]DXFXK,
(SEQ ID NO: 14)
EGXLXMXK,
(SEQ ID NO: 21)
KXQTX[ST]M,
(SEQ ID NO: 39)
KXXGPXXLY,
(SEQ ID NO: 101)
LX[IL]LXXLN,
(SEQ ID NO: 195)
IPGS[FY],
(SEQ ID NO: 197)
A[FIY]SG[GHS],
and
wherein the at least one distinct peptide extends from the one or more display surfaces.

4. (canceled)

5. (canceled)

6. The display system of claim 2, wherein the display system comprises at least 2 distinct peptides each comprising a distinct binding motif, wherein each distinct binding motif is selected from the motif group.

7. The display system of claim 2, wherein the display system comprises at least 3, at least 4, at least 5, at least 6, at least 7, at least 8, at least 9, at least 10, at least 15, at least 20, at least 25, at least 30, at least 35, at least 40, at least 45, at least 50, at least 55, or at least 60 distinct peptides each comprising a distinct binding motif, wherein each distinct binding motif is selected from the motif group.

8. (canceled)

9. (canceled)

10. The display system of claim 2, wherein the at least one distinct peptide is capable of binding to an antibody associated with a SARS-CoV-2 infection.

11. The display system of claim 2, wherein the one or more display surfaces comprises a single display surface.

12. (canceled)

13. (canceled)

14. The display system of claim 11, wherein the single display surface is a peptide microarray, a multi-well plate, or a lateral flow assay.

15. (canceled)

16. (canceled)

17. (canceled)

18. (canceled)

19. (canceled)

20. The display system of claim 2, wherein the one or more display surfaces comprises a plurality of display surfaces.

21. (canceled)

22. (canceled)

23. The display system of claim 21, wherein the at least one distinct peptide is encoded by one or more distinct nucleic acid sequences capable of expressing the at least one distinct peptide in the plurality of biological entities.

24. (canceled)

25. (canceled)

26. (canceled)

27. The display system of claim 20, wherein the plurality of display surfaces is a plurality of microparticle surfaces.

28. The display system of claim 20, wherein each of the plurality of display surfaces comprises a distinct single species of the at least one distinct peptides.

29. The display system of claim 2, wherein the at least one distinct peptide further comprises a label.

30. The display system of any of claim 2, wherein the one or more display surfaces or the distinct peptides is bound or capable of being bound directly or indirectly to a capture entity.

31. The display system of claim 30, wherein the capture entity comprises:
a) a solid support selected from the group consisting of: an agarose bead, a sepharose bead, a magnetic bead, and a resin; and
(b) either (i) a binding moiety that binds directly to the one or more display surfaces or the distinct peptides or (ii) a binding moiety that binds a binding molecule that binds to the one or more display surfaces or at least one of the distinct peptides.

32. (canceled)

33. (canceled)

34. The display system of claim 33, wherein:
(a) the binding molecule that binds to at least one of the distinct peptides is an antibody from a biological sample; or
(b) the binding moiety is selected from the group consisting of: Protein A, Protein G, Protein A/G, and an antibody or antigen binding fragment thereof; or
(c) the binding moiety comprises streptavidin and the one or more display surfaces, the distinct peptide, or the binding molecule that binds to the one or more display surfaces or at least one of the distinct peptides further comprises biotin.

35. (canceled)

36. (canceled)

37. (canceled)

38. (canceled)

39. (canceled)

40. (canceled)

41. (canceled)

42. The display system of f m claim 2, wherein the composition further comprises a library of peptides.

43. (canceled)

44. The display system of claim 42, wherein the library of peptides is a non-random peptide library.

45. The display system of claim 44, wherein the non-random library is biased to represent one or more diseases or conditions, wherein the biased representation is at least greater than a representation in a random peptide library, and wherein at least one of the one or more diseases or conditions is a SARS-CoV-2 infection.

46. (canceled)

47. (canceled)

48. (canceled)

49. (canceled)

50. (canceled)

51. (canceled)

52. (canceled)

53. (canceled)

54. (canceled)

55. The display system of any one of claims 1-54, wherein the binding motifs are selected from IgG isotype specific binding motifs, IgM isotype specific binding motifs, or a combination of IgG and IgM isotype specific binding motifs.

56. (canceled)

57. A method of determining the presence of specimen antibodies specific for SARS-CoV-2 in a biological sample comprising:
contacting the display system of claim 2 with a biological sample, wherein the biological sample comprises a plurality of antibodies, wherein the plurality of antibodies is known or suspected to comprise specimen antibodies specific for SARS-CoV-2, and wherein the contacting is under conditions sufficient for the specimen antibodies specific for SARS-CoV-2 to bind a cognate epitope;
measuring the binding between the at least one distinct peptide and the specimen antibodies; and
determining specimen antibodies specific for SARS-CoV-2 are present in the biological sample when binding between at least one of the at least one distinct peptides and the specimen antibodies is detected.

58-106. (canceled)

107. A peptide expression library, wherein the library comprises a library of nucleic acid sequences encoding a library of peptides, wherein the library of peptides comprises at least one distinct peptide, wherein the at least one distinct peptide comprises a binding motif, wherein the binding motif is selected from a motif group consisting of:
(SEQ ID NO: 73)
[AC]K[FY]XXFXQ,
(SEQ ID NO: 96)
[ACP]XXXDXSGI,
(SEQ ID NO: 58)
[AI]DXXGTX[CHT],
(SEQ ID NO: 190)
[AIV]XXNG[ST]X[FLW],
(SEQ ID NO: 174)
[AP][KR]AXSX[DENS],
(SEQ ID NO: 97)
[AP]Q[EG]XTXXK,
(SEQ ID NO: 159)
[AS][KR]MAXS,
(SEQ ID NO: 134)
[AS]AX[FY]MXK,
(SEQ ID NO: 64)
[AT]RA[VL]X[NTE],
(SEQ ID NO: 170)
[AT]XAVSXXX[HNY],
(SEQ ID NO: 109)
[DE]CX[AGM]XKXC,
(SEQ ID NO: 119)
[DE]GXLX[LM]XK,
(SEQ ID NO: 122)
[DE]PX[LM]X[ACS]XK,
(SEQ ID NO: 201)
[DN]AAXAL,
(SEQ ID NO: 10)
[ED]XX[LM][TS][AC]XK,
(SEQ ID NO: 84)
[EFY]TX[PS]LG,
(SEQ ID NO: 200)
[FI]AXEXDK,
(SEQ ID NO: 113)
[FL][DN]XY[FY]XD,
(SEQ ID NO: 160)
[FW][FY]XAXS[FY],
(SEQ ID NO: 182)
[FW]XRRDXG,
(SEQ ID NO: 185)
[FY][KR][KR]DXXXP,
(SEQ ID NO: 44)
[FY][RK]A[DAV][TS],
(SEQ ID NO: 105)
[FY]PXTXPK,
(SEQ ID NO: 102)
[FY]X[LV]LXXLN,
(SEQ ID NO: 178)
[FY]XKAXSXXY,
(SEQ ID NO: 180)
[FY]XRAMS,
(SEQ ID NO: 16)
[FY]XX[QE]LXXWF,
(SEQ ID NO: 202)
[GS][AS]AXXX[KR]K,
(SEQ ID NO: 115)
[GST]PXQYXD,
(SEQ ID NO: 171)
[HQ]X[AST][KR]AXS,
(SEQ ID NO: 107)
[ILM]LPXXXLD,
(SEQ ID NO: 92)
[ILMV]EXXLFXR,
(SEQ ID NO: 177)
[IMN][LM]X[KR]AXS,
(SEQ ID NO: 168)
[IR][FNW][KR]AXS,
(SEQ ID NO: 81)
[ITV]P[ST]WR,
(SEQ ID NO: 191)
[ITV]XXNG[ST][ILV],
(SEQ ID NO: 53)
[IV]XXX[QR][DN]KXH,
(SEQ ID NO: 187)
[KR][AKR]D[CST]N,
(SEQ ID NO: 186)
[KR][KR]DCXXXC,
(SEQ ID NO: 161)
[KR]AVS[DEGNQ],
(SEQ ID NO: 165)
[KR]AVXX[GR]X[FY],
(SEQ ID NO: 175)
[KR]AXSMXXR,
(SEQ ID NO: 179)
[KR]AXSX[LM][DE],
(SEQ ID NO: 167)
[KR]AXSXX[RW]M,
(SEQ ID NO: 166)
[KR]AXSXX[ST]X[HRS],
(SEQ ID NO: 176)
[KR]AXSXXX[HP][HY],
(SEQ ID NO: 103)
[LM]X[HR]LNX[ILM],
(SEQ ID NO: 131)
[NST]P[FM]XXX[ST]XK,
(SEQ ID NO: 48)
[RK][RKA]D[CTS]N,
(SEQ ID NO: 199)
[RK]S[ML]AS,
(SEQ ID NO: 189)
[RY]ADXNG,
(SEQ ID NO: 132)
[ST]PMXXAXXK,
(SEQ ID NO: 56)
[VI]DXXG[TS][IL],
(SEQ ID NO: 43)
[WTHN]XXXRA[VA]S,
(SEQ ID NO: 49)
[YF][RK]R[DA]XXX[PVR],
(SEQ ID NO: 197)
A[FIY]SG[GHS],
(SEQ ID NO: 163)
AVSX[MNQ][HL],
(SEQ ID NO: 149)
AXX[MS]RKP,
(SEQ ID NO: 196)
CSTIXC,
(SEQ ID NO: 147)
CXEX[ILM]LF,
(SEQ ID NO: 142)
CXLXWSXC,
(SEQ ID NO: 153)
CXVXPXMXK,
(SEQ ID NO: 45)
CXXC[KR]AXS,
(SEQ ID NO: 59)
CXXDXXG[ST]C,
(SEQ ID NO: 36)
CXXFIAXC,
(SEQ ID NO: 20)
CXXXPCMXK,
(SEQ ID NO: 116)
CXXXXCVMXK,
(SEQ ID NO: 152)
CXXYWX[AC]XXK,
(SEQ ID NO: 108)
D[LM]DXFXK,
(SEQ ID NO: 95)
D[LMY]SGI,
(SEQ ID NO: 192)
D[MR]NX[ST][LW],
(SEQ ID NO: 11)
D[PA]X[ML]X[STA]XK,
(SEQ ID NO: 55)
D[SKH]NGT,
(SEQ ID NO: 54)
D[STRING[TS],
(SEQ ID NO: 188)
DSNG[ST],
(SEQ ID NO: 110)
DX[AC][GM][AFS]K,
(SEQ ID NO: 111)
DXAGXK,
(SEQ ID NO: 193)
DXNX[ST][CY][TW],
(SEQ ID NO: 60)
DXNX[TS]XK[YF],
(SEQ ID NO: 29)
DXSGIXXX[DGESN],
(SEQ ID NO: 57)
DXXG[TS][FWY][MV],
(SEQ ID NO: 14)
EGXLXMXK,
(SEQ ID NO: 85)
ETXS[LM]G,
(SEQ ID NO: 104)
FP[AP][MT]XXK,
(SEQ ID NO: 47)
FX[RK][RK]DT,
(SEQ ID NO: 146)
FXE[HR]XK[PR],
(SEQ ID NO: 118)
FXQVCXK,
(SEQ ID NO: 184)
FXR[AK]DXXXP,
(SEQ ID NO: 15)
FXXELXX[WY]F,
(SEQ ID NO: 117)
FXXV[MT]RK,
(SEQ ID NO: 8)
FXXVC[ENA]K,
(SEQ ID NO: 9)
HGWTK,
(SEQ ID NO: 99)
HXQCPXXXK,
(SEQ ID NO: 195)
IPGS[FY],
(SEQ ID NO: 172)
IXXAVSX[AS],
(SEQ ID NO: 140)
K[DE]X[IV]PXXD,
(SEQ ID NO: 203)
K[DS]G[ILV]XW,
(SEQ ID NO: 33)
K[FY][LM]XFXQ,
(SEQ ID NO: 72)
K[WY]X[AP]FXQ,
(SEQ ID NO: 169)
KAXSXXXXXQ,
(SEQ ID NO: 173)
KAXSXXXY,
(SEQ ID NO: 93)
KEXLXXWF,
(SEQ ID NO: 141)
KMXPWXQ,
(SEQ ID NO: 86)
KTPP[ILM],
(SEQ ID NO: 34)
KWXXFQQ,
(SEQ ID NO: 150)
KXG[ACT]PXXXXP,
(SEQ ID NO: 155)
KXP[FW]CP,
(SEQ ID NO: 21)
KXQTX[ST]M,
(SEQ ID NO: 91)
KXSXIEX[ILM],
(SEQ ID NO: 71)
KXX[AG]XQQ[FY],
(SEQ ID NO: 114)
KXX[IV]Q[FY]XD,
(SEQ ID NO: 39)
KXXGPXXLY,
(SEQ ID NO: 139)
KXXIPYXD,
(SEQ ID NO: 106)
LLPXXX[IL]D,
(SEQ ID NO: 32)
LPFQQ,
(SEQ ID NO: 46)
LX[AP]R[RKA]D,
(SEQ ID NO: 101)
LX[IL]LXXLN,
(SEQ ID NO: 123)
M[ST][ACT]AK,
(SEQ ID NO: 124)
MT[AT][AS][AK],
(SEQ ID NO: 27)
P[EDT]WRXY,
(SEQ ID NO: 61)
PNG[TS]XXP,
(SEQ ID NO: 52)
PQXXXDK[TSR],
(SEQ ID NO: 194)
PX[ANP][QR]XDK,
(SEQ ID NO: 79)
PXWR[IV]Y,
(SEQ ID NO: 148)
PXWXXG[LM]D,
(SEQ ID NO: 162)
PXXRAXSXX[HPW],
(SEQ ID NO: 164)
PXXXRAXSXG,
(SEQ ID NO: 40)
QGPT[LI],
(SEQ ID NO: 98)
QGPTXP,
(SEQ ID NO: 22)
QX[AS]MXPK,
(SEQ ID NO: 137)
QX[NS]FKXX[FL],
(SEQ ID NO: 17)
QX[PT]C[LM]XK,
(SEQ ID NO: 18)
QXP[MQ]LXK,
(SEQ ID NO: 136)
QXSFKXX[FMW],
(SEQ ID NO: 19)
QXXC[LM]XKXXF,
(SEQ ID NO: 87)
R[EHS]XXEXXLF,
(SEQ ID NO: 145)
R[FH]PEK,
(SEQ ID NO: 198)
RD[ST]XXPXR,
(SEQ ID NO: 157)
RMADX[AHR],
(SEQ ID NO: 51)
RMADXXR,
(SEQ ID NO: 82)
RS[ILMV]AS,
(SEQ ID NO: 77)
RXPX[ST]L[DE],
(SEQ ID NO: 78)
RXPX[ST]LE,
(SEQ ID NO: 89)
RXX[CI]EXX[IL]F,
(SEQ ID NO: 5)
RXXQE[DN]XXF,
(SEQ ID NO: 88)
RXXQEXXLF,
(SEQ ID NO: 135)
SAXY[AMS]XK,
(SEQ ID NO: 130)
STXXL[ST]XK,
(SEQ ID NO: 90)
SXXEDXLF,
(SEQ ID NO: 129)
TPMXXX[ST]XK,
(SEQ ID NO: 158)
WAXMAD,
(SEQ ID NO: 144)
WF[DE]XID,
(SEQ ID NO: 120)
WWXXL[LMT]XK,
(SEQ ID NO: 183)
WX[KR][AKR]D[ST],
(SEQ ID NO: 42)
WX[RK]A[DV]S,
(SEQ ID NO: 50)
WX[RK]MAD,
(SEQ ID NO: 121)
WXX[IL]M[AL]K,
(SEQ ID NO: 181)
Y[KR]XD[ST][NY],
(SEQ ID NO: 154)
YCXAXKXC,
(SEQ ID NO: 151)
YWXYFXK,
(SEQ ID NO: 143)
YX[IR][FY]ML.

108-219. (canceled)
